(19) United States
(12) Patent Application Publication
(10) Pub. No.: US 2012/0253102 A1
(43) Pub. Date: Oct. 4, 2012

(54) EXTERNAL MAGNETIC FORCE FOR TARGETED CELL DELIVERY WITH ENHANCED CELL RETENTION

(75) Inventors: Eduardo Marban, Beverly Hills, CA (US); Ke Cheng, Los Angeles, CA (US)

(73) Assignee: CEDARS-SINAI MEDICAL CENTER, Los Angeles, CA (US)

(21) Appl. No.: 13/504,747

(22) PCT Filed: Oct. 27, 2010
(86) PCT No.: PCT/US10/54358

§ 371 Date: Apr. 27, 2012

Related U.S. Application Data
(60) Provisional application No. 61/255,438, filed on Oct. 27, 2009.

(51) Int. Cl. A61M 37/00; A61N 2/10; C12N 5/071

(52) U.S. Cl. 600/12; 435/325

(57) ABSTRACT

Disclosed herein are compositions and methods for the improved delivery of cells to a target tissue. In some embodiments, the compositions comprise stem cells, in particular cardiac stem cells, and the target tissue is damaged or diseased cardiac tissue. In several embodiments, the methods, in combination with the compositions, yield enhanced delivery, retention, and/or engraftment of the cells into the target tissue, thereby inducing improved functional recovery.

### CROSS-REFERENCE TO RELATED APPLICATIONS

This application claims the benefit of U.S. Provisional Application No. 61/255,438, filed on Oct. 27, 2009 the disclosure of which is expressly incorporated by reference herein.

### STATEMENT REGARDING FEDERALLY SPONSORED R&D

This invention was made with Government support under NIH Contract HL083109, an RO1 grant awarded by the National Heart, Lung, and Blood Institute. The Government has certain rights in the invention.

### BACKGROUND

1. Field of the Invention

Embodiments of the present application relate generally to compositions and methods for the enhanced delivery, retention and/or the engraftment of cells in a target tissue or organ using cell magnetization, optionally in combination with one or more vascular permeability agents. In particular, cardiac cells may be delivered to damaged cardiac tissue and the enhanced delivery, retention and/or the engraftment of the cells facilitates repair and/or regeneration of cardiac tissue.

2. Description of the Related Art

Heart disease is a leading cause of fatalities in modern societies. In recent years, the use of stem cells has offered tremendous potential for various cardiac diseases. However, a major challenge of delivering and localizing cells to a target tissue or organ such as the heart remains. Additionally, cell retention rate after delivery is low, regardless of the delivery method. For example, the low cell retention rate in the heart is known to be largely due to the wash-out effect caused by blood flow, coupled, in contracting organs like the heart in particular, with extrusion of injected cells at the injection site. It is also difficult to direct cells to a particular organ or tissue where their healing action is intended. Accordingly, there is a need in the art to provide methods to provide targeted cell delivery with enhanced cell delivery, engraftment, and/or retention.

### SUMMARY

In several embodiments, there is provided a method for the targeted delivery of agents to a damaged tissue, comprising delivering a magnetically labeled agent to one or more delivery sites of a damaged tissue and transiently applying a magnetic field around or adjacent to the damaged tissue, wherein the application of the magnetic field enhances the efficacy of the agent in repairing the damaged tissue. In some embodiments, the damaged tissue is a heart. In some embodiments, the magnetically labeled agent is delivered to the damaged heart while the damaged heart is beating. In some embodiments, the magnetically labeled agent is a cell. In some embodiments, the magnetically labeled agent is a stem cell. In some embodiments, the stem cell is a cardiac stem cell. In some embodiments, the cardiac stem cell is a cardiosphere-derived stem cell. In some embodiments, the magnetically labeled agent is a drug.

In several embodiments there is provided a method for magnetically targeting cells into a heart to repair damaged cardiac tissue, comprising delivering magnetically-labeled cardiac stem cells to one or more delivery sites of an actively contracting heart and transiently applying a magnetic field around or adjacent to the damaged cardiac tissue, wherein the magnetic field enhances the short-term retention and long-term engraftment of the delivered cells which facilitates the repair of the damaged cardiac tissue. In some embodiments, the cardiac tissue is damaged as a result of injury or disease, and has reduced cardiac function. In some embodiments, the active contraction of the heart induces an efflux of the delivered cells away from the delivery site. In several embodiments application of the magnetic field counteracts the efflux of the magnetically labeled cells and thereby enhances retention and engraftment of the delivered cells. In some embodiments, this provides long-term functional and anatomical improvements in the region of damaged cardiac tissue. In some embodiments, delivery of the magnetically-labeled stem cells does not preferentially attract macrophages to the region of damaged cardiac tissue. Thus, in some embodiments, the limited inflammatory response facilitates repair of the tissue.

In some embodiments, the cardiac stem cells comprise cardiosphere derived cells. In some embodiments, cardiospheres themselves are delivered. In some embodiments, the stem cells are adult stem cells. In some embodiments, other types of stem cells are used, such as bone marrow derived stem cells, mesenchymal stem cells, or embryonic stem cells. In some embodiments, stem cells are not used, but other types of cells are used, such as fibroblast, hepatocytes etc. In some embodiments, the delivered cells are autologous while in other embodiments allogeneic cells are delivered. In several embodiments the delivery route is intramyocardial. In several embodiments, the delivery route of the cells is intracoronary. Other delivery routes (e.g., intravenous, etc.) are used in other embodiments.

In several embodiments, the magnetic labels comprise super paramagnetic iron oxide (SPIO) particles. In some embodiments the SPIO particles comprise superparamagnetic microspheres (SPM). In some embodiments, other metal or magnetic-responsive materials are used as labels. In some embodiments, the labels are internalized into the cell, while in other embodiments, the labels are external. In some embodiments, combinations of internal and external labels are used in order to maximize the targeting potential of a cell. In some embodiments, biological targeting mechanism (e.g., antibodies) are used to supplement the targeting of the labeled cells and/or to enrich certain types of cells prior to delivery (or enrich the deposition of the labeled cells at a specific antigen-presenting target site). In one embodiment, the ratio of label to cell is about 500:1. In some embodiments, such as those with antigenic co-labeling, lower ratios may be used. In tissues that are deeper from the surface of a subject (e.g., the magnetic field may be weaker at a more internal target site), larger ratios are used. In some embodiments, the degree of magnetic label associated with the delivered cells (or other agent) is reduced over time.

In several embodiments, the magnetic field is applied via one or more magnetic sources positioned external to the heart. In several embodiments the magnetic field is applied via a catheter having a magnetic tip. In several embodiments, the magnetic-tipped catheter is introduced into a chamber of the heart and the agent is delivered to the endocardium. In some embodiments, the catheter additionally comprises a screw or barbed tip to assist in anchoring the catheter to the heart wall. In one embodiment, the screw tip prevents disengagement of the catheter from the endocardium due to the beating of the heart. In some embodiments, the generated magnetic field has a field strength of about 0.1 Tesla to about 100 Tesla. In one embodiment the magnetic field has a field strength of about 0.5 to about 1.3 Tesla. In several embodiments, the magnetic field is applied for a time period ranging from about 1 minute up to about 5 hours. In some embodiments, the magnetic field is applied for about 1 minute to 5 minutes, about 5 minutes to about 10 minutes, about 10 minutes to about 20, about 20 minutes to about 30 minutes, and overlapping ranges thereof. In several embodiments, the magnetic field is applied for about 5-15 minutes, including about 6, 7, 8, 9, 10, 11, 12, 13, or 14 minutes. In several embodiments, magnetic targeting is unexpectedly efficacious given that the magnetic particles in or on the cells will be subject to a particular magnetic force for a particular time, which places structural stresses on the cells that would not likely exist in an endogenous context. These stresses may negatively impact the viability and/or functionality of the cells such that retention, engraftment and overall functional improvement post-delivery would be compromised. In contrast, such stresses would be less likely to negatively affect a magnetically targeted drug (e.g., a chemical compound) for example. Advantageously, however, the methods and compositions provided herein, in several embodiments, still yield enhanced repair and or regeneration of cardiac tissue despite the non-endogenous forces exerted on the delivered cells.

In several embodiments, short term retention is enhanced by at least 10% as compared to non-magnetically targeted cells. In some embodiments, short term retention is increased by 15, 20, 25, 30, 40% or more. In some embodiments, long term engraftment is enhanced by at least 10% as compared to non-magnetically targeted cells. In some embodiments, long term engraftment is increased by 15, 20, 25, 30, 40% or more. In some embodiments, the delivered cells engraft into the damaged cardiac tissue as focal patches of cells.

In several embodiments, the cardiac tissue has suffered damage due to an acute injury to the heart. In some embodiments, the acute injury comprises a myocardial infarction. In some embodiments, however, the damage to the cardiac tissue results from chronic stress or disease of the heart. In some embodiments, one or more of the following is responsible for the damage: chronic heart failure, systemic hypertension, pulmonary hypertension, valve dysfunction, congestive heart failure, and coronary artery disease. In some embodiments, the chronic disease precipitates an acute injury.

In some embodiments, the damaged cardiac tissue is one of the epicardium, endocardium, and myocardium. In some embodiments, however, more than one of these cardiac tissues is simultaneously damaged and subsequently repaired.

In some embodiments, the methods provided herein lead to a functional improvement in the damaged heart which is manifest as increased cardiac output. In some embodiments, the increase in cardiac output comprises an increase in left ventricular ejection fraction. In some embodiments, delivery of the magnetized cells yields an increase of at least 5% in the left ventricular ejection fraction. In one embodiment left ventricular ejection fraction is increased by about 10%. In some embodiments, the amount of viable cardiac tissue is increased in addition to the increased cardiac output. In some embodiments, anatomical improvements in the damaged heart tissue, such as an increase in cardiac wall thickness, are detected. In some embodiments, wherein the damaged cardiac tissue is a result of a myocardial infarction the methods provided herein result in decreases in scar tissue formation. In some embodiments, the functional and/or anatomical improvements in the damaged cardiac tissue are due to proliferation of the delivered cells within or adjacent to the region of damaged cardiac tissue. In some embodiments, the functional and/or anatomical improvements in the damaged cardiac tissue is due to paracrine modulators released from the delivered cells, wherein the paracrine modulators improve the viability of cardiac tissue and/or recruit endogenous cardiac cells to the region of damaged cardiac tissue.

In several embodiments, there is provided a method for the repair or regeneration of damaged cardiac tissue, comprising delivering magnetically-labeled stem cells to a subject having a heart comprising a region of damaged cardiac tissue with compromised cardiac function, wherein the stem cells are cardiac stem cells, applying a magnetic field around or adjacent to the damaged cardiac tissue leading to enhanced delivery, retention, or engraftment of the magnetically-labeled stem cells which results in functional improvement in the region of damaged cardiac tissue. In some embodiments, the magnetically-labeled stem cells are delivered within or adjacent to the region of damaged cardiac tissue. In other embodiments, the cells are delivered systemically and targeted to the heart. In some embodiments, the magnetic field enhances short term retention and long-term engraftment of the magnetically-labeled stem cells. In some embodiments, the functional improvement comprises an increase in left ventricular ejection fraction. In some embodiments, in addition to the increases in LVEF, an increase in viable cardiac tissue is also recognized. In some embodiments, the methods provided herein also result in an increase in cardiac wall thickness.

In several embodiments, there is provided a method for improving the function of cardiac tissue damaged as a result of a myocardial infarction, comprising delivering, via a catheter, magnetically-labeled cardiac stem cells to a subject having been afflicted with a myocardial infarction and generating a magnetic field from the catheter, wherein the magnetic field enhances the retention of the magnetically-labeled cardiac stem cells at the region of damaged cardiac tissue. In some embodiments, the enhanced retention results in enhanced engraftment of the magnetically-labeled cardiac stem cells. In some embodiments, the enhanced retention and engraftment produce healthy myocardium at the region of damaged cardiac tissue, and the healthy myocardium results in functional improvement of the damaged cardiac tissue.

In several embodiments, there is provided the use of magnetically-labeled cardiac stem cells for the repair of damaged cardiac tissue. In some embodiments, magnetically-labeled cardiac stem cells are cardiosphere-derived cells labeled with SPM particles. In some embodiments, the magnetically-labeled cardiac stem cells are suitable for delivery to the heart of a subject having damaged cardiac tissue. In some embodiments, application of a magnetic field applied around the damaged cardiac tissue results in increased retention of delivered magnetically-labeled cardiac stem cells and the increased retention leads to functional improvement of the damaged cardiac tissue, thereby repairing the damaged cardiac tissue.

In several embodiments, the methods provided herein relate generally to compositions and methods for the delivery, retention and/or the engraftment of cells in a target tissue or organ, e.g., the heart, using cell magnetization, optionally in combination with one or more vascular permeability agents. In some embodiments, the methods and compositions provided herein comprise stem cells, including cardiac stem cells, labeled with magnetic particles for delivery to a target tissue or organ, e.g., the heart.

In one embodiment, provided herein is a method for delivering cardiosphere-derived cells (CDCs) to a cardiac tissue, comprising: (a) labeling the CDCs with magnetic particles; (b) contacting the CDCs with the cardiac tissue; and (c) applying a magnetic field around or adjacent to the cardiac tissue, In some embodiments, the magnetic field is an external magnet.

In another embodiment, there is provided a method for retaining CDCs in a cardiac tissue, comprising: (a) labeling the CDCs with magnetic particles; (b) contacting the CDCs with the cardiac tissue; and (c) applying a magnetic field around or adjacent to the cardiac tissue, such that the CDCs are retained in the cardiac tissue. In some embodiments, the magnetic field is an external magnet.

In another embodiment, there is provided a method for engraftment of CDCs in a cardiac tissue, comprising: (a) labeling the CDCs with magnetic particles; (b) contacting the CDCs with the cardiac tissue; and (c) applying a magnetic field around or adjacent to the cardiac tissue, such that engraftment of the CDCs occurs. In some embodiments, the magnetic field is an external magnet. In some embodiments, the engrafted CDCs generate additional cardiac cells.

In another embodiment, there is provided a method for treating an injured cardiac tissue in a subject, comprising: (a) labeling CDCs with magnetic particles; (b) contacting the CDCs with the injured cardiac tissue; and (c) applying a magnetic field around or adjacent to the injured cardiac tissue, such that retention and/or targeting are enhanced and wherein the cardiac tissue is treated. In some embodiments, the magnetic field is an external magnet.

In another embodiment, there is provided a method for delivering cells to a tissue or organ, comprising: (a) labeling the cells with magnetic particles; (b) contacting the cells and a vascular permeability agent with the tissue or organ; and (c) applying a magnetic field around or adjacent to the tissue or organ. In some embodiments, the magnetic field is an external magnet.

In another embodiment, there is provided a method for retaining cells in a tissue or organ, comprising: (a) labeling the cells with magnetic particles; (b) contacting the cells and a vascular permeability agent with the tissue or organ; and (c) applying a magnetic field around or adjacent to the tissue or organ. In some embodiments, the magnetic field is an external magnet.

In another embodiment, there is provided a method for engraftment of cells in a tissue or organ, comprising: (a) labeling the cells with magnetic particles; (b) contacting the cells and a vascular permeability agent with the tissue or organ; and (c) applying a magnetic field around or adjacent to the tissue or organ, such that engraftment of the cells occurs. In some embodiments, the magnetic field is an external magnet.

In another embodiment, there is provided a method for treating an injured tissue or organ in a subject, comprising: (a) labeling cells with magnetic particles; (b) contacting or perfusing the injured tissue or organ with the cells; and (c) applying a magnetic field around or adjacent to the injured tissue or organ, such that the tissue or organ is treated. In some embodiments, the magnetic field is an external magnet.

In another embodiment, there is provided a method of treating or otherwise managing a cancer or tumor, comprising: (a) labeling anti-tumor cells with magnetic particles; (b) contacting the cells with the cancer or tumor; and (c) applying a magnetic field around or adjacent to the cancer or tumor. In some embodiments, magnetic field is an external magnet.

In additional embodiments, there are provided compositions comprising CDCs comprising magnetic particles. In some embodiments, the compositions further comprise a vascular permeability agent. Kits comprising said compositions in one or more containers and optionally instructions for use are also provided herein.

In additional embodiments, there are provided compositions comprising a vascular permeability agent and cells comprising magnetic particles. Kits comprising said compositions in one or more containers and optionally instructions for use are also provided herein.

### Terminology

Unless defined otherwise, all technical and scientific terms used herein have the same meaning as is commonly understood by one of ordinary skill in the art. All patents, applications, published applications and other publications are incorporated herein by reference in their entirety. In the event that there is a plurality of definitions for a term herein, those in this section prevail unless stated otherwise.

The term “about” or “approximately” shall be given its ordinary meaning and shall also refer to ranges within 20% or less in some contexts, within 10% or less in some contexts, or within 5% or less (or 1% or less), in some contexts, of a given value or range.

As used herein, “administer,” “administration” and “administering” shall be given their ordinary meaning and shall also refer to the act of injecting, applying, or otherwise physically delivering a substance as it exists outside the body (e.g., labeled cells, vascular permeability agents and/or therapeutic agents provided herein) into a patient, such as by, but not limited to, intramyocardial, pulmonary (e.g., inhalation), mucosal (e.g., intranasal), intradermal, intravenous, surgical, intramuscular delivery and/or any other method of physical delivery described herein or known in the art. When a disease, or a symptom thereof, is being treated, administration of the substance typically occurs after the onset of the disease or symptoms thereof. When a disease, or symptom thereof, is being prevented, administration of the substance typically occurs before the onset of the disease or symptoms thereof. Such administration, in some embodiments, results in the delivered substance (e.g., labeled CDCs) contacting the target tissue or organ (e.g., cardiac tissue).

As used herein, the term “allogeneic” shall be given its ordinary meaning and shall also refer to organs, tissues, cells, fluids or other bioactive molecules that are from the same species but antigenically or genetically distinct.

The term “angiogenic factor” or “angiogenic agent” as used herein shall be given their ordinary meaning and shall also refer to a molecule capable of activating or otherwise promoting angiogenesis, which is a process by which new blood vessels grow and develop.

The term “autologous” as used herein shall be given its ordinary meaning and shall also refer to organs, tissues, cells, fluids or other bioactive molecules that are reimplanted in the same individual that they originated from. Non-limiting examples of autologous transplants or grafts include bone, bone marrow, skin biopsy, heart biopsy, cartilage and blood and stem cells, e.g., CDCs.

The term “cardiac cells” as used herein shall be given its ordinary meaning and shall also refer to any cells present in the heart that provide a cardiac function, such as heart contraction or blood supply, or otherwise serve to maintain the structure of the heart. Cardiac cells as used herein encompass cells that exist in the epicardium, myocardium or endocardium of the heart. Cardiac cells also include, for example, cardiac muscle cells or cardiomyocytes, and cells of the cardiac vasculatures, such as cells of a coronary artery or vein. Other non-limiting examples of cardiac cells include epithelial cells, endothelial cells, fibroblasts, cardiac conducting cells and cardiac pacemaking cells that constitute the cardiac muscle, blood vessels and cardiac cell supporting structure.

The term “cardiac function” shall be given its ordinary meaning and shall also refer to the function of the heart, including global and regional functions of the heart. The term “global” cardiac function as used herein shall be given its ordinary meaning and shall also refer to function of the heart as a whole. Such function can be measured by, for example, stroke volume, ejection fraction, cardiac output, cardiac contractility, etc. The term “regional cardiac function” shall be given its ordinary meaning and shall also refer to the function of a portion or region of the heart. Such regional function can be measured, for example, by wall thickening, wall motion, myocardial mass, segmental shortening, ventricular remodeling, new muscle formation, the percentage of cardiac cell proliferation and programmed cell death, angiogenesis and the size of fibrous and infarct tissue. In some embodiments, cardiac cell proliferation is assessed by the increase in the nuclei or DNA synthesis of cardiac cells, cell cycle activities or cytokinesis. In some embodiments, programmed cell death is measured by TUNEL assay that detects DNA fragmentation. In some embodiments, angiogenesis is detected by the increase in arteriolar and/or capillary densities. Techniques for assessing global and regional cardiac function are known in the art. For example, techniques that can be used to measure regional and global cardiac function include, but are not limited to, echocardiography (e.g., transthoracic echo cardiogram, transesophageal echocardiogram or 3D echocardiography), cardiac angiography and hemodynamics, radionuclide imaging, magnetic resonance imaging (MRI), sonomicrometry and histological techniques.

The term “cardiac tissue” as used herein shall be given its ordinary meaning and shall also refer to tissue of the heart, for example, the epicardium, myocardium or endocardium, or portion thereof, of the heart. The term “injured” cardiac tissue as used herein shall be given its ordinary meaning and shall also refer to a cardiac tissue that is, for example, ischemic, infarcted, reperfused, or otherwise focally or diffusely injured or diseased. Injuries associated with a cardiac tissue include any areas of abnormal tissue in the heart, including any areas caused by a disease, disorder or injury and includes damage to the epicardium, endocardium and/or myocardium. Non-limiting examples of causes of cardiac tissue injuries include acute or chronic stress (e.g., systemic hypertension, pulmonary hypertension or valve dysfunction), atheromatous disorders of blood vessels (e.g., coronary artery disease), ischemia, infarction, inflammatory disease and cardiomyopathies or myocarditis.

The term “effective amount” as used herein shall be given its ordinary meaning and shall also refer to the amount of a therapy (e.g., labeled cells, either alone or in combination with a vascular permeability agent and/or a therapeutic agent) which is sufficient to reduce and/or ameliorate the severity and/or duration of a given disease and/or a symptom related thereto. For example, in some embodiments of the compositions provided herein, the composition comprises an effective amount of cells, such as stem cells (e.g., CDCs), either alone or in combination with a vascular permeability agent and/or a therapeutic agent. In other embodiments, the methods provided herein comprise contacting or otherwise administering cells, such as stem cells (e.g., CDCs), either alone or in combination with a vascular permeability agent and/or a therapeutic agent.

The term “engraftment” as used herein shall be given its ordinary meaning and shall also refer to a process by which transplanted cells, for example, stem cells (e.g., autologous or allogeneic stem cells), are accepted by a host tissue, survive and persist in that environment, e.g., for a period of 24 hours or more. In some embodiments, the transplanted cells further reproduce.

In several embodiments, an “external magnet” shall be given its ordinary meaning and shall also refer to a magnet or a magnetic field placed outside of the body. In other embodiments, an “external magnet” refers to a magnet or a magnetic field placed in or adjacent to a tissue or organ (e.g., esophagus or colon) that can be reached, e.g., with a fibroscope or other similar devices. In some embodiments, an “external magnet” refers to a magnetic tip of a catheter that may, for example, be advanced into the heart or other location within the body.

The term “fragment,” “functional fragment” or similar term shall be given their ordinary meanings and shall also refer to a portion of an amino acid sequence (or polynucleotide encoding that sequence) that has at least about 70% of the function of the corresponding full-length amino acid sequence (or polynucleotide encoding that sequence). In some cases, functional fragment refers to an amino acid sequence or polynucleotide sequence that has at least about 80% or at least about 95% of the function of the corresponding full-length amino acid (or polynucleotide) sequence.

The terms “generate,” “generation” and “generating” as used herein shall be given their ordinary meanings and shall also refer to the production of new cardiac cells in a subject and optionally the further differentiation into mature, functioning cardiac cells. In some embodiments, generation of cardiac cells comprises regeneration of the cardiac cells. In some embodiments, generation of cardiac cells comprises improving survival, engraftment and/or proliferation of the cardiac cells.

The term “in combination” as used herein in the context of the administration of other therapies shall be given its ordinary meaning and shall also refer to the use of more than one therapy. The use of the term “in combination” does not restrict the order in which therapies are administered to a subject. A first therapy can be administered before (e.g., 1 minute, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject which had, has, or is susceptible to a given disease. Any additional therapy can be administered in any order with the other additional therapies. In some embodiments, the magnetically labeled cells provided herein can be administered in combination with one or more therapies (e.g., therapies that are not the magnetically labeled cells that are currently administered to prevent, treat, manage, and/or ameliorate a given disease or other symptom related thereto). Non-limiting examples of therapies that can be administered in combination with labeled cells provided herein include analgesic agents, anesthetic agents, antibiotics, or immunomodulatory agents or any other agent listed in the U.S. Pharmacopoeia and/or Physician's Desk Reference.

As used herein, the term “labeled cells,” “magnetic particle-labeled cells” “magnetically labeled cells” and “magnetized cells” are used interchangeably, shall be given their ordinary meanings, and shall also refer to cells, e.g., stem or progenitor cells, that have been rendered magnetic by, for example, incorporation of magnetic particles (e.g., ferromagnetic, paramagnetic, or superparamagnetic particles) into the cells (e.g., through cellular uptake of the particles). In several embodiments, cells are labeled through cell surface interactions with magnetic particles, e.g., an antigen coated magnetic particle that interacts with a cell surface receptor reactive to the antigen.

The term “magnetic particle” shall be given its ordinary meaning and shall also refer to any particle dispersible or suspendable in aqueous media without significant gravitational settling and separable from suspension by application of a magnetic field. Non-limiting examples of magnetic particles include microspheres, conjugates, micelles, colloids, liposomes, aggregates or complexes of a ferromagnetic, paramagnetic or superparamagnetic material.

The terms “manage,” “managing,” and “management” as used herein shall be given their ordinary meanings and shall also refer to the beneficial effects that a subject derives from a therapy (e.g., labeled cells provided herein) after the onset of a disease, which does not result in a cure of a disease. In some embodiments, a subject is administered one or more therapies to “manage” a given disease or one or more symptoms related thereto, so as to prevent the progression or worsening of the disease.

As used herein, the term “peri-infarct zone” shall be given its ordinary meaning and shall also refer to area at the junction between the normal tissue and the infarcted tissue, i.e., an area of a dying or dead heart tissue resulting from obstruction of blood flow to the heart muscle that results from a relative or absolute insufficiency of blood supply. In some embodiments of the methods provided herein, the magnetized cells are administered into the peri-infarct zone of the cardiac tissue.

As used herein, the terms “preserve,” “preservation of’ and “preserving” in the context of injured tissue shall be given their ordinary meanings and shall also refer to protection and/or maintenance of the cardiac tissue, or the functions thereof, such that the tissue is not further injured or compromised, or that the rate of further injury or compromise is slowed relative to the rate in the absence of the intervention at issue. In some embodiments, preserving injured cardiac tissue comprises prevention or reduction of apoptosis of cells (e.g., cardiomyocytes or stem cells). In some embodiments, preserving injured cardiac tissue comprises prevention or reduction of cell inflammation.

The terms “regenerate,” “regeneration” and “regenerating” as used herein in the context of injured tissue shall be given their ordinary meanings and shall also refer to the process of growing and/or developing new cardiac tissue in a heart or cardiac tissue that has been injured, for example, injured due to ischemia, infarction, reperfusion, or other disease. In some embodiments, cardiac tissue regeneration comprises activation and/or enhancement of cell proliferation. In some embodiments, cardiac tissue regeneration comprises activation and/or enhancement of cell migration.

The term “retention” as used herein shall be given its ordinary meaning and shall also refer to a process by which transplanted cells, for example, stem cells (e.g., autologous or allogeneic stem cells), are retained by a host tissue or organ, e.g., are accepted, survive and persist in that environment, e.g., for a period of minutes to hours. In some embodiments, the transplanted cells, e.g., stem cells, further reproduce. In some embodiments, increased retention promotes increased engraftment, which in turn promotes improved function of a targeted tissue or organ.

The term “stem cells” shall be given its ordinary meaning and shall also refer to cells that have the capacity to self-renew and to generate differentiated progeny. The term “pluripotent stem cells” shall be given its ordinary meaning and shall also refer to stem cells that can give rise to cells of all three germ layers (endoderm, mesoderm and ectoderm), but do not have the capacity to give rise to a complete organism.

The term “induced pluripotent stem cells” shall be given its ordinary meaning and shall also refer to differentiated mammalian somatic cells (e.g., adult somatic cells, such as skin) that have been reprogrammed to exhibit at least one characteristic of pluripotency (see, e.g., co-owned U.S. Application No. 61/116,623, filed Nov. 20, 2008), which is incorporated by reference herein.

The term “multipotent stem cells” shall be given its ordinary meaning and shall also refer to a stem cell that has the capacity to grow into a subset of the fetal or adult mammalian body's approximately 260 cell types. For example, some multipotent stem cells can differentiate into at least one cell type of ectoderm, mesoderm and endoderm germ layers. A “progenitor” cell shall be given its ordinary meaning and shall also refer to a cell that has the ability to self-renew, generally for a limited number of times, and can also give rise to a particular cell type or limited group of cell types. The term “embryonic stem cells” shall be given its ordinary meaning and shall also refer to stem cells derived from the inner cell mass of an early stage embryo, e.g., human, that can proliferate in vitro in an undifferentiated state and are pluripotent.

The term “cardiac stem cells” shall be given its ordinary meaning and shall also refer to stem cells obtained from or derived from a cardiac tissue. The term “cardiosphere-derived cells (CDCs)” as used herein shall be given its ordinary meaning and shall also refer to undifferentiated cells that grow as self-adherent clusters from subcultures of postnatal cardiac surgical biopsy specimens. CDCs can express stem cell as well as endothelial progenitor cell markers, and typically possess properties of adult cardiac stem cells. See, for example, Davis et al. (2009) PLoS One 4(9):e7195, which is incorporated herein by reference in its entirety. For example, human CDCs can be distinguished from human cardiac stem cells in that human CDCs typically do not express multidrug resistance protein 1 (MDR1; also known as ABCB1), CD45 and CD133 (also known as PROM1). See, e.g., Passier et al. (2008) Nature 453:322, which is incorporated herein by reference in its entirety. CDCs are capable of long-term self-renewal, and can differentiate in vitro to yield cardiomyocytes or vascular cells after ectopic (dorsal subcutaneous connective tissue) or orthotopic (myocardial infarction) transplantation in SCID beige mouse. See also U.S. Pub. No. 2008/0267921, which is incorporated herein by reference in its entirety.

The term “bone marrow stem cells” shall be given its ordinary meaning and shall also refer to stem cells obtained from or derived from bone marrow. The term “placenta-derived stem cells” or “placental stem cells” shall be given their ordinary meanings and shall also refer to stem cells obtained from or derived from a mammalian placenta, or a portion thereof (e.g., amnion or chorion). See, for example, U.S. Pat. No. 7,468,276 and US Patent Publication No. US 2007/0275362, herein incorporated by reference. The term “amniotic stem cells” shall be given its ordinary meaning and shall also refer to stem cells collected from amniotic fluid or amniotic membrane. The term “embryonic germ cells” shall be given its ordinary meaning and shall also refer to cells derived from primordial germ cells, which exhibit an embryonic pluripotent cell phenotype. The term “spermatocytes” shall be given its ordinary meaning and shall also refer to male gametocytes derived from a spermatogonium.

As used herein, the terms “subject” and “patient” are used interchangeably and shall be given their ordinary meaning. As used herein, a subject can be a mammal such as a non-primate (e.g., cows, pigs, horses, cats, dogs, rats, rabbits, etc.) or a primate (e.g., monkey and human), for example, having an injured tissue or organ (e.g., cardiac tissue). In some embodiments, the subject is a human. In one embodiment, the subject is a mammal with acute heart failure. In another embodiment, the subject is a mammal with chronic heart failure.

The term “synergistic” as used herein shall be given its ordinary meaning and shall also refer to a combination of for example, stem cells, and one or more therapeutic agents or vascular permeability agents, which is more effective than the additive effects of any two or more single agents (e.g., stem cells and one therapeutic agent; or two therapeutic agents without stem cells).

The term “therapeutic agent” or “therapeutic drug” as used herein shall be given its ordinary meaning and shall also refer to any therapeutically active substance that is delivered to a bodily conduit of a living being to produce a desired, usually beneficial, effect.

As used herein, the term “therapy” shall be given its ordinary meaning and shall also refer to any protocol, method and/or agent that can be used in the management, treatment and/or amelioration of a given disease, or a symptom related thereto. In some embodiments, the terms “therapies” and “therapy” refer to a biological therapy, supportive therapy, and/or other therapies known to one of skill in the art, such as medical personnel, useful in the management or treatment of a given disease, or symptom related thereto.

As used herein, the terms “treat,” “treatment” and “treating” shall be given their ordinary meanings and shall also refer to the reduction or amelioration of the progression, severity, and/or duration of a tissue injury or a symptom thereof. For example, with respect to cardiac tissue injury, treatment as used herein includes, but is not limited to, preserving the injured cardiac tissue, regenerating new cardiac tissue, increasing blood flow to the injured tissue, increasing myocardial perfusion, improving global cardiac function (e.g., stroke volume, ejection fraction, and cardiac output) and regional cardiac function (e.g., ventricular wall thickening, segmental shortening and heart pumping).

The terms “vascular permeability” or “microvascular permeability” shall be given their ordinary meaning and shall also refer to the capacity of a blood vessel wall to allow for the movement of small molecules (e.g., ions, water, nutrients) or cells in and out of the vessel.

The terms “vascular permeability agent” or “microvascular permeability agent” shall be given their ordinary meanings and shall also refer to an agent which increases the capacity of a blood vessel wall to allow for the movement of small molecules (e.g., ions, water, nutrients) or cells in and out of the vessel.

### BRIEF DESCRIPTION OF THE DRAWINGS

FIG. 1 depicts the quantification of cellular superparamagnetic iron oxide (SPIO) uptake. Rat CDCs were labeled with SPIO (0.9-μm diameter, Bangs Laboratories, IN) with a dragon green fluorescence tag overnight prior to being examined under a fluorescence microscopy (Excitation 488 nm; Emission 520 nm). A green color indicates that the cells were successfully labeled with the SPIO.

FIGS. 2A-2D depict the flow cytometry analysis of SPIO labeling efficiency. Rat CDCs were labeled with SPIO (0.9-μm diameter, Bangs Laboratories, IN) with a dragon green fluorescence tag overnight. Compared to the control group (upper panels A and B), the histogram shifts to the right hand side as SPIO-labeled cells exhibit a green fluorescence (lower panels C and D).

FIGS. 3A-3B depict the cell retention assessed by white light imaging. SPIO labeled-CDCs derived from syngeneic male Wistar Kyoto (WK) rats were injected intramyocardially into the ischemic region of female rats. White light imaging revealed that SPIO-labeled cells, which have a dark brown color, were attracted towards the magnet and “trapped” around the infarct, while the majority of non-targeted cells were washed out immediately after injection. After 24 hours, examination of excised hearts indicated the animal exposed to the magnet has more cells in the heart (panel A) compared to the control group (panel B).

FIG. 4 depicts the cell retention assessed by quantitative PCR. The data revealed that magnetically targeted CDCs exhibited approximately 3-fold enhanced of cell retention compared to non-targeted cells at 24 hour after injection (20.7±4.3% vs. 7.6±1.2%, n=7, p<0.0005).

FIGS. 5A-5D depict acute cell retention assessed by fluorescence imaging. At 24 hour after cell transplantation, fluorescence imaging (FLI) images revealed that the magnetic targeting group had more cell retention in the heart (compare panel B, magnetic targeting, to panel A, no magnetic targeting, but less off-target expression in other organs such as lung and spleen (panel D), compared to the non targeting group (panel C).

FIGS. 6A-6H depict long-term (3 week) cell retention assessed by fluorescence imaging. At 3 weeks after transplantation, FLI revealed that targeted CDCs exhibited approximately 4-fold enhanced of retention (panels A-C) compared to non-targeted cells (panels D-F). No cells were found in the lungs after 3 weeks (panel G). Panel H depicts a histogram reflecting the difference in cell retention between magnetically targeted and non targeted cells.

FIG. 7 depicts the cardiac function assessed by the left ventricular ejection fraction (LVEF). Cardiac function were measured on the day of cell transplantation (as the baseline) and 3 weeks after cell transplantation (as the end point). Ventricular performance was quantified by echocardiography (RMV-707B scan head, Vevo770, Visual Sonics). In this figure, “NS” refers to “not significant” and “LVEF refers to “left ventricle ejection fraction.” Values are reported as mean±SEM. Sham is performed by PBS injection. The data indicate that the heart function of SPIO-labeled and magnetically targeted CDCs significantly outperformed the two control groups: 1) SPIO-labeled CDCs without targeting; and 2) unlabeled CDCs.

FIG. 8 depicts the left ventricle ejection fraction change (ALVEF) from the baseline at 3 weeks after cell transplantation. Cardiac function was measured on the day of cell transplantation (as the baseline) and 3 weeks after cell transplantation (as the end point). Ventricular performance was quantified by echocardiography (RMV-707B scan head, Vevo770, Visual Sonics). In this figure, “NS” refers to “not significant” and “LVEF refers to “left ventricle ejection fraction.” Values are reported as mean±SEM. Sham is performed by PBS injection. The data indicated that the heart function of SPIO-labeled and magnetically targeted CDCs significantly outperformed the two control groups: 1) SPIO-labeled CDCs without targeting; and 2) regular CDCs without labeling.

FIG. 9 depicts a study scheme of assessing cardiac retention of iron-loaded cells in a porcine model. Group 1 consists of donor animals of CDCs. Groups 2-5 consist of recipient animals of CDCs, either with (Groups 4 and 5) or without (Groups 2 and 3) acute myocardial infarction.

FIG. 10 depicts a randomized functional study of intracoronary infusion of iron-loaded cells in a porcine model. Animals in Group 6 (the control group) receive intracoronary infusion of saline with magnetic attraction. Animals in Groups 7 and 8 receive intracoronary infusion of iron-loaded cells, with (Group 7) or without (Group 8) magnetic attraction.

FIGS. 11A-11D depict analysis of SPM labeling of CDCs. Panel A depicts rat CDCs that were SPM labeled as compared to non-labeled cells (inset) and histochemically stained. Panel B depicts rat CDCs that were labeled with fluorescently conjugated SPMs as compared to non-labeled cells (inset). Panels C and D depict a representative flow cytometry histogram and dot plot, respectively, of SPM-labeled and unlabeled CDCs.

FIGS. 12A-12J depict various data collected related to SPM labeling and cell death or function. Panels A-C depict TUNEL staining results after CDCs were labeled with various ratios of SPM:cells (500:1 in Panel A; 2000:1 in Panel B; and 4000:1 in Panel C). Flow cytometry analysis of Annexin staining from non-labeled (Panel D) and SPM-labeled CDCs (Panel D) as well as quantification of apoptotic versus necrotic cells (Panel F) are also shown. Panel G depicts cell viability as assessed by Trypan Blue exclusion. Panel H depicts analysis of the proliferation of labeled CDCs versus non-labeled CDCs. Panel I depicts the adhesion potency of labeled versus non-labeled CDCs and Panel J depicts expression of various phenotypic markers in response to cell labeling.

FIGS. 13A-13O depict additional analysis of SPM labeling on cell death. Varied SPM:cell ratio were used (500:1 in Panels A,D, G, and J; 2000:1 in Panels B, E, H and K; and 4000:1 in Panels C, F, I and L). Apoptotic cells are indicated by white arrowheads. Control (non-labeled) CDCs are shown in Panels M, N, and O.

FIGS. 14A-14M depict the effects of SPM labeling on reactive oxygen species (ROS) formation in CDCs. SPM:cell ratios of 500:1 were used in Panels A-D. Panels E-H show unlabeled CDCs. Panels I-L show CDCs exposed to hydrogen peroxide for 24 hours. Panel M depicts a histogram of the generation of ROS under various labeling conditions.

FIGS. 15A-15E depict a procedure for the attraction of labeled CDCs through the use of a magnet.

FIGS. 16A-16K depict increases in short-term cell retention in target tissues and reductions in off-target migration of cells through magnetic targeting. Panels A and B are representative hearts from animals treated with labeled CDCs (Panel A) and labeled CDCs targeting with a magnet (Panel B). Panels C-K are representative images of organs harvested 24 hours after cell injection. Greater fluorescence (greater labeled cell retention) was detected in hearts from the magnet group (compare Panel F to Panel C). Less fluorescence was detected in lung and spleen of the magnet treated group (compare (Panels G and H to Panels D and E). Negative controls (non-labeled cells) are shown in Panels I-K.

FIGS. 17A-17E depict the effects of magnetic targeting on short-term cell retention and long-term engraftment. Panel A depicts percentage of cell retention 24 hours after injection and Panel B depicts percentage of cell retention 3 weeks after injection. Panels C and D are representative fluorescent images from non-targeted, labeled CDCs and magnetically targeted labeled CDCs, respectively. Panel E depicts quantification of fluorescence from the two treatment groups.

FIG. 18A-18H depict morphometric heart analysis of various treatments. Panels A-D are representative myocardial sections stained with Mason's trichrome in order to evaluate scar tissue deposition 3 weeks after the indicated treatment. Panels E-H are histograms depicting the viable cardiac tissue, scar tissue, infracted wall thickness, and left ventricular expansion index, respectively, for the various treatment groups.

FIGS. 19A-19D depict changes in various functional parameters in response to magnetic targeting of cells. Panel A shows baseline versus 3-week post treatment left ventricular ejection fraction for hearts treated with magnetically targeted CDCs, labeled but non-targeted CDCs, CDCs alone, and control hearts. Panel B depicts the change from baseline for each group. Panel C depicts a linear regression of left ventricular ejection fraction at 3 weeks versus cell retention at 3 weeks. Panel D depicts a linear regression of left ventricular ejection fraction at 3 weeks versus viable myocardium at 3 weeks.

FIGS. 20A-20F depicts experimental results that indicate that injection of SPMs alone is not responsible for the improvements in heart function detected with SPM-labeled CDCs. Panel A shows that injection of SPM alone yields a reduction in LVEF after 3 weeks, a pattern similar to that of a PBS injection (Panel B). Panels C—F are fluorescent immunohistochemistry images of SPM-injected heart at 3 weeks. SPM were present in the myocardium (Panel E), with many taken up by macrophages (Panel F).

FIGS. 21A-21F depicts analysis of cell engraftment and inflammatory responses in response to various treatments. Panels A-D are confocal images depicting the co-localization of macrophages in the cardiac tissue and SPM-labeled and magnetic targeted CDCs (Panel A), SPM-labeled and non-targeted CDCs (Panel B), CDCs (Panel C), and control injections (Panel D). Panel E shows a histogram depicting the number of GFP-positive and CD-68 positive (macrophages) from the various treatments. Panel F shows the number of GFP positive cells versus the number of events recorded.

FIGS. 22A-22F depict colocalization of SPM, GFP (CDCs) and CD-68 (macrophages). Panel A indicates that, at 24 hours post-injection, the majority of CDCs were SPM positive. Panel B shows that minimal numbers of SPM cells were colocalized with macrophages. At 3 weeks, few CDCs still contained SPMs (Panel C), while the majority of macrophages were SPM positive (Panel D). Panels E and F depict quantification of SPM/GFP and SPM/CD-68 colocalization at 24 hours and 3 weeks post-injection.

FIGS. 23A-23E depicts data relating to cardiac differentiation of transplanted CDCs. Panel A depicts shows SPM-labeled CDCs targeted with a magnet that co-localize with alpha-sarcomeric actin, indicating that these CDCs participated in regeneration of myocardium. Panel B depicts the quantification of GFP positive/alpha sarcomeric actin positive cells. Panel C depicts the quantification of GFP negative/alpha sarcomeric actin positive cells. Panel D depicts the percentage distribution of recipient and donor myocytes (both mature and immature) in the increment from the SPM-labeled CDC group to the SPM-labeled and magnetically targeted group. M indicates mature donor-derived cardiomyocytes, IM indicates immature donor-derived cardiomyocytes, and R indicates recipient-derived cardiomyocytes. Panel E depicts a potential mechanism of magnetic targeting therapy.

FIGS. 24A-24F depicts the expression of cardiac markers in SPM positive/GFP positive cells in an SPM-labeled CDC magnetically targeted animal. Panel A shows DAPI stain, Panel B shows GFP; Panel C shown alpha-sarcomeric actin; Panel D shows SPM beads; Panel E shows GFP and alpha-sarcomeric actin colocalization; Panel F shows GFP and SPM colocalization. Solid white arrows indicate SPM positive/GFP positive cells which were detected in the peri-infarct region of the heart, which indicates that remnant SPMs in the cytoplasm did not prevent CDCs from differentiating into cardiomyocyte phenotypes. Empty white arrowheads depict cells that are SPM negative, but GFP and alpha-sarcomeric actin positive cells, indicating that some CDCs exocytosed the SPMs.

FIGS. 25A-25D depict fluorescent confocal images of endothelial proteins in GFP positive cells (CDCs). Panel A shows DAPI staining; Panel B shows staining for the endothelial protein, von Willebrand Factor; Panel C shows GFP (CDCs); and Panel D shows the merged figures. Colocalization of GFP positive vWF positive cells suggests that transplanted CDCs participated in regeneration of vascular structures through differentiation into endothelial phenotypes.

FIG. 26 depicts a general design of an intracoronary efficacy study described and utilized herein.

FIGS. 27A-27E depict SPM labeling of rat CDCs. As shown in Panel A, rat CDCs were co-incubated with flash red-conjugated SPMs for 24 hours at a 500:1 SPM: cell ratio. CDCs were then examined by fluorescence microscopy. Panel B shows cells that were fixed and stained for Prussian Blue (iron) and counter-stained with nuclear red. Non-labeled cells did not express flash red fluorescence or Prussian Blue staining (Insets of A and B). Bars=50 um. Panel C shows WST-8 proliferation assay of CDCs and Fe-CDCs (n=3). No significant differences were detected. Panel D depicts a Western blot analysis of caspase-3 and Panel E depicts TUNEL staining, which revealed no evident increase of apoptosis in the Fe-CDCs.

FIGS. 28A-28M depict dosage optimization for intracoronary infusion of Fe-CDCs with and without magnetic targeting. Animals were sacrificed 24 hours after cell infusion. Panels A-J shows increasing fluorescent imaging revealed increases in fluorescent intensity with increasing doses of un-targeted (A-E) and magnetically targeted CDCs (F-J). High density areas were seen when 1×10⁶ and 2×10⁶ cells were infused (circled with pink). Panel L depicts cell numbers per mg of heart tissue as measured by qPCR and plotted against cell dose. “*” indicates p<0.05 when compared to the Fe-CDC group. Panel M depicts C serum TnI values plotted against doses (n=3 per data point). # indicates p<0.05 when compared to the Control (dose=0) group.

FIGS. 29A-29F depict micro-embolization of CDCs at high cell infusion doses. Animals were sacrificed 24 hours after i.c. infusion and representative heart sections were stained for α-SMA to detect blood vessels. Fe-CDCs were visualized by flash red fluorescence. In panels A-D, at the infusion dose of 5×10⁵ Fe-CDCs, blood vessels containing cells were readily detected and the vessels were still patent. However, at the dose of 1×10⁶ Fe-CDCs, many blood vessels were blocked by cell clumps. Panel E depicts quantification of blocked vessels. Panel F depicts quantification of unblocked vessels that contain cells. n=3 animals per group. Bar=50 um.

FIGS. 30A-30F depict the effects of magnetic application duration on cell retention. Animals were i.c. infused with 500,000 Fe-CDCs with various times of magnet application. 24 hours later, animals were sacrificed and hearts were excised for fluorescent imaging of retained Fe-CDCs. Panel A depicts representative fluorescent imaging images of the hearts with various time of magnetic targeting. Panel F depicts quantification of fluorescent intensities (indicator of Fe-CDCs).

FIGS. 31A-31B depict the effects of magnetic targeting on short-term cell retention and long-term cell engraftment. Panel A depicts female animals (n=5) were sacrificed 24 hours after cell injection. Donor male cells that were retained in the female hearts were detected by quantitative PCR for the SRY gene. Panel B, similar PCR experiment performed 3 weeks after injection to quantify engraftment.

FIGS. 32A-32C depicts transvascular relocation of i.c. infused Fe-CDCs 72 hours after delivery. Ischemia/reperfusion animals that received 500,000 Fe-CDCs with and without magnetic enhancement were sacrificed 72 hours after cell infusion (n=3 per group). Representative heart sections were stained for aSMA for blood vessels. Fe-CDCs were visualized with flash red fluorescence. Panels A and B depict representative confocal images from the Fe-CDC+magnet and Fe-CDC group. Panel C depicts quantification of Fe-CDCs per high power field. Bar=50 um.

FIGS. 33A-33G depict morphometric heart analysis. Panel A shows representative Masson's trichrome-stained myocardial sections 3 weeks after treatment for the indicated treatment groups (n=7 per group). Scar tissue and viable myocardium are identified by blue and red color, respectively. Panels B-E depict various quantitative analysis endpoints and LV morphometric parameters. A “*” indicates P<0.05 when compared to Control and “#” indicates P<0.05 when compared to the Fe-CDC group.

FIGS. 34A-34H depict the enhancement of functional benefit via the magnetic targeting of i.c. delivered of Fe-CDCs. Panels A-F depicts representative long-axis diastolic and systolic images at 3 weeks after treatment for the indicated groups. Panel G depicts the left ventricular ejection fraction (LVEF) measured by echocardiography at baseline and 3 weeks after cell administration (n=9 per group). Baseline LVEFs were indistinguishable among the 3 groups. Panel H depicts the changes of LVEF from baseline to 3 weeks in each group. Values are expressed as mean±S.D.

FIGS. 35A-35D depict improved engraftment and cardiac differentiation of infused CDCs. Animals were sacrificed 3 weeks after cell infusion (n=5 animals per group) and representative heart sections were stained for DAPI, GFP, α-sarcomeric actin (aSA). Panels A & B depict representative confocal images from the Fe-CDC and Fe-CDC+magnet groups, respectively. The Fe-CDC+magnet group had more GFP^{POS} and GFP^{POS}/αSA^{POS} cells. This indicated that magnetic targeting improved long-term cell engraftment. Panel C depicts quantification of GFP^{POS} cells in the risk and normal region. Panel D depicts quantification of GFP^{POS}/αSA^{POS} cells. Bar=100 um.

FIGS. 36A-36E depict expression of cardiac markers in SPM^{POS}/GFP^{POS} cells. through representative confocal images showing colocalization of GFP with alpha-sarcomeric actin (alpha-SA) in the peri-infarct region of a Fe-CDC+Magnet animal. Panel A shows the DAPI stain; panel B shows alpha-SA; panel C shows GFP; Panel D shows SPM; and Panel E shows the merged channels. SPM^{POS}/GFP^{POS}/alpha-SA^{POS} cells (white arrows) were detected in the region, indicating that remnant SPMs in the cytoplasm did not prevent CDCs from differentiating into a cardiomyocyte phenotype. SPM^{NEG}/GFP^{POS}/alpha-SA^{POS} cells were also detected (blue arrowheads), indicative of cells that exocytosed the SPMs before or after acquiring a cardiomyocyte phenotype. Bars=50 μm.

FIGS. 37A-D depicts a representative confocal image showing colocalization of GFP with von Willebrand factor (vWF) in an arteriole in the peri-infarct region of a Fe-CDC+Magnet animal. Panel A shows DAPI; panel B shows vWF; panel C shows GFP; and panel D shows merged images. GFP^{POS}/vWF^{POS} cells are indicated by white arrows. The colocalization of GFP with vWF suggests that transplanted CDCs participated in regeneration of vascular structures, differentiating into an endothelial phenotype. Bars=50 μm.

FIGS. 38A-38D depict tissue density of CD68 positive macrophages. Representative confocal images showing the presence of CD68POS macrophages from hearts excised at 3 weeks from the PBS control (A), Fe-CDC (B) and Fe-CDC+magnet (C) groups. Co-localization of CD68 with SPM (C inset; solid arrowheads) as detected in the sections from the Fe-CDC and Fe-CDC+magnet group. Panel D depicts quantification of total CD68^{POS} macrophages per low power field (n=5 animals per group). The values from all three treatment groups were indistinguishable. Bar=50 um.

FIGS. 39A-39C depict Ki67-positive cardiomyocytes. Animals were sacrificed 3 weeks after cell infusion (n=5 animals per group) and representative heart sections were stained for DAPI, Ki67, and α-sarcomeric actin (aSA). Panels A & B are representative confocal images from the Fe-CDC and Fe-CDC+magnet group. The Fe-CDC+magnet group had more Ki67^{POS}/αSA^{POS} cells (green arrows and insets), indicating more cardiomyocytes were proliferative or newly formed. Panel C depicts quantification of Ki67^{POS}/αSA^{POS} cells. Bars=100 um.

FIGS. 40A-40C depict recruitment of endogenous c-kit positive cells. Representative confocal images are shown that indicate the presence of endogenous (GFP^{NEG}/c-kit^{POS}; arrows) and exogenous GFP^{POS}/c-kit^{POS}; arrowheads) cells from the hearts excised at 3 weeks from Fe-CDC+magnet (Panel A) and Fe-CDC (Panel B) groups. Figure C depicts the quantification of endogenous GFP^{NEG}/c-kit^{POS} cells per high power field (n=5 animals per group). Bar=50 um.

FIGS. 41A-41D depict tissue preservation due to magnetic targeting. Representative confocal images are shown that indicate the presence of TUNEL^{POS} apoptotic cells from the hearts excised at 3 weeks from Fe-CDC+magnet (Panel A), Fe-CDC (Panel B), and PBS control (Panel C) groups. Panel D shows quantification of TUNEL^{POS} cells per high power field (n=5 animals per group). Bar=50 um. * indicates p<0.05 when compared to PBS control; # indicated p<0.05 when compared to Fe-CDC.

FIGS. 42A-42B depict serum levels of transferrin (Panel A) and ferritin (Panel B) for each of the treatment groups.

FIGS. 43A-43J depict the lack of off-target accumulation of SPM-labeled cells. Prussian Blue staining revealed no evident iron clusters from the lungs, livers, and spleens in all three groups. In contrast, a positive control (direct injection of a spleen with Fe-CDCs followed by immediate cryo-sectioning) indicates clear Prussian Blue signal (Panel J).

### DETAILED DESCRIPTION

The compositions and methods provided herein are useful for facilitating delivery of cells to target tissues or organs and improving the retention rate and/or engraftment of administered cells. Specifically, the compositions and methods provided herein are useful for localizing cells, including stem cells, such as CDCs, to a cardiac tissue and improving the typically low retention rate of delivered cells due to the wash-out effect caused by blood flow and the contraction of the heart. See, for example Table 1, which depicts retention rates from various delivery methods using various non-targeted cell types.

TABLE 1
Cell Retention Rates

| Cell Retention | | | | Delivery | Animal |
|---|---|---|---|---|---|
| Rate (%) | Time Point | | Cell Type | Method | model |
| 11.1 | 10 | min | Microspheres | IM | Pig |
| 17.6 | 1 | hr | CDCs | IM | Rat |
| 5.5 | 1 | hr | CD34+ cells | IC | Human |
| 11 | 1 | hr | PMNCs | IM | Pig |
| 2.6 | 1 | hr | PMNCs | IC | Pig |
| 3.2 | 1 | hr | PMNCs | RCV | Pig |
| 1.3 to 2.6 | 75 | min | BM stem cells | IC | Human |
| 2.03 | 24 | hr | EPCs | IV | Rat |
| 4.70 | 24 | hr | EPCs | ILV | Rat |
| <1 | 24 | hr | CDCs | IC | Pig |
| 8 | 24 | hr | CDCs | IM | Pig |

IM—intramyocardial;
IC—intracoronary,
RCV—retrograde coronary venous,
IV—intravenous;
ILV—intra left ventricular cavity.

As such, the compositions and methods provided herein can be used to treat a variety of heart diseases or disorders. The compositions and methods provided herein can also be used for the treatment of other diseases or disorders, for example, hepatic diseases, cancers, neurodegenerative diseases or diabetes, and diseases involving digestive and urogenital systems, among others.

### Stem Cells or Other Types of Cells

Cells useful in the compositions and methods provided herein include any type of cells known in the art, for example, CDCs or endothelial cells. The cells as used herein can, for example, be genetically modified or otherwise modified, and can be either free or encapsulated in a matrix. In some embodiments, the cells as used herein are stem cells, such as cardiac-derived stem cells or CDCs.

### Stem Cells

In several embodiments, stem cells useful for the compositions and methods provided herein include those listed in Table 2. Stem cells useful for the compositions and methods provided herein can include embryonic or adult stem cells. Such stem cells can include, for example, embryonic stem cells, amniotic stem cells, bone marrow stem cells, placenta-derived stem cells, embryonic germ cells, cardiac stem cells, CDCs, induced pluripotent stem cells, mesenchymal stem cells, endothelial progenitor cells, adipose-derived stem cells, cord blood stem cells, and spermatocytes. The stem cells employed can be autologous or heterologous to the subject being treated. In some embodiments, the stem cells are autologous stem cells.

TABLE 2
Examples of Stem Cells and Sources

| Cell Type | Representative Source |
|---|---|
| Embryonic stem cells | Embryo |
| Amniotic stem cells | Placenta |
| Mesenchymal stem cells | Marrow, fat |
| Endothelial progenitor cells | Marrow, blood |
| Cardiac stem cells | Cardiac biopsy |
| Cardiosphere-derived stem cells | Cardiac biopsy |
| Skeletal myoblast | Muscle biopsy |
| Adult spermatocytes | Testicular biopsy |
| Induced pluripotent stem cells | Adult somatic cells, including skin |

In several embodiments, the stem cells can be a homogeneous composition while in some embodiments the stem cells can be a mixed cell population, for example, enriched with a particular type of stem cell. Homogeneous cell compositions can be obtained, for example, by cell surface markers characteristic of stem cells, or particular types of stem cells, in conjunction with monoclonal antibodies directed to the specific cell surface markers. Homogenous cell compositions, for example, those comprising CDCs, can also be obtained without the use of antibody reagents for selection using standard techniques (see, e.g., Smith et al. (2007) Circulation 115:896), which is incorporated by reference herein.

In some embodiments, the stem cells are CDCs. The cells that form the cardiospheres can, for example, be obtained from cardiac surgical biopsy specimens taken from a subject, such as a human (e.g., a human with acute or chronic heart failure or other cardiac injury). In some embodiments, the specimen samples are obtained by a non-invasive method, for instance, by a simple percutaneous entry. The cardiospheres can be disaggregated using standard means known in the art for separating cell clumps or aggregates, for example, agitation, shaking, blending, or in some embodiments, enzymatic digestion. In some embodiments, the cardiospheres are disaggregated to single cells. In other embodiments, the cardiospheres are disaggregated to smaller aggregates of cells. In some embodiments, after disaggregation, the cells are grown on a solid surface (e.g., glass or plastic), such as a culture dish, a vessel wall or bottom, a micro titer dish, a bead, flask, or roller bottle. The cells adhere to the material of the solid surface or, in some embodiments, the solid surface is coated with a substance that encourages adherence. Such substances are well known in the art and include, for example, fibronectin, hydrogels, polymers, laminin, serum, collagen, gelatin, and poly-L-lysine. In some embodiments, growth on the surface will be monolayer growth.

After surface growth, in several embodiments the disaggregated cells are grown under conditions which favor formation of cardiospheres. Repeated cycling between surface growth and suspension growth (cardiospheres) leads to a rapid and exponential expansion of desired cells. In some embodiments, the cardiosphere phase is eliminated, and instead the cells are surface expanded, e.g., repeatedly surface expanded, without the formation of cardiospheres. The culturing of CDCs, whether on cell surfaces or in cardiospheres, is performed in the absence of exogenous growth factors in some embodiments. While fetal bovine serum can be used, under growth conditions of some embodiments described herein, other factors are expendable, such as EGF, bFGF, cardiotrophin-1, and thrombin. More information regarding the preparation and culture of CDCs can be found, for example, in U.S. Pub. No. 2008/0267921, which is incorporated by reference herein.

The stem cells can be obtained or derived from any of a variety of sources. For example, subjects that can be the donors (or recipients) of stem cells in the methods presented herein include, for example, mammals, such as non-primates (e.g., cows, pigs, horses, cats, dogs; rats or rabbits) or primates (e.g., monkeys or humans). In some embodiments, the subject is a human. In one embodiment, the subject is a mammal, e.g., a human, such as a human with acute or chronic heart failure or other cardiac tissue injury.

While a single species or individual can be the donor by providing the cells and be the recipient by receiving the cells (i.e., autologous stem cells), in some embodiments the donor and recipient of the stem cells may be of different species (i.e., xenogeneic). For instance, porcine cells can be administered into human cardiac tissue. In some embodiments, the stem cells are allogeneic or syngeneic. In some embodiments, the stem cells are autologous to the cardiac tissue. Having an autologous source of stem cells from the same individual further decreases the possibility of avoiding transplant rejection such as Graft-versus-Host Disease (GVHD). In some embodiments, the autologous stem cells are derived from adult non-cardiac tissue. In some embodiments, the stem cells are induced pluripotent stem cells derived or created from somatic adult cells, e.g., dermal fibroblasts, using techniques known in the art (see, e.g., Takahashi et al. (2007) Cell 131:861; Yu et al. (2007) Science 318:1917), herein incorporated by reference.

### Other Types of Cells

In some embodiments, cell types other than stem cells can be used in the compositions and methods provided herein. Choice of a particular cell type can be determined by the particular tissue or organ for which delivery or treatment is desired.

In some embodiments, the tissue or organ is or is part of the lymphatic system, liver, spleen, pancreas, heart, urogenital tract, gastrointestinal tract, respiratory system, portal venous system, ventricular fluid system, or cerebrospinal fluid system. In some embodiments, the tissue or organ for which delivery or treatment is desired comprises cancerous areas, areas of atherosclerosis, areas of post-angioplasty restenosis, areas of plaque fracture, sites of thrombosis or sites of vasculitis. In some embodiments, the tissue or organ comprises gravity-dependent and gravity-independent areas. In some embodiments, the target tissue or organ comprises a luminal surface.

In one embodiment, magnetic-particle labeled cardiac cells, endothelial cells, fibroblasts or smooth muscle cells are administered to the heart, e.g., to diseased or injured cardiac tissue. In some embodiments, magnetic particle-labeled endothelial cells, fibroblasts or hepatocytes can be delivered to the liver to treat hepatic disease. In one embodiment, magnetic particle-labeled neural cells, neuroglial cells, endothelial cells or fibroblasts can be delivered to the brain or spinal cord. In one embodiment, magnetic particle-labeled endothelial cells, fibroblasts, pancreatic islet cells or other pancreatic cells can be administered to the pancreas. In some embodiments, magnetic particle-labeled endothelial cells, fibroblasts or respiratory epithelial cells can be administered to the lung or respiratory system. In one embodiment, magnetic particle-labeled endothelial cells, smooth muscle cells or fibroblasts can be administered to blood vessels, e.g., atherosclerotic vessels. In another embodiment, magnetic particle-labeled endothelial cells, epithelial cells, fibroblasts or smooth muscle cells can be administered to gastrointestinal or urogenital tissues.

### Methods of Labeling Cells

Magnetic particles as used in the compositions and methods provided herein can be in any forms known in the art, e.g., fluid (e.g., ferrofluid), microspheres, conjugates (e.g., poly-L-Lysine (“PLL”) conjugates), micelles, colloids, liposomes, aggregates, or complexes with a range of various sizes. In some embodiments, the diameter of a magnetic particle as used herein ranges from about 10 nm to about 20000 nm, from about 500 nm to about 7000 nm, from about 1000 nm to about 6000 nm, or from about 3000 nm to about 5000 nm, from about 300 nm to about 900 nm, or from about 50 nm to about 500 nm, and overlapping ranges thereof. In several embodiments, the diameter of a magnetic particle as used herein is about 900 nm. Any material that is responsive to a magnetic field can be used, for example, a ferromagnetic, paramagnetic or superparamagnetic material (see, e.g., Thorek et al. (2006) Annals of Biomedical Engineering, 34(1):23-28; herein incorporated by reference). In some embodiments, the magnetic particles are naturally occurring proteins, for instance, ferritin conjugates. In some embodiments, the magnetic particles are nanoparticles, e.g., superparamagnetic iron oxide (SPIO). In some embodiments, the magnetic particles are biodegradable, e.g., biodegradable magnetic microspheres. Magnetic particles as used herein can be obtained, for example, by spray drying magnetic material under gravity, or under the presence of an applied electric or magnetic field. Magnetic particles useful in the methods provided herein are also commercially available (e.g., Endorem, or Amag Pharmaceutical Inc.'s FERIDEX®, FERIDEX IV®).

In several embodiments, cells (e.g., stem cells, such as CDCs) used in the compositions and methods provided herein are labeled with magnetic particles. Labeling may be accomplished by any known technique in the art (see, for example, Yeh et al. (1993) Magnetic Resonance in Medicine 30(5):617-625; herein incorporated by reference). In some embodiments, magnetic particles are incorporated into cells by culturing the cells under conditions in which the magnetic particles are internalized (e.g., endocytosis or phagocytosis). In some embodiments, magnetic particles remain extracellular (e.g., via antibody-conjugated beads). In still other embodiments, combinations of internal and external labeling are used. In some embodiments, magnetic particles are incorporated into cells by incubation of cells in a growth medium containing magnetic particles. The incubation time and the ratio of magnetic particles to cells can vary depending on the cell type used. In some embodiments, the cells as used herein are incubated in a magnetic-particle containing medium overnight, or for about 2, 4, 6, 10, 12, 20, 24, or 30 hours, or for about 1, about 3, about 5, about 6, or about 7 hours, and overlapping ranges thereof. In some embodiments, CDCs are incubated with SPIO-containing medium overnight. In some embodiments, the magnetic particle-to-cell ratio for cell labeling is about 4000:1, about 2000:1; about 1000:1, about 750:1, about 500:1, about 250:1, about 100:1, about 50:1, about 25:1, about 10:1, about 5:1, or about 2:1. In some embodiments, CDCs are labeled with SPIO microspheres at a 500:1 SPIO-to-cell ratio.

In some embodiments, magnetic particles can be introduced to the cells via one or a combination of selected vehicles or carriers, e.g., liposomes containing magnetic particles (“magnetoliposomes”) or other transfection agents (e.g., PLL, Sigma, Sigma, St. Louis, Mo.). In still other embodiments, magnetic particles can be produced by introducing exogenous genes expressing magnetic particles. Further methods of obtaining magnetic particles include producing magnetic particles (e.g., magnetosomes) from magnetic microorganisms (e.g., bacterium Magnetospirillum spec. ABMI (JP7-241192-A) or bacterium Magnetospirillum gryphiswaldense). Information related to obtaining magnetic particles from microorganisms can be found, for instance, in U.S. Pat. No. 6,251,365 and US 2002/0012698, herein incorporated by reference.

In other embodiments, magnetic particles are directly or indirectly coupled to certain extra-cellular matrix components (e.g., collagen, fibronectin,) or specific chemical agents to facilitate organ- or tissue-specific endocytosis or binding.

In some embodiments, cells can be labeled by magnetic particles that are directly or indirectly coupled to specific antibodies, or antibody-conjugated beads. In some embodiments, the antibody-conjugated beads also allow for enrichment of selective cell populations. For example, in some embodiments, the antibodies on the magnetic particles can recognize specific antigens on the cell surface (e.g., c-kit, CD105, CD90 or CD31) and thus render the specific cell population magnetically responsive. In some embodiments, the antibodies on the magnetic particles can recognize integrin, fibronectin, and/or tissue factor(s). In some embodiments, antibodies on the magnetic particles that recognize integrin, fibronectin, and/or tissue factor(s) may have the capacity to target more cell types and allow selection of cell populations with lower specificity.

In several embodiments, a magnetic particle can be linked to two antibodies. In some embodiments, a first antibody linked to the particle can be used to link the particle to a particular therapeutic cell type, for example a CDC, based on known antigens expressed on the surface of that cell type. In some embodiments, a first antibody directed to c-kit can be used to selectively enrich a c-kit positive population of stem cells, Other markers, such a CD-105, CD-90, or CD 31 can also be used. In some embodiments, antibodies can be directed against an antigen that is genetically manipulated. For example, a non-native antigen may be engineered to be expressed on a therapeutic cell, for example a liver-specific marker on a non-liver cell type. In this manner, a particular population of cells known to be genetically modified may be selectively enriched. A second antibody linked to the magnetic particle could be directed to a known antigen on a desired target tissue. For example, in some embodiments, the second antibody could recognize a cardiac tissue specific marker. In some embodiments, a cardiac specific maker that is upregulated in response to injury may be chosen. For example, proinflammatory cytokines such as IL-1beta and IL-6 are upregulated shortly after myocardial infarction. As such, the second antibody can be used to selectively target damaged myocardial tissue. In still additional embodiments, apoptotic surface markers, such as Fas (e.g. CD 95) could be targeted with the antibody. Thus, in some embodiments, the combination of antigenic selection and magnetic targeting could be used to accomplish one or more of: selective enrichment of therapeutic cells, selective targeting of specific target cells based on antigen expression, and magnetic enhancement of therapeutic cell retention at the specific target cells. In some embodiments, the efficiency of the therapeutic cells may be enhanced, by improving the delivery, retention, and or engraftment of the cells bound to the particle via the antibody. In some embodiments, single bi-functional antibodies linked to a magnetic particle can be used in place of two distinct antibodies. In still additional embodiments, drugs or other pharmaceuticals may be used in place of therapeutic cells. In still further embodiments, the magnetic particles are also used for visualization of the therapeutic cells or agents, while in other embodiments, the magnetic particles are not visualized.

Magnetic particles useful in the methods provided herein are also commercially available (e.g., MACS® MicroBeads; Miltenyi Biotec Inc., Auburn, Calif.). In some embodiments, cell labeling can take place prior to administration of cells to the target tissue or organ, while in other embodiments labeling is performed concurrently with administration of cells to the target tissue or organ.

In some embodiments, the magnetic particles are also useful for monitoring labeled cells administered to the tissue or organ using magnetic resonance imaging (MRI). By way of illustration only, the magnetic particles (e.g., SPIO) can lead to a marked decrease in the MRI parameter T2* and offer the possibility of non-invasive in vivo tracking of the labeled cells. However, in some embodiments, the magnetic particles are not used for imaging or visualization purposes.

In some embodiments, magnetic particles can be integrated or encapsulated into liposomes (e.g., ferrofluid) prior to being incorporated into cells. In such embodiments, the liposomes containing magnetic particles can be referred to as magnetoliposomes, or magnetite cationic liposomes (MCLs). By way of illustration only, magnetoliposomes can be used as carriers to introduce magnetite nanoparticles into target cells as their positively charged surface interacts with the negatively charged cell surface.

Suitable liposomes for use in preparing magnetoliposomes provided herein include, but are not limited to, classical liposomes (MLV, SUV, LUV); stealth liposomes (PEG); micellar systems (e.g., SDS, triton, sodium cholate); immunoliposomes containing antibodies or Fab fragments against antigens associated with diseases or adhesion molecules bound to the surface of the liposomes; cationic liposomes (DAC-Chol, DOC-SPER); and fusogenic liposomes (reconstituted fusion proteins in liposomes). The choice of a particular type of liposome(s) will depend on, for example, the type of cells being used and the particular tissue or organ for which treatment is desired.

Magnetoliposomes suitable for use with the methods provided herein can be prepared according to methods known in the art, for example, as described in German patents Nos. 4134158, 4430593, 4446937 and 19631189, herein incorporated by reference.

In some embodiments, the magnetoliposomes are used to monitor the magnetic-labeled cells administered to the tissue or organ using MRI. For example, by creating magnetic field in homogeneity, magnetic particles (e.g., ferrofluid) permit local contrast enhancement in magnetic resonance imaging and thereby offers the potential to monitor magnetoliposomes in vivo. The response of magnetic particle-containing magnetoliposomes depends on the nature of the magnetic field.

In some embodiments, the cell labeling process takes place ex vivo. In other embodiments, the cell labeling process occurs in vivo. For example, magnetic particles alone can also, in some embodiments, be infused into the body and subsequently label subpopulations of the host cells by endocytosis or cell surface binding. In some embodiments, magnetic particles can be coupled with antibodies that recognize specific cell populations in the body.

The efficiency of cell labeling as provided herein can be assessed by any of a variety of methods known in the art. In some embodiments, the labeling efficiency is assessed by microscopic examination. In other embodiments, the labeling efficiency is assessed by flow cytometry. Alternatively, in some embodiments, the labeling efficiency can be assessed by a magnetometer, for example, a superconducting quantum interference device (SQUID), to measure the amount of magnetic particles (e.g., iron oxide) in the cells. The magnetic properties of labeled CDCs can be assessed, in some embodiments, for example, by MRI. In some embodiments, images of labeled cells in the culture plate under which the magnet is placed can be compared with labeled cells without magnet attraction.

In several embodiments, labeling process has limited adverse effects on the cells. For example, in some, cell viability is substantially maintained during and after the labeling protocol. In some embodiments, labeling does not affect the do not affect the antigenic phenotype or proliferation of the labeled cells. In some embodiments, modest increases in apoptosis of cells occur during the labeling process, however, in some such embodiments, necrosis of the cells is lessened. As such, a healthy and viable labeled cell population is produced according to several of the labeling embodiments described herein. In some embodiments, the magnetic particles are used not only to label and direct the cells to a target, but are also used in imaging or visualization (e.g., to confirm targeted delivery). In some embodiments, imaging of the particles is not performed.

### Methods of Delivering Cells

The magnetic-particle labeled cells used in the compositions and methods provided herein can be administered to (or otherwise contacted with) a tissue or organ (e.g., heart, kidney, spinal cord or liver) by methods known in the art. The tissue or organ can be a selected tissue or organ into which local cell delivery is desired. In some embodiments, the cells are administered to (or otherwise contacted with) a cardiac tissue. In some embodiments, the cardiac tissue is an injured cardiac tissue. In some embodiments, other tissues are targeted. In some embodiments, the targeted tissue and the magnetic particle-labeled cells are of the same type (e.g., both cardiac), while in other embodiments, the cells and the target tissue are of a distinct type (e.g., bone marrow derived stem cell into the liver).

In several embodiments, the magnetic particle-labeled cells are delivered systemically to a recipient. In other embodiments, local delivery to the target tissue or organ is used. In some embodiments, the magnetic particle-labeled cells are administered to a tissue or organ before, during or after a surgery. In some embodiments, the delivery takes place while the organ is actively functioning (e.g., while the heart is beating) rather than during a period of artificial inactivity (e.g., occlusion of a blood vessel to target the vascular wall). In other embodiments, the magnetic particle-labeled cells are delivered to a tissue or organ using non-surgical methods, for example, either locally by direct injection into the selected tissues, to a remote site and allowed to passively circulate to the target site, or to a remote site and actively directed to the target site with a magnet. Such non-surgical delivery methods include, for example, infusion or intravascular (e.g., intravenous or intra-arterial), intramuscular, intraperitoneal, intrathecal, intradermal or subcutaneous administration.

In some embodiments, magnetic particle-labeled cells are administered with one or more therapeutic agents, either magnetized or unmagnetized, to a target tissue or organ. Such therapeutic agents include, for example, antineoplastic agents, angiogenic factors, or anti-angiogenic factors, immuno-suppressants, or antiproliferatives (anti-restenosis agents), embryonic factors, fibroblast growth factors, transcription factors, kinase inhibitors or adenosine. Other non-limiting examples of therapeutic agents are provided elsewhere herein. The therapeutic agents can be administered to or otherwise contacted with the cardiac tissue by any of a variety of procedures known in the art either alone or in combination with each other, and optionally in combination with the magnetic particle-labeled cells and in any sequence (i.e., concurrently or sequentially). The therapeutic drugs or agents suitable for use with the methods provided herein will often be combined with one or more physiologically acceptable carriers such as sterile water, sterile saline, e.g., isotonic saline. Other physiologically acceptable carriers are known in the field.

In some embodiments, the magnetic particle-labeled cells are administered with unlabeled cells (e.g., unlabeled endothelial cells), either concurrently or sequentially to the target tissue or organ. The unlabeled cells can be administered to the tissue or organ by any applicable methods (e.g., methods applicable for administration of labeled cells). In some embodiments, the labeled cells are delivered to the tissue or organ followed by administration of unlabeled cells. In other embodiments, the labeled cells are concurrently administered with the unlabeled cells to the tissue or organ. In still other embodiments, the labeled cells are administered to the tissue or organ after administration of unlabeled cells. In yet other embodiments, the cells are labeled concurrently with administration to the tissue or organ.

In some embodiments, the labeled cells are present in a percentage of about 10% to about 90% of the total cells to be delivered to a selected tissue or organ, including about 20% to about 80%, about 30% to about 70%, about 40% to about 60%, about 45% to about 55%, and overlapping ranges thereof. In some embodiments, wherein local delivery requires circumferential cellular coverage, the area to be covered can be placed horizontally and an external magnet can be placed above the target tissue. In some embodiments, a mixture of labeled and unlabeled cells is administered to the target tissue in order to target gravity independent and gravity dependent areas, respectively. In some embodiments, the target tissue is rotated axially to increase the gravity-dependent area and improve gravity-dependent cell coverage. Alternatively, circumferential coverage can be obtained by surrounding the target tissue with magnets and delivering only magnetic particle-containing cells.

In some embodiments, labeled cells are guided and pulled towards the target tissue or organ by one or more magnetic fields or magnetic field gradients (e.g., an external source of magnetic fields or magnetic field gradients). Such fields or gradients can be generated by, for example, one or more magnets and associated medical devices placed within or adjacent to a target tissue or organ prior to, during or after cell delivery. In some embodiments, the magnets are placed inside the body using surgical or percutaneous methods inside the target tissue, or outside the target tissue (e.g., around or adjacent to the target tissue). In some embodiments, the magnets are external magnets that are placed outside of a subject's body to create an external source of magnetic field around or adjacent to the target tissue or organ. In some embodiments, the source of magnetic fields is a permanent magnet (e.g., neodymium (NdFeB) magnet). In one embodiment, the source of magnetic fields is an electro-magnet. In other embodiments, the size of magnets ranges from about 1 mm to about 10 m and the strength of magnetic fields ranges from about 0.1 Tesla to about 100 Tesla, including about 0.1 to about 0.5 Tesla, about 0.5 to about 1 Tesla, about 1 Tesla to about 1.1 Tesla, about 1.1 Tesla to about 1.2 Tesla, about 1.2 Tesla to about 1.3 Tesla, about 1.3 Tesla to about 1.4 Tesla, about 1.4 Tesla to about 1.5 Tesla, about 1.5 Tesla to about 2 Tesla, about 2 Tesla to about 4 Tesla, about 4 Tesla to about 10 Tesla, about 10 Tesla to about 30 Tesla, about 30 Tesla to about 50 Tesla, about 50 Tesla to about 70 Tesla, about 70 Tesla to about 90 Tesla, and overlapping ranges thereof. In several embodiments, the magnetic field is applied for a time period ranging from about 1 minute up to about 5 hours. In some embodiments, the magnetic field is applied for about 1 minute to 5 minutes, about 5 minutes to about 10 minutes, about 10 minutes to about 20, about 20 minutes to about 30 minutes, and overlapping ranges thereof. In several embodiments, the magnetic field is applied for about 5-15 minutes, including about 6, 7, 8, 9, 10, 11, 12, 13, or 14 minutes.

In some embodiments, the source of magnetic fields is from one or more magnets of an apparatus (e.g., a group of magnets as an integral apparatus to shape and focus the magnetic field). Such apparatus can include, for example, a surgical tool (e.g., catheters, guidewires, and secondary tools such as lasers and balloons, biopsy needles, endoscopy probes, and similar devices) with a magnetic tip attachment (see, e.g., U.S. Pat. No. 7,280,863 and U.S. Pat. Publ. Nos. 2007/1116006, 2006/0116634, 2008/0249395, 2006/0114088 and 2004/0019447; each of which is herein incorporated by reference). Thus in some embodiments, labeled cells are delivered and an external magnet is used to target the cells (e.g., percutaneous or surgical access to the heart for cell injection with a magnet placed on the chest of a human subject). In some embodiments, labeled cells are delivered endomyocardially and a magnet is used to direct the cells into a target site of the heart (e.g., delivery of labeled cells via endomyocardial catheter with a magnet placed on the chest of a human subject). In some embodiments, labeled cells are delivered endomyocardially and a magnet associated with or integrated into the delivery device provides a magnetic field to direct the cells to a target site of the heart from within the heart (e.g., delivery of labeled cells via a magnetizable endomyocardial catheter).

In several embodiments, the delivery and targeting means are combined. For example, in some embodiments, specialized catheters, used to deliver the cells. In some embodiments, the magnetic tip provided on the catheter is used to generate a localized magnetic field that functions to enhance retention of the cells at a desired target area. In some embodiments, the catheter also comprises a screw-like tip (or other shape) that allows for reversible anchoring of the catheter in tissue at the target site. Other reversible anchors may be used, such as pinchers, retractable barbs and the like. In some embodiments, the anchored tip is advantageous because the delivery can occur while the heart is beating, and the anchored tip assists in stabilizing the tip against the moving cardiac tissue. In several embodiments, the catheter is also steerable, in order to allow navigation from a remote site to the desired region of a target tissue (e.g., from a femoral access point to the endomyocardial wall). In some embodiments, the catheter comprises a controller that allows an operator to initiate the generation of a magnetic field. In some embodiments, a specific strength of magnetic field can be generated. In some embodiments, the magnetizable portion is distinct from the delivery tip, while in other embodiments, the delivery tip is housed in or adjacent to, the magnetizable portion. In some embodiments, the generation of the magnetic field acts to push the cells into the target site (e.g., a repulsion of the magnetic cells rather than an attraction). In some embodiments, the catheter comprised a delivery lumen that is of sufficient size to allow free passage of magnetically labeled cells (or other agents) from the lumen to the target site. For example, the diameter of the delivery lumen, in some embodiments, ranges from about 25 to about 50 microns, about 50 to about 100 microns, about 100 microns, to about 200 microns, about 200 to about 300 microns, about 300 to about 400 microns, and overlapping ranges thereof. In some embodiments, the presence of the magnetic field enhances the efflux of the cells from the catheter (e.g., minimizes residual, undelivered cells).

In some embodiments, the magnetic fields function primarily to target the cells to a desired location. However, in some embodiments, the magnetic fields play ancillary roles. For example, in some embodiments, the magnetic field, in conjunction with the magnetic particles, is used for imaging or visualization of the particle. In other embodiments, however, visualization or tracking is not performed. As another example, in some embodiments, magnetic fields also inhibit the induction and or progression of apoptosis, which further increases the efficacy of the delivered labeled cells.

In some embodiments, a magnetic resonance imaging (MRI) instrument or equivalent may be used to shape or focus the magnetic field. In some embodiments, computer simulations can aid magnet designs for acquiring optimal magnetic field strength to capture magnetic particle-labeled cells. For example, fluid flow rate, cell size and iron oxide content, and distance of magnet from the vessel can be considered by solving for the Khan and Richardson hydrodynamic drag force and the attractive force from the magnet, in parabolic nonpulsatile laminar flow. In some embodiments, the magnet designs can be assessed in an in vitro flow system by placing labeled cells in peristaltic pump-driven flow. In some embodiments, focusing of the magnetic field is not performed.

In some embodiments, tools such as injection needles, balloons, catheters or other acceptable delivery devices are used to deliver labeled cells. In some embodiments, the targeting magnet is placed at desired locations via a fiberoptic tube or catheter. In some embodiments, the catheter or interventional device tip is guided or monitored by a control system (e.g., a radar-assisted system or a real-time localization system) to provide more precise localization of the administered cells (see, e.g., U.S. Pat. No. 7,280,863; herein incorporated by reference). In some embodiments, a catheter Guidance Control and Imaging (GCI) apparatus is used to position and fixate a catheter, and to view the catheters' position with the x-ray imagery overlaying the display (see e.g., PCT Publ. Nos. WO 2004/006795 and 2005/042053; herein incorporated by reference). In one embodiment, such an apparatus can include, for example, an operator control that possesses a positional relationship to the catheter tip in addition to being a model representation of the actual or physical catheter tip advancing within the patient's body. In another embodiment, the physical catheter tip (the distal end of the catheter) of such apparatus can include a permanent magnet that responds to a magnetic field generated externally to the patients body (see e.g., U.S. Pat. Publ. Nos. 2004/0019447, 2006/0114088, 2006/0116633 and 2006/0116634; herein incorporated by reference). In yet another embodiment, such apparatus can include magnetic sensors to detect the magnetic field of generated by the catheter tip. In some embodiments, each sensor transmits the field magnitude and direction to a detection unit, which filters the signals and removes other field sources. The method allows the measurements of magnitude corresponding to the catheter tip distance from the sensor and the orientation of the field showing the magnetic tip orientation (see e.g., U.S. Pat. Publ. 2008/0249395; herein incorporated by reference).

In several embodiments, the magnetic field(s) are generated transiently during and after the delivery of magnetically-labeled cells. For example, in some embodiments, a magnetic field is generated just prior to the inception of delivery of the cells and maintained for several minutes after delivery. In several embodiments, the magnetic field is maintained for about 2 to about 5 minutes, about 3 to about 6 minutes, about 4 to about 7 minutes, about 5 to about 8 minutes, about 6 to about 9 minutes, or about 7 to about 10 minutes. In some embodiments, the field(s) are maintained for about 5 minutes to about 10 minutes, about 10 minutes to about 15 minutes, about 15 minutes to about 20 minutes, about 20 minutes to about 25 minutes, and overlapping ranges thereof. Longer exposure to the magnetic field is used in embodiments wherein a larger number of cells is to be delivered and/or wherein the region of damaged tissue is particularly large.

In some embodiments, an implant is employed to facilitate delivery and retention of labeled cells in the target tissue or organ. In such embodiments, labeled cells are delivered from a catheter or an interventional device distal tip to a previously placed implant (e.g., stents). In some embodiments, the implants are labeled by application of a magnetic field sequence. In these embodiments, labeled cells can be attracted by the local magnetic domains and associated field gradients within the implant, and attach onto the tissue structures locally protruding through the stent or implant struts.

In some embodiments of the methods provided herein, the magnetic particle-labeled cells are delivered to (or otherwise contacted with) a cardiac tissue. For example, in some embodiments, the labeled cells can be delivered systemically (or locally) and targeted to the heart, including specific anatomical regions of the heart. In some embodiments, labeled cells are delivered locally and targeted to a specific region of the heart. In some embodiments, the labeled cells are directly injected epicardially into a cardiac tissue, for example, during an open chest surgery. In other embodiments, the labeled cells are delivered to a cardiac tissue using non-surgical methods (e.g., minimally invasive interventions). Such methods include, for example, intravascular (e.g., intracoronary or intravenous) or intramyocardial administration. In some embodiments, the labeled cells can be delivered to a tissue via intracoronary infusion of cells, for example, CDCs, e.g., autologous CDCs. In several embodiments, intracoronary administration, despite the high rate of vascular blood flow, yields increased retention, engraftment and functional benefits when labeled CDCs are magnetically targeted during and after intracoronary infusion. In some embodiments, cells administered to cardiac tissue using non-surgical methods can be prepared for administration by mixing, admixing, or compounding the cells within an injectable liquid suspension or any other biocompatible medium.

For intravascular approaches, in some embodiments, catheters are advanced through the vasculature and into the heart to inject the cells into the cardiac tissue from within the heart. In one embodiment, the labeled cells are administered to the cardiac tissue by intracoronary administration. In another embodiment, the cells are administered to the cardiac tissue, for example, by intravenous administration, by continuous drip or as a bolus. In yet another embodiment, the labeled cells are administered to the cardiac tissue by intramyocardial administration, for example, using a conventional intracardiac syringe or a controllable endoscopic delivery device, so long as the needle lumen or bore is of sufficient diameter that shear forces will not damage the cells. In some embodiments, the labeled cells are administered to the cardiac tissue using an endocardial approach that delivers materials into the cardiac wall from within the chamber of the heart (e.g., endomyocardial procedure).

In some embodiments of the methods provided herein, the labeled cells are administered to the peri-infarct zone of cardiac tissue that was subject to an infarction. In some embodiments, the labeled cells are administered in a system, e.g., long-term, short-term and/or controlled release system, which can improve cell engraftment and persistence. In some embodiments, the system is a matrix, such as a natural or synthetic matrix (see, e.g., Simpson et al. (2007) Stem Cells 25:2350; herein incorporated by reference). The matrix can function to hold the labeled cells in place at the site of injury by serving as scaffolding. This, in turn, can enhance the opportunity for the administered cells to proliferate, differentiate and eventually become fully developed cardiomyocytes. As a result of their localization in the myocardial environment, the cells can then be retained within and integrate into the recipient's surrounding myocardium.

In some embodiments, the labeled cells are administered in a biocompatible medium which is, or becomes a semi-solid or solid matrix in situ at the site of myocardial damage. In some embodiments, the matrix is an injectable liquid which polymerizes to a semisolid gel at the site of the damaged myocardium, such as collagen and its derivatives, polylactic acid or polygly-colic acid. In other embodiments, the matrix is one or more layers of a flexible, solid matrix that is implanted in its final form, such as impregnated fibrous matrices. The matrix can be, for example, GELFOAM® (Upjohn, Kalamazoo, Mich.) or a biologic matrix. In some embodiments, the matrix is permanent. In other embodiments, the matrix is degradable or biodegradable. In some embodiments, the labeled cells are embedded into a tissue-engineered cardiac patch containing, for example, a collagen matrix. Such a patch is then be attached or otherwise delivered to the cardiac tissue, for example, with a sealant (e.g., fibrin) (see, e.g., Simpson et al. (2007) Stem Cells 25:2350; herein incorporated by reference).

In some embodiments, the labeled cells are administered to the cardiac tissue once. In other embodiments, labeled cells are administered to cardiac tissue more than one time. In several embodiments, a series of cell administrations occurs, with monitoring of the functionality of recipient's target organ being used to determine if and when an additional administration of cells is needed. In some embodiments, the labeled cells are administered as a cell suspension in a pharmaceutically acceptable liquid medium (e.g., saline or buffer), for example, for systemic administration or local administration directly into the damaged portion of the myocardium. In some embodiments, administration is localized to the cardiac tissue.

An effective dose of labeled cells for use in the methods provided herein will vary depending on the cell type used and/or the delivery site (e.g., intracoronary or intramyocardial), and such doses can be readily determined by a physician. In some embodiments, the number of cells, such as CDCs, is in the range of 1×10⁵ to 1×10⁹. For example, labeled cardiac stem cells can be administered in a dose between about 1×10⁴ to about 1×10¹⁰, between about 1×10⁵ and 1×10⁹, 1×10⁶ and 1×10⁸, such as between 1×10⁷ and 5×10⁷, or overlapping ranges thereof. Depending on the size of the damaged region of the heart, more or less cells can be used. A larger region of damage may require a larger dose of cells, and a small region of damage may require a smaller does of cells. On the basis of body weight of the recipient, an effective dose may be between 1×10⁵ and 1×10⁷ per kg of body weight, such as between 1×10⁶ and 5×10⁶ cells per kg of body weight. Patient age, general condition, and immunological status may also be used as factors in determining the dose administered, and will be readily determined by the physician.

In some embodiments, one or more of therapeutic agents, either alone or in combination, and optionally in combination with the labeled cells, can be delivered systemically or locally to the heart. For example, in some embodiments, a therapeutic agent can be administered to an injured cardiac tissue prior to administration of the labeled cells. In such embodiments, a therapeutic agent (e.g., a factor that reduces inflammation) can be administered to the injured cardiac tissue within 2, 4, 6, 10, 12 or 20 hours, or about 1, about 2, about 3, about 4, about 5, about 6 or about 7 days of the injury, e.g., an infarction. In such embodiments, labeled cells are then subsequently administered to the injured cardiac tissue, e.g., within 1, 5, 10, 15, 20, 30, 45 minutes or about 1, 2, 4, 6, 10, 12, 18, 20 or 24 hours of administration of the therapeutic agent. In one particular embodiment, such a method comprises administering an inflammation-reducing factor at least between about 3 and about 7 days post-injury (e.g., infarct), and administering labeled cells about 3, about 4, about 5 or about 6 days post-injury. In other embodiments, a therapeutic agent is administered concurrently with the labeled cells. In such embodiments, the labeled cells are optionally prepared for administration in the same carrier as the therapeutic agent. In some embodiments, the labeled cells are administered to a cardiac tissue prior to administration of a therapeutic agent. In some embodiments, therapeutic agents are optionally labeled with magnetic particles to enhance their targeting.

In some embodiments, the methods provided herein are used in combination with an agent or other type of intervention that temporarily or permanently reduces blood flow to or through the target area (e.g., an embolic). In other embodiments, the methods provided herein can be used in combination with an agent or other type of intervention that lowers the heart rate and/or cardiac contractility. In some embodiments, the agent is adenosine (e.g., 1 mg of adenosine within 1, 2, 5, 10, 15, 30, 45 or 60 min of cell delivery), verapamil, a beta-adrenergic blocker (e.g., propanolol, atenolol), a muscarinic agonist (e.g., methacholine) or combinations thereof. In other embodiments, cardiac contraction is suppressed with an agent that uncouples excitation and contraction, e.g., 2,3-butanedione-2-monoxime (BDM). In other embodiments, the injection site is “sealed” with, for example, a fibrin glue (FO) (e.g., a solution of a thrombin/calcium chloride and a fibronectin/aprotinin mixed immediately before application). However, in some embodiments, no alterations (pharmacologic or physical) are made in order to affect the heart rate or contractility of a subject's heart.

In several embodiments, such intervention can be used to counteract the effect of blood flow washing the delivered cells out of the target site. Thus, such embodiments improve cell retention or engraftment rates in the target tissue or organ. In some embodiments, administration of agents that slow ventricular rate can improve cell retention. In another embodiment, the therapeutic agents as used herein can be hydrogel such as fibrin glue. In some embodiments, co-administration of hydrogel such as fibrin glue also can improve retention by preventing cell washout. See, for example, Terrovitis et al., (2009) Journal of the American College of Cardiology Vol. 54(17)1619-1626, herein incorporated by reference.

In other embodiments of the methods provided herein, magnetic particle-labeled cells are delivered to a tissue or organ other than cardiac tissue.

In one embodiment, the magnetic particle-labeled cells are delivered to the liver directly, or to the hepatic artery or the portal-venous system to the liver. In another embodiment, the magnetic particle-labeled cells are delivered to the central nervous system via the brain, spinal cord, cerebrospinal fluid system, or circulatory system. In yet another embodiment, the magnetic particle-labeled cells are delivered to the pancreas via direct injection to the organ or injection into arteries, veins or lymphatics supplying the pancreas. In some embodiments, the magnetic particle-labeled cells are delivered to lung, or into airways, arteries, veins or lymphatics supplying the lungs to the respiratory tissues. In some embodiments, the magnetic particle-labeled cells are directly injected into the gastrointestinal tract, or via arteries, veins or lymphatics to the gastrointestinal tract. In yet another embodiment, the magnetic particle-labeled cells are administered to the urogenital system via direct injection into the urogenital tract, or via arteries, veins or lymphatics that supply the urogenital system.

### Compositions and Methods of Using Magnetic-Particle Labeled Cells to Treat Cancer

In several embodiments, magnetic particle-labeled cells can also be delivered to a tumor or a cancerous tissue as a tumor-killing tool. In some embodiments, provided herein is a method of treating or otherwise managing a cancer or tumor, comprising: (a) labeling anti-tumor cells with magnetic particles; (b) contacting the cells with the cancer or tumor; and (c) applying a magnetic field around or adjacent to the cancer or tumor. In some embodiments, magnetic field is an external magnet. In some embodiments, the magnet is placed within or adjacent to the tumor. In some embodiments, the anti-tumor cell is a T cell, such as a CD8⁺ or CD4⁺ T cell, or a natural killer (NK) cell. Other embodiments provided herein can be used in combination with embolization, chemoembolization and/or chemotherapy. In some embodiments, the anti-tumor cells can be enriched with appropriate antibody-coated magnetic beads and attracted by an external magnet or a magnet inside the tumor. In some embodiments, the antitumor cell is contacted or otherwise administered concurrently or sequentially in combination with one or more additional therapies, such as a therapeutic agent and/or a vascular permeability agent.

Non-limiting examples of tumors or cancers which can be treated in accordance with the compositions and methods provided herein include, e.g., tumors or cancers of the kidney, lung, prostate, pancreas, stomach, colon, liver, brain, testes or ovaries, oropharynx, and bladder and can be benign or malignant. Representative examples of tumors or cancers include hepatocellular adenoma, cavernous haemangioma, focal nodular hyperplasia, bile duct adenomas, bile duct cystadenomas, fibromas, lipomas, leiomyomas, mesotheliomas, teratomas, myxomas, and nodular regenerative hyperplasia, hepatocellularcarcinoma, cholangiocarcinoma, angiosarcoma, cystadenocarcinoma, squamous cell carcinoma, hepatoblastoma, melanoma, Hodgkin's and non-Hodgkin's lymphoma, tumors of the breast, ovary, and prostate.

Other non-limiting examples of tumors or cancer that can be treated by the compositions and methods provided herein include acute lymphoblastic leukemia, acute myeloid leukemia, Ewing's sarcoma, gestational trophoblastic carcinoma, Hodgkin's disease, Burkitt's lymphoma diffuse large cell lymphoma, follicular mixed lymphoma, lymphoblastic lymphoma, rhabdomyosarcoma, testicular carcinoma, wilms's tumor, anal carcinoma, bladder carcinoma breast carcinoma, chronic lymphocytic leukemia, chronic myelogenous leukemia, hairy cell leukemia, head and neck carcinoma, lung (small cell) carcinoma, multiple myeloma, follicular lymphoma, ovarian carcinoma, brain tumors (astrocytoma), cervical carcinoma, colorectal carcinoma, hepatocellular carcinoma, Kaposi's sarcoma, lung (non-small-cell) carcinoma, melanoma, pancreatic carcinoma, prostate carcinoma, soft tissue sarcoma, breast carcinoma, colorectal carcinoma (stage III), osteogenic sarcoma, ovarian carcinoma (stage III), testicular carcinoma, or combinations thereof.

### Compositions and Methods of Using Magnetic Particle-Labeled Cells to Treat Heart Diseases

In some embodiments, the compositions and methods provided herein employ magnetic particle-labeled cells and are useful for treating an injured cardiac tissue in a subject by reducing or ameliorating the progression, severity or duration of a cardiac tissue injury or a symptom thereof. In some embodiments, treatment preserves the injured cardiac tissue and function thereof, such as by preserving or reducing cell apoptosis, or by reducing cell inflammation. In other embodiments, treatment regenerates cardiac tissue, e.g., cardiac muscle or cardiac vasculature. In some embodiments, treatment activates or enhances cell proliferation or cell migration. In some embodiments, treatment increases blood flow to the injured tissue. In some embodiments, treatment increases myocardial perfusion. In some embodiments, treatment regenerates new cardiac tissue. In other embodiments, treatment increases cardiac muscle mass. In several embodiments, two or more of the above-mentioned functional or physiological parameters are improved.

In some embodiments, treatment improves global cardiac function. In some embodiments, improvements in global cardiac function are measured by, for example, stroke volume, ejection fraction, cardiac contractility and/or cardiac output using any method known in the art. In some embodiments, improving global cardiac function comprises increasing cardiac output. In some embodiments, improving global cardiac function comprises increasing ejection fraction (i.e., the fraction of blood pumped out of a ventricle with each heart beat) by at least an absolute range of about 5% to about 25%, about 5% to about 10%, about 5% to about 15%; about 5% to about 20%, about 10% to about 15%, about 10% to about 20%, about 10% to about 25%, about 15% to about 20%, about 15% to about 25%, about 20% to about 25%, and overlapping ranges thereof. Ejection fraction can be assessed by a number of methods known in the art. In some embodiments, the ejection fraction is determined by echocardiography, cardiac MRI, fast scan cardiac computed axial tomography imaging, or ventriculography. In some magnetic particle-embodiments, the ejection fraction is assessed by echocardiography.

In other embodiments, treatment improves regional cardiac function. In some embodiments, improvements in regional cardiac function are measured by wall thickening, wall motion, myocardial mass, segmental shortening, ventricular remodeling, new muscle formation, the amount of cardiac cell proliferation and programmed cell death (or their relative proportions), angiogenesis and/or the size of fibrous and infarct tissue. In some embodiments, improving regional cardiac function comprises increasing heart pumping. In some embodiments, cardiac cell proliferation is assessed by the increase in the nuclei or DNA synthesis of cardiac cells, cell cycle activities or cytokinesis. In some embodiments, programmed cell death is measured by TUNEL assay that detects DNA fragmentation. In some embodiments, programmed cell death can be assessed by measuring the expression levels of one or more genes or proteins known to be involved in the apoptotic cascade (e.g., caspases). In some embodiments, angiogenesis is detected by the increase in arteriolar and/or capillary densities. In some embodiments, cardiac function before and after treatments are assessed by echocardiography (e.g., transthoracic echocardiogram, transesophageal echocardiogram or 3D echocardiography), cardiac catheterization, magnetic resonance imaging (MRI), sonomicrometry or histological techniques. Techniques in assessing cardiac function can also be performed using methods and procedures known in the art (see, e.g., Takehara et al. (2008) J. Am. Coll. Cardiol. 52:1858-65; Laflamme et al. (2007) Nature Biotechnol. 25(9):1015-24; herein incorporated by reference).

In some embodiments, improving global cardiac function comprises increasing ejection fraction by about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%. In several embodiments ejection fraction is increased by about 2-fold, about 5-fold, or about 10-fold.

For example, in some embodiments, a patient having a tissue injury, such as a myocardial infarction, will have an ejection fraction of between about 40% to about 55% that will improve to about 66% after being subjected to a method provided herein. In some embodiments, improvements in one or more of the parameters discussed herein may or may not be associated with improvements in other parameters. For example, increases in ejection fraction, in some embodiments, may be detected despite minimal changes in cell proliferation or myocardial mass.

In some embodiments, cardiac tissue subjected to the methods provided herein has been injured, for example, due to ischemia, infarction, reperfusion or occlusion. In some embodiments, the cardiac tissue is focally injured or diseased while in other embodiments, the tissue is diffusely injured or diseased. In some embodiments, the cardiac tissue is injured as a result of acute stress, for example, acute heart failure. In other embodiments, the cardiac tissue is injured as a result of chronic stress or injury/disease, for example, chronic heart failure, systemic hypertension, pulmonary hypertension, valve dysfunction, congestive heart failure, or atheromatous disorders of blood vessels (e.g., coronary artery disease). In some embodiments, the injured cardiac tissue is in the epicardium, endocardium and/or myocardium. In some embodiments, the subject is a mammal, such as a non-primate. In some embodiments, the subject is a human. In one embodiment, the subject is a human with acute heart failure or chronic heart failure.

### Vascular Permeability Agents

In some embodiments, one or more vascular permeability agents, either alone or in combination, in combination with the magnetic particle-labeled cells, is delivered to a target tissue or organ. Generally, administration of the vascular permeability agent or agents is locally to a target tissue or organ, though in some embodiments, such agents may likewise be administered non-locally (e.g., systemically) and targeted to a desired target tissue. The term “in combination” as used herein in the context of the contacting or other administration of labeled cells and a vascular permeability agent, either alone or in combination with one or more additional therapies (e.g., an additional therapeutic agent) does not restrict the order in which agent(s) and/or cells are administered to a subject. In some embodiments, a vascular permeability agent is administered to (or contacted with) a target tissue or organ prior to administration of the labeled cells. Depending on the embodiment, the vascular permeability agent can be administered to the target tissue or organ nearly at any time point prior to administration of the labeled cells. Choice of a particular time point will depend, for example, on the time necessary to provide for good contact of agent(s) with the tissue or organ region of interest and the particular tissue or organ region for which treatment is desired. In some embodiments, a vascular permeability agent is administered about 10 seconds to about five or six hours, about 10 minutes to about two hours, or about 30 minutes to about an hour, prior to administration of the labeled cells.

In some embodiments, a vascular permeability agent is contacted or otherwise administered before (e.g., 1 minute, 5 minutes, 10 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 5 minutes, 10 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the contact or other administration of labeled cells. In some embodiments, a vascular permeability agent is administered between about 1 minute and about 60 minutes prior to administration of labeled cells. In some embodiments, a vascular permeability agent is administered concurrently with the labeled cells. In other embodiments, the labeled cells are administered to a target tissue or organ prior to administration of a vascular permeability agent.

Any cell, therapy or other agent can be contacted or otherwise administered in any order with any other cell, therapy or other agent provided herein. In some embodiments, the target tissue or organ is contacted with (e.g., by administration to the patient, target tissue or organ) (i) a vascular permeability agent and labeled cells concurrently, (ii) a vascular permeability agent followed by labeled cells, (iii) labeled cells followed by a vascular permeability agent, (iv) a therapeutic agent and labeled cells concurrently, (v) a therapeutic agent followed by labeled cells, (vi) labeled cells followed by a therapeutic agent, (vii) a vascular permeability agent, a therapeutic agent and labeled cells concurrently, (viii) a vascular permeability agent and therapeutic agent concurrently followed by labeled cells, (ix) a vascular permeability agent and labeled cells concurrently followed by a therapeutic agent; (x) a therapeutic agent and labeled cells concurrently followed by a vascular permeability agent, (xi) a vascular permeability agent followed by labeled cells and a therapeutic agent concurrently, (xii) a therapeutic agent followed by a vascular permeability agent and labeled cells concurrently, (xiii) labeled cells followed by a therapeutic agent and vascular agent concurrently; (xiv) a vascular permeability agent followed by labeled cells followed by a therapeutic agent, (xv) a vascular permeability agent followed by a therapeutic agent followed by labeled cells, (xvi) a therapeutic agent followed by a vascular permeability agent followed by labeled cells, (xvii) a therapeutic agent followed by labeled cells followed by a vascular permeability agent, (xviii) labeled cells followed by a therapeutic agent followed by a vascular permeability agent, (xix) labeled cells followed by a vascular permeability agent followed by a therapeutic agent, (xx) a vascular permeability agent followed by labeled cells and a therapeutic agent, (xxi) a therapeutic agent followed by a vascular permeability agent and labeled cells, (xxii) labeled cells followed by a therapeutic agent and vascular agent, or any combination thereof.

The vascular agents permeability provided herein can be administered to a target tissue or organ by various known methods known in the art, such as by injection (e.g., direct needle injection at the delivery site, subcutaneously or intravenously), oral administration, inhalation, transdermal application, catheter infusion, biolistic injectors, particle accelerators, GELFOAM®, other commercially available depot materials, osmotic pumps, oral or suppositorial solid pharmaceutical formulations, decanting or topical applications during surgery, or aerosol delivery. Depending on the route of administration, the composition can be coated with a material to protect the agents from the action of acids and other natural conditions which can inactivate the agents. In some embodiments, the agents are locally administered to the target tissue or organ (e.g., cardiac tissue).

In some embodiments, the agents are perfused or otherwise gently administered near or directly to the pre-determined region of interest. Choice of a particular perfusion rate will be guided by the particular tissue or organ for which treatment is desired. For example, a perfusion rate through a tissue or organ region of interest can be between about 0.5 mL/min to about 500 mL/min. In some embodiments where cardiac tissue is to be treated, the perfusion rates can be between about 1 mL/min to about 100 mL/min, including between about 10 mL/min to about 20 mL/min, 20 mL/min to about 30 mL/min, 30 mL/min to about 40 mL/min, 40 mL/min to about 50 mL/min, 50 mL/min to about 60 mL/min, 60 mL/min to about 70 mL/min, 70 mL/min to about 80 mL/min, 80 mL/min to about 90 mL/min, 90 mL/min to about 100 mL/min, and overlapping ranges thereof.

The amount of vascular permeability agent to be used in the methods provided herein will depend on recognized factors such as the agent selected, the perfusion rate desired, etc. In some embodiments, the agent is used in an amount of about 0.01 μmoles/L to about 500 μmoles/L. In some embodiments, the agent is used in an amount ranging from about 0.01 μmoles/L to about 0.1 μmoles/L, about 0.1 μmoles/L to about 1 μmoles/L, about 1 μmoles/L to about 10 μmoles/L, about 10 μmoles/L to about 50 μmoles/L, about 50 to about 100 μmoles/L, about 100 to about 200 μmoles/L, about 200 to about 300 timoles/L, about 300 to about 400 timoles/L, about 400 to about 500 μmoles/L, and overlapping ranges thereof. In still additional embodiments, combinations of one or more vascular permeability agents are used. In some embodiments, the total concentration is within the ranges discussed above, while in some embodiments, the combined concentration is with the ranges discussed above. In some embodiments, the agent(s) is combined with a suitable pharmaceutically acceptable vehicle prior to being administered to a subject.

One or a combination of selected vascular permeability agents can be administered to the subject for nearly any length of time needed to provide for good contact of agent(s) with the tissue or organ region of interest. In one embodiment, the vascular permeability agent is contacted with (e.g., perfused through) the region for between about 10 seconds to about five or six hours, about 10 minutes to about two hours, or about 30 minutes to about an hour. Choice of a particular perfusion time will depend on the particular tissue or organ region for which treatment is desired fu some embodiments in which cardiac tissue is to be treated, the time for perfusion can be less than about two hours. In one embodiment in which cardiac tissue is to be treated, the time for perfusion is between from about 30 seconds to about an hour. In other embodiments times range from about 5 to about 10 seconds, about 10 to about 30 seconds, about 30 to about 60 seconds, about 1 minute to about 5 minutes, about 5 minutes to about 10 minutes, about 10 minutes to about 15 minutes, about 15 minutes to about 30 minutes, about 30 minutes to about 60 minutes, and overlapping ranges thereof.

Non-limiting examples of vascular permeability agents suitable for use in the compositions and methods provided herein include the following: substance P, histamine, acetylcholine, an adenosine nucleotide, arachidonic acid, bradykinin, endothelin, endotoxin, interleukin-2, nitric oxide agonists or promoters (activators) such as nitroglycerin and nitroprusside, nitric oxide, a leukotriene, an oxygen radical, phospholipase, platelet-activating factor (PAF), protamine, serotonin, tumor necrosis factor, a venom, a vasoactive amine, nitric oxide synthase inhibitor, prostaglandin E (e.g., prostaglandin E1), histamine, zona occludens toxin (ZOT), plasma kinins, L-N-monomethyl arginine, L-N-nitro-arginine methyl ester, and 8-BrcGMP, recombinant adenoviruses (e.g., use of recombinant adenovirus in the heart), agents that can increase cyclic guanonisine 3′-monoposphate (cGMP), and vascular endothelial growth factor (VEGF) or a functional fragment or a derivative thereof (e.g., VEGF 165), or any combination thereof. In some embodiments, the VEGF derivative is VEGF165. Various vascular permeability agents including VEGF are available from a variety of commercial sources such as Sigma-Aldrich (St. Louis, Mo.). For more information regarding VEGF derivatives, see, e.g., U.S. Pat. Nos. 6,020,473 and 6,057,428, herein incorporated by reference.

Other vascular permeability agents suitable for use in the compositions and methods provided herein include various vasodilators. Examples of vasodilators include but are not limited to: angiotensin converting enzyme (ACE) inhibitor, angiotensin II receptor antagonist, a nitrovasodilator, phosphodiesterase (PDE) inhibitor (e.g., PDE-5 inhibitor), direct vasodilator, adrenergic receptor antagonist, calcium channel blocking agent, or a sympathomimetic. In some embodiments, the vasodilator is nitroglycerin. PDE inhibitors suitable for use in the compositions and methods provided herein include, but are not limited to, bicyclic heterocylic PDE inhibitors, sildenafil, zaprinast, T-1032 (Tanabe Seiyaku Co.), pyrazolo[4,3-d]prymidin-7-ones, pryazolo[3,4-d]pyrimidin-4-ones, quinazolin-4-ones, purin-6-ones, pyrido[3,2-d]pyrimidin-4-ones, and pharmaceutically acceptable salts thereof. In a one embodiment, the PDE inhibitor suitable for use in the compositions and methods provided herein is pyrazolo[4,3-d]prymidin-7-one, also known as sildenafil (Viagra™) and 5-[2-ethoxy-5-(4-methylpiperazin-1-yl sulphonyl)phenyl]-1-methyl-3-n-propyl-1,6-dihydro-7H-pyrazolo[4,3d]pyrimidin-7-one, and pharmaceutically acceptable salts thereof, or any combination thereof. For more information regarding PDE inhibitors, see, e.g., U.S. Pat. No. 6,992,070, herein incorporated by reference.

Yet other vascular permeability agents suitable for use in the compositions and methods provided herein include a “low calcium” solution. Such solutions, when used as a vascular permeability agent, can have less than about 500 μmoles/L of a calcium salt or less than 100 μmoles/L of the salt, with between from about 1 μM to about 50 μM being useful for many applications. Suitable calcium salts for use in the compositions and methods provided include chloride salts and other salts, such as inorganic and organic acid addition salts of calcium (e.g., sulphates, nitrates or phosphates and acetates, trifluoroacetates, propionates, succinates, benzoates, citrates, tartrates, fumarates, maleates, methanesulfonates, isothionates, theophylline acetates, salicylates) and lower alkyl quaternary ammonium salts. Pharmaceutically acceptable anions include, but are not limited to, CH₃COO⁻, CF₃COO⁻, CI⁻, SO₃²⁻, maleate and oleate. In some embodiments, a combination of vascular permeability agents includes use of at least two of serotonin, VEGF or a derivative thereof (e.g., VEGF 165), and nitroglycerin. The combination may be used alone or in combination with a low calcium solution to assist activity of the recombinant animal virus. More information regarding suitable vascular permeability agents for use with the compositions and methods provided herein can be found, e.g., in U.S. Pat. Nos. 6,992,070 and 7,034,008, U.S. Pat. Publ. Nos. 2004/0204376, 2002/0094326 and 2002/0155101, and Neyroud, et al. (2002) Methods In Enzymol. 346:323.

Any combination of one or more vascular permeability agents can be used in the methods, compositions and kits provided herein. Such vascular permeability agents can also be used in combination with anyone or more of the therapeutic agents and/or labeled cells provided herein.

The vascular permeability agents suitable for use in the compositions and methods provided herein will often be combined with one or more physiologically acceptable carriers such as sterile water, sterile saline, e.g., isotonic saline. Other physiologically acceptable carriers are known in the field and may be used in some embodiments disclosed herein.

An effective dose of one or more vascular permeability agent that is administered with magnetic particle-labeled cells to a target tissue or organ will vary depending on the cell type used, the delivery site (e.g., intracoronary or intramyocardial), and the patient (e.g., weight) and such doses can be readily determined by a physician (see also, e.g., Physician's Desk Reference, 63rd Ed. (2009) Thomson PDR (Montvale, N.J.); herein incorporated by reference). Patient age, general condition, and immunological status may be used as factors in determining the dose administered, and will be readily determined by the physician.

For example, in some embodiments, the methods provided herein involve contacting cardiac tissue with magnetic particle-labeled cells and/or one or more vascular permeability agents. The vascular permeability agents can be administered to a cardiac tissue by various means known in the art. For example, in some embodiments, one or more of the vascular permeability agents, either alone or in combination, and optionally in combination with the labeled cells (e.g., labeled stem cells such as CDCs) are administered to a cardiac tissue via intracoronary infusion. In some embodiments, one or more of the vascular permeability agents, either alone or in combination, and optionally in combination with the labeled cells, are directly injected epicardially into a cardiac tissue, for example, during an open chest surgery. In other embodiments, one or more of the vascular permeability agents, either alone or in combination, and optionally in combination with the labeled cells are administered to the cardiac tissue using non-surgical methods, for example, by intravascular (e.g., intracoronary or intravenous) or intramyocardial administration. One or more of the vascular permeability agents, either alone or in combination, and optionally in combination with the labeled cells, that are administered to cardiac tissue using non-surgical methods can be prepared, for example, in an injectable liquid suspension or any other biocompatible medium. For intravascular approaches, catheters may be advanced through the vasculature and into the heart to inject one or more of the vascular permeability agents, either alone or in combination, and optionally in combination with the labeled cells, into the cardiac tissue from within the heart. In one embodiment, one or more of the vascular permeability agents, either alone or in combination, and optionally in combination with the labeled cells, are administered to the cardiac tissue by intracoronary administration. In another embodiment, one or more of the vascular permeability agents, either alone or in combination, and optionally in combination with the labeled cells, are administered to the cardiac tissue, for example, by intravenous administration, by continuous drip or as a bolus. In yet another embodiment, one or more of the vascular permeability agents, either alone or in combination, and optionally in combination with the labeled cells, are contacted with the cardiac tissue by intramyocardial administration, for example, using a conventional intracardiac syringe or a controllable endoscopic delivery device. In some embodiments, one or more of the vascular permeability agents, either alone or in combination, and optionally in combination with the labeled cells, are administered to the cardiac tissue using an endocardial approach that delivers the agent(s) and/or labeled cells into the cardiac wall from within the chamber of the heart.

In some embodiments, a vascular permeability agent is administered to the cardiac tissue or organ prior to administration of the labeled cells. In some embodiments, a vascular permeability agent is administered between about 1 minute and about 60 minutes prior to administration of the labeled cells. In some embodiments, a vascular permeability agent is administered to, for example, a cardiac tissue within 1, 5, 10, 15, 20, 30, 45 minutes or about 1, 2, 4, 6, 10, 12, 18, 20 or 24 hours of administration of the labeled cells. In one embodiment, the labeled cells are subsequently administered to the cardiac tissue, e.g., within 1, 5, 10, 15, 20, 30, 45 minutes or about 1, 2, 4, 6, 10, 12, 18, 20 or 24 hours of administration of the vascular permeability agent. In yet other embodiments, a vascular permeability agent is administered concurrently with the labeled cells. In some embodiments, the labeled cells are administered to a cardiac tissue prior to administration of the a vascular permeability agent.

In some embodiments of the methods provided herein, the vascular permeability agent is administered to the peri-infarct zone of a cardiac tissue. In some embodiments of the methods provided herein, the vascular permeability agents are administered into the peri-infarct zone concurrently or sequentially (i.e., before or after) with labeled cells (e.g., CDCs).

In some embodiments, one or more of the vascular permeability agents, either alone or in combination with each other, are administered in one or more systems, e.g., a long-term, short-term and/or controlled release system(s) that optionally further comprise the labeled cells. In one embodiment, the labeled cells are provided in a release system with one or more of the vascular permeability agents. In another embodiment, the labeled cells are provided in a release system, but none of the vascular permeability agents are provided in a release system. In other embodiments, one or more vascular permeability agents are provided in one or more releases systems (the same or different), but the labeled cells are not provided in a release system. In some embodiments, the system is a matrix, such as a natural or synthetic matrix (see, e.g., Simpson et al. (2007) Stem Cells 25:2350; herein incorporated by reference).

In some embodiments, one or more of the vascular permeability agents, either alone or in combination with each other, and optionally in combination with the labeled cells, are administered in a biocompatible medium which is, or becomes a semi-solid or solid matrix in situ at the site of myocardial damage, such as any of the matrixes described herein. In some embodiments, one or more of the vascular permeability agents, either alone or in combination with each other, and optionally in combination with the labeled cells, are embedded into a tissue-engineered cardiac patch containing, for example, a collagen matrix. Such a patch can then be attached or otherwise delivered to the cardiac tissue, for example, with a sealant (e.g., fibrin) (see, e.g., Simpson et al. (2007) Stem Cells 25:2350; herein incorporated by reference).

In some embodiments, one or more of the vascular permeability agents, either alone or in combination with each other, and optionally in combination with the labeled cells, are administered to the cardiac tissue once, either concurrently (e.g., vascular permeability agents and labeled cells) or sequentially (e.g., vascular permeability agents then labeled cells, labeled cells then vascular permeability agents, or vascular permeability agent then labeled cells, then vascular permeability agents, for example, within minutes or hours). In other embodiments, one or more of the vascular permeability agents, either alone or in combination with each other, and optionally in combination with the labeled cells, are concurrently or sequentially administered to cardiac tissue more than one time (e.g., several hours, days or months apart).

In some embodiments of the methods provided herein, one or more of the vascular permeability agents, either alone or in combination with each other, and optionally in combination with the labeled cells, are administered to the cardiac tissue of the patient after tissue injury occurs but before or coincident with reperfusion (e.g., after vascular occlusion but before or coincident with angioplasty).

In some embodiments, one or more of the vascular permeability agents, either alone or in combination with each other, and optionally in combination with the labeled cells, are administered in a pharmaceutically acceptable liquid medium (e.g., saline or buffer), for example, for systemic administration or local administration, e.g., directly into the damaged portion of the myocardium. In some embodiments, administration is localized to the cardiac tissue.

One or more of the methods of delivery or formulations provided herein can be used to contact the cardiac tissue with one or more of the vascular permeability agents, either alone or in combination with each other, and the labeled cells. For example, in some embodiments, one or more of the vascular permeability agents are contacted with the cardiac tissue by a first method of delivery and/or in a first formulation (e.g., direct needle injection of liquid formulation), and the labeled cells are concurrently or sequentially contacted with the cardiac tissue by a second method of delivery and/or in a second formulation (e.g., matrix).

Therapeutic Agents for Use with Magnetic Particle-Labeled Cells

In some embodiments, the labeled cells provided herein can optionally be utilized in conjunction with one or more therapeutic drugs or agents, either magnetized or unmagnetized, and/or genes expressing the same. Such therapeutic drugs or agents include, for example, antineoplastic agents, anti-angiogenic or pro-angiogenic factors, immuno-suppressants, or antiproliferatives (anti-restenosis agents). Other non-limiting examples include embryonic factors, a fibroblast growth factors, transcription factors, kinase inhibitors, or adenosine. The therapeutic drugs or agents can be contacted with the cardiac tissue by any of a variety of procedures known in the art either alone or in combination with each other, and optionally in combination with the magnetized cells. If metalloliposomes are used to label the cells, for example, the therapeutic agents can also be encapsulated in the liposome (see, e.g., Lubbe et al. (2001) J. Surg. Res. 95:200-206; herein incorporated by reference).

Therapeutic agents that can be used in combination with the labeled cells in the compositions, methods or kits provided herein include (e.g., one, two, three, four or more) drug(s) or other agent(s). Such a drug can be anyone or more of an anti-neoplastic drug, anti-angiogenesis drug, pro-angiogenesis drug, anti-fungal drug, anti-viral drug, anti-inflammatory drug, anti-bacterial drug, a cytotoxic drug, a chemotherapeutic or pain relieving drug and/or an anti-histamine drug. The drug can also be, for example, anyone or more of hormones, steroids, vitamins, cytokines, chemokines, growth factors, interleukins, enzymes, anti-allergenic agents, circulatory drugs, anti-tubercular agents, anti-anginal agents, anti-protozoan agents, anti-rheumatic agents, narcotics, cardiac glycoside agents, sedatives, local anesthetic agents, general anesthetic agents, and combinations thereof. In some embodiments, the therapeutic agent is an anti-neoplastic, chemotherapeutic or pain relieving drug.

Examples of anti-angiogenic or anti-neoplastic drugs include, without limitation, alkylating agents, nitrogen mustards, antimetabolites, gonadotropin releasing hormone antagonists, androgens, antiandrogens, antiestrogens, estrogens, and combinations thereof. Examples include but are not limited to actinomycin D, aldesleukin, alemtuzumab, alitretinoin, allopurinol, altretamine, amifostine, aminoglutehimide, amphotercin B, amsacrine, anastrozole, ansamitocin, arabinosyl adenine, arsenic trioxide, asparaginase, aspariginase Erwinia, BCG Live, benzamide, bevacizumab, bexarotene, bleomycin, 3-bromopyruvate, busulfan, calusterone, capecitabine, carboplatin, carzelesin, carmustine, celecoxib, chlorambucil, cisplatin, cladribine, cyclophosphamide, cytarabine, cytosine arabinoside, dacarbazine, dactinomycin, darbepoetin alfa, daunorubicin, daunomycin, denileukin diftitox, dexrazoxane, dexamethosone, docetaxel, doxorubicin, dromostanolone, epirubicin, epoetin alfa, estramustine, estramustine, etoposide, VP-16, exemestane, filgrastim, floxuridine, fludarabine, fluorouracil (5-FU), flutamide, fulvestrant, demcitabine, gemcitabine, gemtuzumab, goserelin acetate, hydroxyurea, ibritumomab, idarubicin, ifosfamide, imatinib, interferon (e.g., interferon α-2a, interferon α-2b), irinotecan, letrozole, leucovorin, leuprolide, lomustine, meciorthamine, megestrol, melphalan (e.g., PAM, L-PAM or phenylalanine mustard), mercaptopurine, mercaptopolylysine, mesna, mesylate, methotrexate, methoxsalen, mithramycin, mitomycin, mitotane, mitoxantrone, nandrolone phenpropionate, nolvadex, oprelvekin, oxaliplatin, paclitaxel, pamidronate sodium, pegademase, pegaspargase, pegfilgrastim, pentostatin, pipobroman, plicamycin, porfimer sodium, procarbazine, quinacrine, raltitrexed, rasburicase, riboside, rituximab, sargramostim, spiroplatin, streptozocin, tamoxifen, tegafur-uracil, temozolomide, teniposide, testolactone, tioguanine, thiotepa, tissue plasminogen activator, topotecan, toremifene, tositumomab, trastuzumab, treosulfan, tretinoin, trilostane valrubicir, vinblastine, vincristine, vindesine, vinorelbine, zoledronate, salts thereof, or mixtures thereof. In some embodiments, the platinum compound is spiroplatin, cisplatin, or carboplatin. In some embodiments, the drug is cisplatin, mitomycin, paclitaxel, tamoxifen, doxorubicin, tamoxifen, or mixtures thereof.

Other anti-angiogenic or anti-neoplastic drugs include, but are not limited to AGM-1470 (TNP-470), angiostatic steroids, angiostatin, antibodies against avβ3, antibodies against bFGF, antibodies against IL-1, antibodies against TNF-α, antibodies against VEGF, auranofin, azathioprine, BB-94 and BB-2516, basic FOF-soluble receptor, carboxyamido-trizole (CAI), cartilage-derived inhibitor (CDI), chitin, chloroquine, CM 101, cortisonelheparin, cortisonelhyaluroflan, cortexolonelheparin, CT-2584, cyclophosphamide, cyclosporin A, dexamethasone, diclofenaclhyaluronan, eosinophilic major basic protein, fibronectin peptides, Glioma-derived angiogenesis inhibitory factor (GD-AIF), GM 1474, gold chloride, gold thiomalate, heparinases, hyaluronan (high and low molecular-weight species), hydrocortisonelbeta-cyclodextran, ibuprofen, indomethacin, interferon-α, interferon 7-inducible protein 10, interferon-γ, IL-1, IL-2, IL-4, IL-12, laminin, levamisole, linomide, LM609, martmastat (BB-2516), medroxyprogesterone, methotrexate, minocycline, nitric oxide, octreotide (somatostatin analogue), D-penicillamine, pentosan polysulfate, placental proliferin-related protein, placental RNase inhibitor, plasminogen activator inhibitor (PAIs), platelet factor-4 (PF4), prednisolone, prolactin (16-kDa fragment), proliferin-related protein, prostaglandin synthase inhibitor, protamine, retinoids, somatostatin, substance P, suramin, SU101, tecogalan sodium (05-4152), tetrahydrocortisol-sthrombospondins (TSPs), tissue inhibitor of metalloproteinases (TIMP 1,2,3), thalidomide, 3-aminothalidomide, 3-hydroxythalidomide, metabolites or hydrolysis products of thalidomide, 3-aminothalidomide, 3-hydroxythalidomide, vitamin A and vitreous fluids. In another embodiment, the anti-angiogenic agent is selected from the group consisting of thalidomide, 3-aminothalidomide, 3-hydroxythalidomide and metabolites or hydrolysis products of thalidomide, 3-aminothalidomide, 3-hydroxy thalidomide. In one embodiment, the anti-angiogenic agent is thalidomide.

Examples of pain reliving drugs are, without limitation, analgesics or anti-inflammatories, such as non-steriodal anti-inflammatory drugs (NSAID), ibuprofen, ketoprofen, dexketoprofen, phenyltoloxamine, chlorpheniramine, furbiprofen, vioxx, celebrex, bexxstar, nabumetone, aspirin, codeine, codeine phosphate, acetaminophen, paracetamol, xylocalne, and naproxin. In some embodiments, the pain relieving drug is an opioid. Opioids are commonly prescribed because of their effective analgesic, or pain relieving, properties. Among the compounds that fall within this class include narcotics, such as morphine, codeine, and related medications. Other examples of opioids include oxycodone, propoxyphene, hydrocodone, hydromorphone, and meperidine. Narcotics, include, for example, without limitation, paregoric and opiates, such as codeine, heroin, methadone, morphine and opium.

Hormones and steroids, include, for example, without limitation, growth hormone, melanocyte stimulating hormone, adrenocortiotropic hormone, dexamethasone, dexamethasone acetate, dexamethasone sodium phosphate, cortisone, cortisone acetate, hydrocortisone, hydrocortisone acetate, hydrocortisone cypionate, hydrocortisone sodium phosphate, hydrocortisone sodium succinate, prednisone, prednisolone, prednisolone acetate, prednisolone sodium phosphate, prednisolone tebutate, prednisolone pivalate, triamcinolone, triamcinolone acetonide, triamcinolonehexacetonide, triamcinolone acetate, methylprednisolone, methylprednisolone acetate, methylprednisolone sodium succinate, flunsolide, beclomethasone dipropionate, betamethasone sodium phosphate, betamethasone, vetamethasone disodium phosphate, vetamethasone sodium phosphate, betamethasone acetate, betamethasone disodium phosphate, chloroprednisone acetate, corticosterone, desoxycorticosterone, desoxycorticosterone acetate, desoxycorticosterone pivalate, desoximethasone, estradiol, fludrocortisone, fludrocortisoneacetate, dichlorisone acetate, fluorohydrocortisone, fluorometholone, fluprednisolone, paramethasone, paramethasone acetate, androsterone, fluoxymesterone, aldosterone, methandrostenolone, methylandrostenediol, methyl testosterone, norethandrolone, testosterone, testosteroneenanthate, testosterone propionate, equilenin, equilin, estradiol benzoate, estradiol dipropionate, estriol, estrone, estrone benzoate, acetoxypregnenolone, anagestone acetate, chlormadinone acetate, fluorogestone acetate, hydroxymethylprogesterone, hydroxymethylprogesterone acetate, hydroxyprogesterone, hydroxyprogesterone acetate, hydroxyprogesterone caproate, melengestrol acetate, normethisterone, pregnenolone, progesterone, ethynyl estradiol, mestranol, dimethisterone, ethisterone, ethynodiol diacetate, norethindrone, norethindrone acetate, norethisterone, fluocinolone acetonide, flurandrenolone, flunisolide, hydrocortisone sodium succinate, methylprednisolone sodium succinate, prednisolone phosphate sodium, triamcinolone acetonide, hydroxydione sodium spironolactone, oxandrolone, oxymetholone, prometholone, testosterone cypionate, testosterone phenyl acetate, estradiol cypionate, and norethynodrel.

Peptides and peptide analogs, include, for example, without limitation, manganese super oxide dismutase, tissue plasminogen activator (t-PA), glutathione, insulin, dopamine, peptide ligands containing RGD, AGD, RGE, KGD, KGE or KQAGDV (peptides with affinity for theGPEXma receptor), opiate peptides, enkephalins, endorphins and their analogs, human chorionic gonadotropin (HCG), corticotropin release factor (CRF), cholecystokinins and their analogs, bradykinins and their analogs and promoters and inhibitors, elastins, vasopressins, pepsins, glucagon, substance P, integrins, captopril, enalapril, lisinopril and other ACE inhibitors, adrenocorticotropic hormone (ACTH), oxytocin, calcitonins, IgG or fragments thereof, IgA or fragments thereof, IgM or fragments thereof, ligands for Effector Cell Protease Receptors (all subtypes), thrombin, streptokinase, urokinase, t-PA and all active fragments or analogs, Protein Kinase C and its binding ligands, interferons (α-IFN, β-IFN, γ-IFN), colony stimulating factors (CSF), granulocyte colony stimulating factors (GCSF), granulocytemacrophage colony stimulating factors (GM-CSF), tumor necrosis factors (TNF), nerve growth factors (NGF), platelet derived growth factors, lymphotoxin, epidermal growth factors, fibroblast growth factors, vascular endothelial cell growth factors, erythropoietin, transforming growth factors, oncostatin M, interleukins (IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, etc.), metalloprotein kinase ligands, collagenases and agonists and antagonists.

Antibodies, include, for example, without limitation, substantially purified antibodies or fragments thereof, including non-human antibodies or fragments thereof. In various embodiments, the substantially purified antibodies or fragments thereof, can be human, nonhuman, chimeric and/or humanized antibodies. Such non-human antibodies can be goat, mouse, sheep, horse, chicken, rabbit, or rat antibodies. The antibodies can be monoclonal or polyclonal antibodies.

Anti-mitotic factors include, without limitation, estramustine and its phosphorylated derivative, estramustine-phosphate, doxorubicin, amphethinile, combretastatin A4, and colchicine.

Anti-coagulation agents, include, for example, without limitation, phenprocoumon and heparin.

Circulatory drugs, include, for example, without limitation, propranolol.

Anti-viral agents, include, for example, without limitation, acyclovir, amantadine azidothymidine (AZT or Zidovudine), ribavirin, and vidarabine monohydrate (adenine arabinoside,ara-A).

Anti-anginal agents, include, for example, without limitation, diltiazem, nifedipine, verapamil, erythritol tetranitrate, isosorbide dinitrate, nitroglycerin (glyceryl trinitrate), and pentaerythritolteiranitrate.

Antibiotics, include, for example, dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin G, penicillin V, ticarcillin, rifampin, and tetracycline.

Anti-inflammatory agents and analgesics, include, for example, diflunisal, ibuprofen, indomethacin, meclofenamate, mefenamic acid, naproxen, oxyphenbutazone, phenylbutazone, piroxicam, sulindac, tolmetin, aspirin and salicylates.

Cardiac glycoside agents, include, for example, without limitation, deslanoside, digitoxin, digoxin, digitalin and digitalis.

Neuromuscular blocking agents, include, for example, without limitation, atracurium mesylate, gallamine triethiodide, hexafluorenium bromide, metocurine iodide, pancuronium bromide, succinylcholine chloride(suxamethonium chloride), tubocurarine chloride, and vecuronium bromide.

Sedatives, include, for example, without limitation, amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride paraldehyde, pentobarbital, pentobarbital sodium, phenobarbital sodium, secobarbital sodium, talbutal, temazepam, and triazolam.

Local anesthetic agents, include, for example, without limitation, bupivacaine hydrochloride, chloroprocaine hydrochloride, etidocaine hydrochloride, lidocaine hydrochloride, mepivacaine hydrochloride, procaine hydrochloride, and tetracaine hydrochloride.

General anesthetic agents, include, for example, without limitation, droperidol, etomidate, fentanyl citrate with droperidol, ketamine hydrochloride, methohexital sodium, and thiopental sodium.

Radioactive particles or ions, include, for example, without limitation, strontium, rhenium, yttrium, technetium, and cobalt.

Any combination of one or more therapeutic agents or dugs can be used in the methods, compositions and kits provided herein. Such therapeutic agents or drugs can also be used in combination with anyone or more of the vascular permeability agents and/or labeled cells provided herein.

In some embodiments of the compositions, methods and kits provided herein, the therapeutic agent is also a vascular permeability agent.

### Compositions

Compositions for carrying out the methods described herein are also provided herein. In some embodiments, stem cells are used. In some embodiments, other cells are used (e.g., hepatocytes, fibroblasts, etc.) For example, provided herein is a composition, comprising magnetically labeled CDCs. In some embodiments, the composition further comprises a vascular permeability agent and/or a therapeutic agent or drug. In another embodiment, provided herein is a composition, comprising: magnetically labeled cells and a vascular permeability agent, wherein the composition optionally further comprises a therapeutic agent or drug.

### Kits

Also provided herein is a pharmaceutical pack or kit that can be used in a method provided herein, wherein said pharmaceutical pack, bag, or kit comprises one or more containers (e.g., vials or syringes) filled with one or more of the ingredients of the compositions provided herein, such as magnetically labeled cells (e.g., CDCs). Optionally associated with such container(s) can be a notice in the form prescribed by a governmental agency regulating the manufacture, use or sale of pharmaceuticals or biological products, which notice reflects approval by the agency of manufacture, use or sale for human administration (e.g., instructions for use). In one embodiment, a kit comprises magnetically labeled CDCs, and optionally further comprises a vascular permeability agent and/or a therapeutic agent or drug. In another embodiment, the kit comprises magnetically labeled cells and a vascular permeability agent, and optionally further comprises a therapeutic agent or drug. In some embodiments, the kits provided herein further comprise a magnet.

In several embodiments, methods are provided for magnetically targeting cells into a heart to repair damaged cardiac tissue, comprising delivering magnetically-labeled cardiac stem cells to one or more delivery sites of a heart comprising a region of damaged cardiac tissue, wherein the cardiac tissue is damaged as a result of injury or disease, wherein the damaged cardiac tissue has reduced cardiac function, wherein the delivery is performed while the heart is actively contracting, wherein the active contraction induces an efflux of the delivered cells away from the delivery site, and transiently applying a magnetic field around or adjacent to the damaged cardiac tissue, wherein the magnetic field counteracts the efflux of the delivered cells and enhances the short-term retention and long-term engraftment of the delivered cells, wherein the enhanced retention and engraftment provide long-term functional and anatomical improvements in the region of damaged cardiac tissue, thereby repairing the damaged cardiac tissue. In one embodiment, the cardiac stem cells comprise cardiosphere derived cells. In one embodiment, the magnetic label comprises super paramagnetic iron oxide (SPIO) particles. In one embodiment, the SPIO particles comprise superparamagnetic microsphere s (SPM). In one embodiment, the ratio of label to cell is about 500:1. In one embodiment, the damaged cardiac tissue results from an acute injury to the heart, such as a myocardial infarction. In one embodiment, the damaged cardiac tissue results from chronic stress or disease of the heart, such as one or more of chronic heart failure, systemic hypertension, pulmonary hypertension, valve dysfunction, congestive heart failure, and coronary artery disease. In one embodiment, the damaged cardiac tissue is epicardium, endocardium, or myocardium. In one embodiment, the functional improvement comprises an increase in cardiac output. In one embodiment, the increase in cardiac output comprises an increase in left ventricular ejection fraction, which is increased by least 5% or by about 10%. In one embodiment, the repair of the damaged cardiac tissue also results in an increase in viable cardiac tissue. In one embodiment, the anatomical improvements comprise an increase in cardiac wall thickness. In one embodiment, the damaged cardiac tissue is a result of a myocardial infarction and wherein the repair of the damaged cardiac tissue further comprises a decrease in scar tissue formation. In one embodiment, the delivered cells engraft into the damaged cardiac tissue as focal patches of cells. In one embodiment, the magnetic label in or on the magnetically-labeled stem cells is reduced over time after delivery. In one embodiment, the short term retention is enhanced by at least 10% as compared to non-magnetically targeted cells. In one embodiment, long term engraftment is enhanced by at least 10% as compared to non-magnetically targeted cells. In one embodiment, the magnetic field is applied via one or more magnetic sources positioned external to the heart. In one embodiment, the magnetic field is applied via a catheter having a magnetic tip. In one embodiment, the magnetic field has a field strength of about 0.1 Tesla to about 100 Tesla, including about 1.3 Tesla. In one embodiment, the cardiac stem cells are autologous cardiosphere-derived cells while in one embodiment the cardiac stem cells are allogeneic cardiosphere-derived cells. In one embodiment, the delivery of the magnetically-labeled stem cells does not preferentially attract macrophages to the region of damaged cardiac tissue. In one embodiment, the repair of the damaged cardiac tissue is due to proliferation of the delivered cells within or adjacent to the region of damaged cardiac tissue. In one embodiment, the repair of the damaged cardiac tissue is due to paracrine modulators released from the delivered cells, wherein the paracrine modulators improve the viability of cardiac tissue and/or recruit endogenous cardiac cells to the region of damaged cardiac tissue.

In several embodiments, there is provided a method for the repair or regeneration of damaged cardiac tissue, comprising delivering magnetically-labeled stem cells to a subject having a heart comprising a region of damaged cardiac tissue with compromised cardiac function, wherein the stem cells are cardiac stem cells, wherein the magnetically-labeled stem cells are delivered within or adjacent to the region of damaged cardiac tissue and applying a magnetic field around or adjacent to the damaged cardiac tissue, wherein the magnetic field enhances one or more of the delivery, short term retention, or long-term engraftment of the magnetically-labeled stem cells within or adjacent to the region of damaged cardiac tissue, wherein the enhanced delivery, retention, or engraftment results in functional improvement in the region of damaged cardiac tissue. In one embodiment, the functional improvement comprises an increase in left ventricular ejection fraction. In one embodiment, the repair of damaged cardiac tissue also yields an increase in viable cardiac tissue and/or cardiac wall thickness.

In several embodiments, there is provided a method for the improving the function of cardiac tissue damaged as a result of a myocardial infarction, comprising delivering magnetically-labeled cardiac stem cells to a subject having been afflicted with a myocardial infarction, wherein the magnetically-labeled stem cells are delivered via a catheter have an electromagnetic portion, and generating a magnetic field from the catheter, wherein the magnetic field enhances the retention of the magnetically-labeled cardiac stem cells at region of damaged cardiac tissue, wherein the enhanced retention results in enhanced engraftment of the magnetically-labeled cardiac stem cells, wherein the enhanced retention and engraftment produce healthy myocardium at the region of damaged cardiac tissue, and wherein the healthy myocardium results in functional improvement of the damaged cardiac tissue.

In several embodiments, there is provided the use of magnetically-labeled cardiac stem cells for the repair of damaged cardiac tissue, wherein the magnetically-labeled cardiac stem cells are cardiosphere-derived cells labeled with SPM particles, wherein the magnetically-labeled cardiac stem cells are suitable for delivery to the heart of a subject having damaged cardiac tissue, wherein application of magnetic field applied around the damaged cardiac tissue results in increased retention of delivered magnetically-labeled cardiac stem cells, and wherein increased retention of delivered magnetically-labeled cardiac stem cells leads to functional improvement of the damaged cardiac tissue, thereby repairing the damaged cardiac tissue.

The following examples are provided to further illustrate certain embodiments within the scope of the invention. The examples are not to be construed as a limitation of any embodiments, since numerous modifications and variations are possible without departing from the spirit and scope of the invention.

### EXAMPLES

### Example 1

### Isolation of Cardiac-Derived Stem Cells from Cardiac Biopsy Specimens

Pluripotent stem cells can be isolated from cardiac biopsy specimens or other cardiac tissue using any known methods, for example, the multi-step process described in U.S. Publication No. 2008/026792, which is incorporated herein by reference in its entirety.

Utilizing such method, cardiac tissue was first obtained via percutaneous endomyocardial biopsy or via sterile dissection of the heart. It shall be appreciated, however, that in other embodiments, the original tissue may be obtained by other means (e.g., surgical specimens, fresh cadaveric tissue, etc.). In some embodiments, fresh tissue harvesting is unnecessary, as stem cells that have previously been isolated and banked (e.g., stored frozen) are used. Once obtained, tissue specimens were stored on ice in a high-potassium cardioplegic solution (containing 5% dextrose, 68.6 mmol/L mannitol, 12.5 meq potassium chloride, and 12.5 meq sodium bicarbonate, with the addition of 10 units/mL of heparin) until they processing. Processing, in some embodiments, may be performed up to about 12 hours later). For processing, specimens were cut into 1-2 mm³ pieces using sterile forceps and scissors; any gross connective tissue was removed. The fragments were then washed with Ca⁺⁺/Mg⁺⁺-free phosphate buffered saline (PBS) and digested for about 5 min at room temperature with 0.05% trypsin-EDTA. Alternatively the tissue fragments may be digested in type IV collagenase (1 mg/mL) for about 30 minutes at 37° C. Depending on the individual specimen, digestion of the fragment may be performed for longer or shorter periods of time. Preliminary experiments have shown that cellular yield is greater per mg of explant tissue when collagenase is used.

Once digestion was complete, the remaining tissue fragments were washed with “Complete Explant Medium” (CEM) containing 20% heat-inactivated fetal calf serum, 100 Units/mL penicillin G, 100 μg/mL streptomycin, 2 mmol/L L-glutamine, and 0.1 mmol/L 2-mercaptoethanol in Iscove's modified Dulbecco medium to quench the digestion process. The tissue fragments were minced again with sterile forceps and scissors and then transferred to fibronectin-coated (25 μg/mL for at least 1 hour) tissue culture plates. The fragments were placed, evenly spaced, across the surface of the plate. A minimal amount of CEM was added to the plate, after which it was incubated at 37° C. in 5% CO₂ for 30 minutes to allow the tissue fragments, now referred to as “explants”, to attach to the plate. Once the explants were attached, enough CEM was added to the plate to cover the explants, and the plates were returned to the incubator.

After a period of 8 or more days, a layer of stromal-like cells arose from adherent explants, covering the surface of the plate surrounding the explant. Over this layer a population of small, round, phase-bright cells could be seen. Once the stromal cell layer became confluent and there was a large population of bright phase cells, the loosely-adherent cells surrounding the explants were harvested. This was performed by first washing the plate with Ca⁺⁺—Mg⁺⁺-free PBS, then with 0.48 mmol/L EDTA (for 1-2 min) and finally with 0.05% trypsin-EDTA (for 2-3 min). All washes were performed at room temperature under visual control to determine when the loosely adherent cells became detached. After each step the wash fluid was collected and pooled with that from the other steps. After the final wash, the explants were covered again with CEM and returned to the incubator. Each plate of explants may be harvested in this manner for up to four times at 5-10 day intervals. The pooled wash fluid was then centrifuged at 1000 rpm for 6-8 minutes, forming a cellular pellet. When centrifugation was complete, the supernatant was removed, the pellet was resuspended, and the cells were counted using a hemacytometer. The cells were then plated in poly-d-lysine coated 24-well tissue culture plates at a density ranging from 3-5×10⁴ cells/well (depending on the species) and returned to the incubator. The cells may optionally be grown in either “Cardiosphere Growth Media” (CGM) consisting of 65% Dulbeco's Modified Eagle Media 1:1 with Ham's F-12 supplement and 35% CEM with 2% B27, 25 ng/mL epidermal growth factor, 80 ng/mL basic fibroblast growth factor, 4 ng/mL Cardiotrophin-1 and 1 Unit/mL thrombin, or in CEM alone.

In either media, after a period of 4-28 days, multicellular clusters (“cardiospheres”) formed, detached from the tissue culture surface and began to grow in suspension. When sufficient in size and number, these free-floating cardiospheres were then harvested by aspiration of their media, and the resulting suspension was transferred to fibronectin coated tissue culture flasks in CEM (cells remaining adherent to the poly-D-lysine-coated dishes were not expanded further). In the presence of fibronectin, cardiospheres attached and formed adherent mono layers of “Cardiosphere-Derived Cells” (CDCs). These cells grow to confluence and then may be repeatedly passaged and expanded as CDCs, or returned to poly-d-lysine coated plates, where they will again form cardiospheres. Grown as CDCs, millions of cells can be grown within 4-6 weeks of the time cardiac tissue is obtained, whether the origin of the tissue is human, porcine or from rodents. When collagenase is used, the initial increase in cells harvested per mass of explant tissue results in faster production of large numbers of CDCs.

### Example 2

### Isolation of Porcine Cardiac-Derived Cells

Porcine CDCs were isolated and cultured according to Davis et al., (2009) PLoS One 4(9):e7195. Briefly, porcine endomyocardial specimens were sampled from the right ventricular septum using the bioptome. Biopsy specimens were stored on ice in high-potassium cardioplegic solution (5% glucose, 68.6 mM mannitol, 12.5 meq potassium chloride, 12.5 meq sodium bicarbonate, 10 units/mL heparin) to maintain tissue viability during transport. Tissue was processed within 2 hours. The myocardial specimens were then cut into fragments less than 1 mm³, washed and partially digested with collagenase. These tissue fragments were cultured as cardiac explants on fibronectin (20 μg/mL; Sigma) coated dishes in cardiac explant media (CEM; Iscove's Modified Dulbecco's Medium (GIBCO), fetal bovine serum (10% (mini swine only); HyClone, Logan, Utah), 100 U/ml penicillin G (GIBCO), 100 U/mL streptomycin (GIBCO), 2 mmol/L L-glutamine (Invitrogen, Carlsbad, Calif.), and 0.1 mmol/L 2-mercaptoethanol (GIBCO)). After a variable period of growth, a layer of stromal-like cells emerged from the cardiac explant over which phase bright cells proliferated. The loosely adherent cells surrounding the explant (termed cardiac outgrowth) were harvested using mild enzymatic digestion (0.05% trypsin (GIBCO) under direct visualization or no more than 2 minutes). Cardiac outgrowth could be harvested up to four more times from the same specimen. Cardiospheres were then cultured in CEM with 10% FBS. Several days later, cells that remained adherent to the poly-D-lysine coated dishes were discarded, while detached cardiospheres were harvested, plated on fibronectin coated flasks and cultured in CEM (10% or 20%, as indicated above) to generate CDCs.

### Example 3

### Labeling of CDCs With SPIO

SPIO microsphere particles (0.9-μm diameter, Bangs Laboratories, IN) were incubated with CDCs (1×10⁶ cells in 15 ml medium) in a T75 flask with a ratio of microspheres to cells at 500:1. As discussed herein, additional ratios of microspheres (or other labeled particle to cells may be used in some embodiments (e.g., 100:1; 250:1; 1000:1; 2000:1, 4000:1, etc.). The cells were incubated overnight at 37° C. and 95% air/5% CO₂ to incorporate SPIO into the cells.

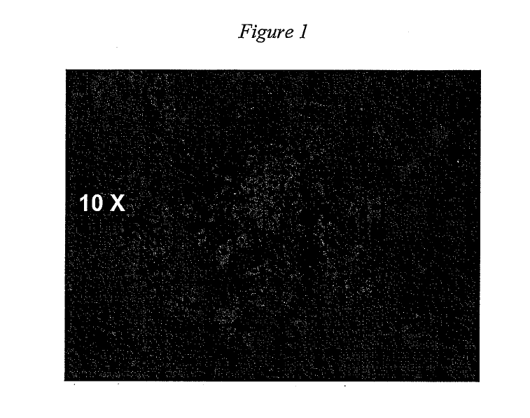

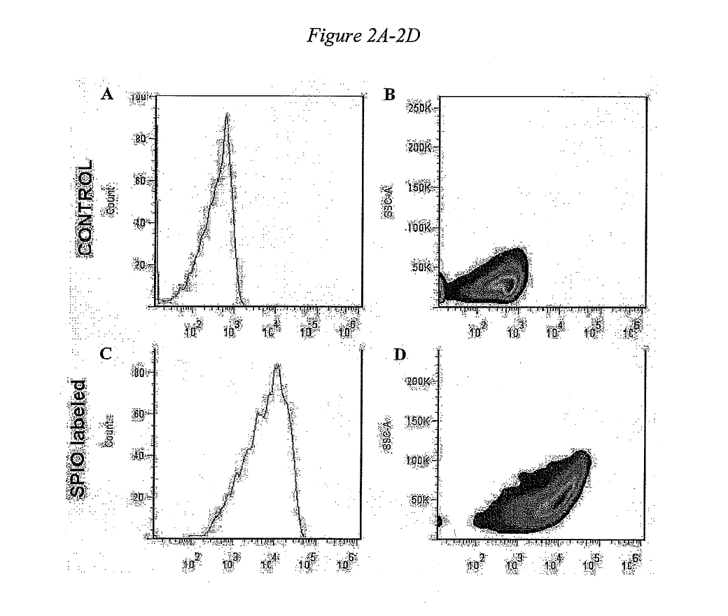

The labeling efficiency was assessed by microscopic examination and flow cytometry. CDCs labeled with SPIO (with a dragon green fluorescence tag) overnight were examined under a fluorescence microscopy (Excitation 488 nm; Emission 520 nm). A green color was observed, which indicates that the cells were successfully labeled with the SPIO (see FIG. 1). The labeling efficiency was also analyzed by flow cytometry. As shown in FIG. 2, compared to the control group (Panels A and B), the histogram shifts to the right hand side as SPIO-labeled cells exhibit a green fluorescence (Panels C and D).

### Example 4

### Retention and Myocardial Regeneration of Iron-Labeled Cdcs Transplanted into Injured Rat Hearts

CDCs were prepared as described in Example 1 and labeled with SPIO micro spheres at a 500:1 SPIO-to-cell ratio as described in Example 3. The labeling efficiency was 86%±1% (n=9), assessed by flow cytometry.

In vitro toxicity studies revealed that cellular functions (viability, apoptosis, attachment, proliferation, and antigenic phenotype) were minimally affected by SPIO labeling. Magnetically-labeled CDCs in a turbulent suspension were attached focally to the wall of test tubes under an externally applied 1 Tesla magnet

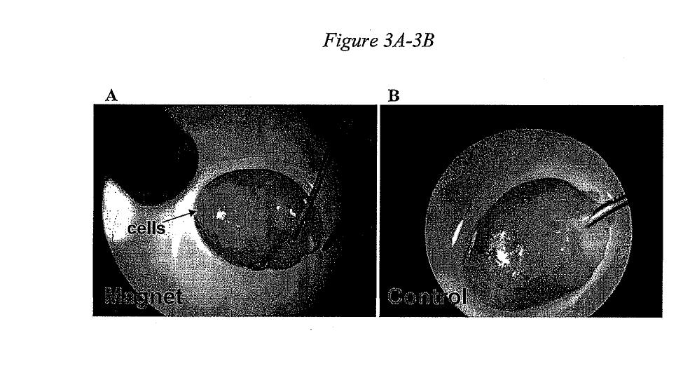

In vivo, female Wistar Kyoto (WKY) rats underwent left thoracotomy under general anesthesia, and MI was produced by permanent ligation of the left anterior descending coronary artery. The SPIO labeled-CDCs were administered to the WKY rats according to the protocol modified from Terrovitis et al. (2009) Journal of the American College of Cardiology 54(17):1619-1626, herein incorporated by reference. Briefly, about 1 million SPIO labeled-CDCs derived from syngeneic male WKY rats were injected intramyocardially (in 100 μL PBS as the vehicle) into the ischemic region, with and without a 1 Tesla NdFeB magnet applied approximately 1 cm above the apex of the heart for 10 min. Subsequently, the chest was closed. After 24 hours, examination of excised hearts indicated the animal exposed to the magnet had more cells present in the heart. As shown in FIG. 3, white light imaging revealed that SPIO-labeled cells, which have a dark brown color, were attracted towards the magnet and “trapped” around the infarct (labeled arrow), while the majority of nontargeted cells washed out immediately after injection.

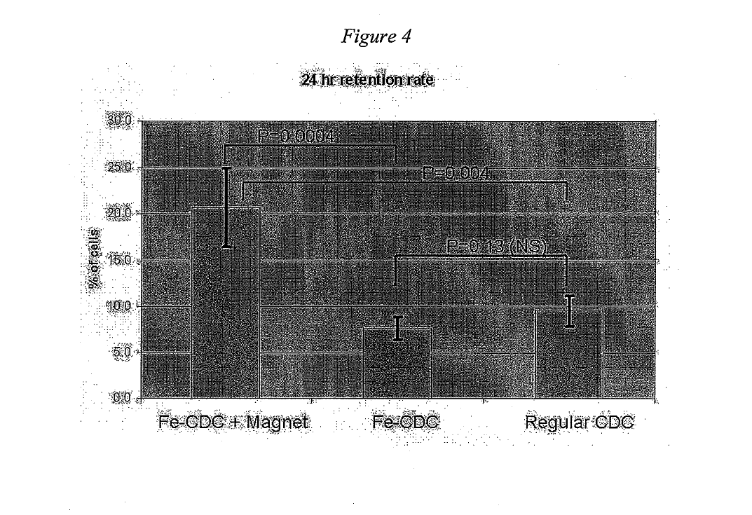

Polymerase chain reaction (PCR) analysis for the rat Y-chromosome-specific SRY gene was performed 24 hours and 3 weeks after cell transplantation. The DNA extraction was performed with a QIAamp Tissue Kit (Qiagen, Valencia, Calif.) according to the manufacturer's instructions. The primers and probe for rat SRY gene were: forward primer 5′ AGA GGC ACA AGT TGG CTC AAC 3′ and reverse primer 5′ TIC CAC TGA TAT CCC AGC TGC T 3′. PCR was done as previously described in Francois et al. (2006) Stem Cells. 24:1020-1029. Quantitative real-time PCR was performed using a sequence-detection system (Applied Biosystems) according to the manufacturer's instructions. PCR results confirmed that magnetic targeting enhanced cell retention rate in the recipient hearts. As shown in FIG. 4, targeted CDCs exhibited an approximately 3-fold enhancement of retention compared to nontargeted cells at 24 hour after injection (20.7±4.3% vs. 7.6±1.2%, n=7, p<0.0005). Overall, the retention rate of injected cells directly into injured hearts was improved with the application of an external magnet for a short period of time. Thus, in several embodiments, magnetic targeting of cells to a target tissue results in significantly increased short term retention of the cells within the target tissue. In some embodiments, this increase in short-term retention also yields an increase in long-term engraftment. However, in some embodiments, the increase in short term retention is sufficient to induce functional recovery and/or regeneration of cardiac tissue.

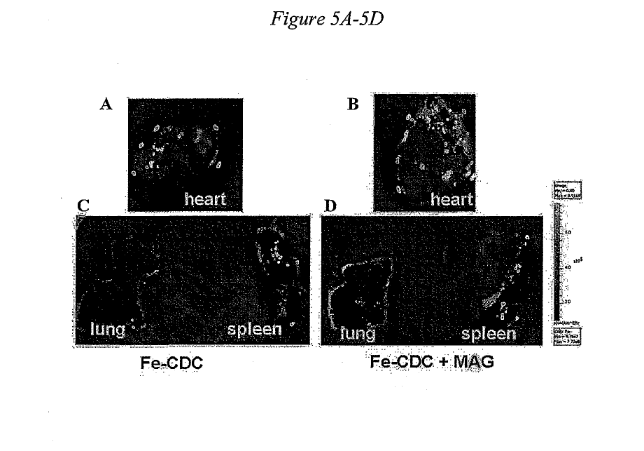

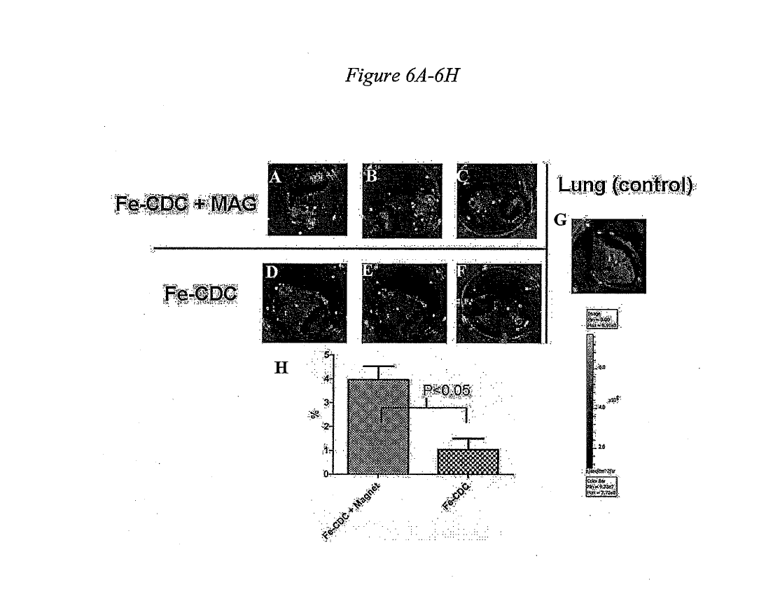

Fluorescence imaging (FLI) of excised hearts was performed to determine the levels of acute and long-term cell retention. Rat CDCs were labeled with SPIO (0.9 gm diameter, Bangs Laboratories, IN), which has a red fluorescence tag (excitation: 660 nm; emission: 690 nm). Cells were tracked with the IVIS 200 Fluorescence Imaging System (Xenogen) and signals were analyzed with the Living Image Software (Xenogen). A standard curve was generated by known cell dosage and background fluorescence was compensated for calculation. As shown in FIG. 5, at 24 hour after cell transplantation, FLI images revealed that the magnetic targeting group (Panels B and D) had more cell retention in the heart but less off-target expression in other organs (such as lung and spleen), compared to the non targeting group (Panels A and C). At 3 weeks after transplantation, FLI revealed that targeted CDCs exhibited approximately 4-fold enhanced of retention compared to non-targeted cells and no cells were found in the lungs after 3 weeks (see FIG. 6). Panels A, B, and C depict hearts of animals treated with labeled CDCs that were magnetically targeted while panels D, E, and F depict results of administration of labeled cells without magnetic targeting. Panel G shows limited off-target (liver) deposition of cells. Panel H indicates that the use of magnetic targeting evoked a significant increase in the retention of labeled cells at 3-weeks post administration. Thus, in several embodiments, magnetic targeting is advantageous because it not only increases the short term retention of delivered labeled cells (above), but because it significantly improves long term retention as well. In some embodiments, long term retention is advantageous to effect the structural and functional recovery of damaged target tissues (e.g., post-infarct cardiac tissue). However, increased long term retention is not a pre-requisite for such improvements. In some embodiments, long term retention of labeled cells is not significantly improved, however functional or physiological improvements in the target tissue are realized nevertheless. In such embodiments, the improvements may be due to the improved short term retention of the cells, or other beneficial attributes of magnetic targeting described herein.

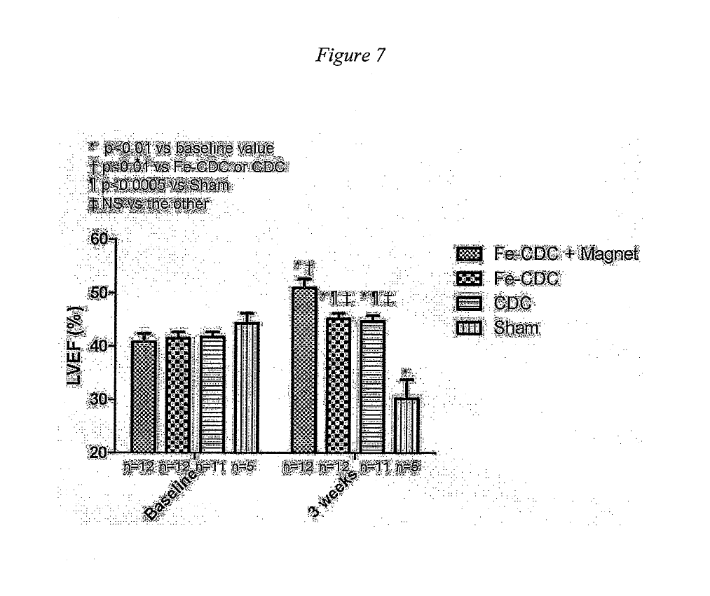

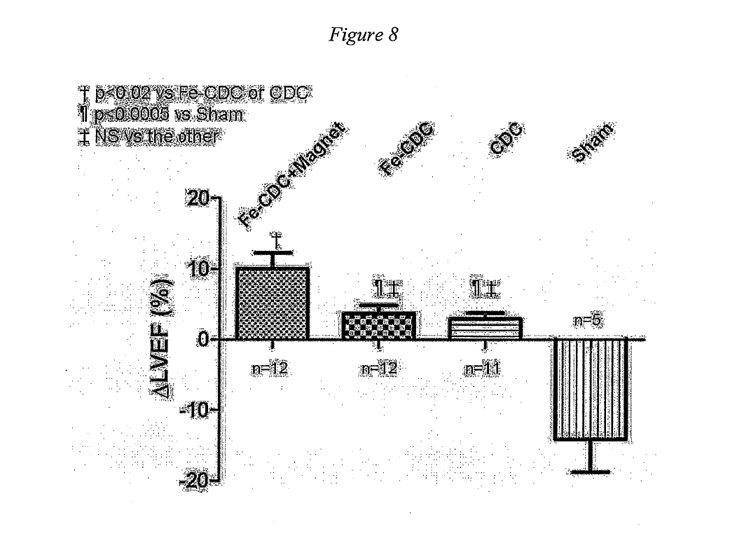

Cardiac function was measured on the day of cell transplantation (as the baseline) and 3 weeks after cell transplantation (as the end point). Ventricular performance was quantified by echocardiography (RMV-707B scan head, Vevo770, Visual Sonics). Ejection fraction (EF) (%) was calculated as [(LVVd-LVVs)/LVVdL100, where LVVd is left ventricular end-diastolic volume (L) and LVVs is left ventricular end-systolic volume (L). Ejection fraction is the fraction of blood pumped out of a ventricle with each heart beat, and can be useful in determining the capacity of the heart to pump blood to the body. The data generated showed that the heart function of SPIO-labeled and magnetically targeted CDCs significantly outperformed the two control groups: 1) SPIO-labeled CDCs without targeting; and 2) regular CDCs without labeling (see FIGS. 7 and 8). FIG. 7 depicts the baseline LVEF of sham treated, CDC treated, labeled CDC treated, and labeled CDC treated with magnetic targeting groups as well as the LVEF after 3 weeks. FIG. 8 depicts the change in LVEF detected after the 3 week period. After 3 weeks, all groups showed significantly different LVEF as compared to baseline. Groups receiving CDCs of any type showed improved LVEF, while sham animals showed a reduced LVEF. The effect of CDCs alone was not significantly different that labeled (but non targeted CDCs). However, labeled CDCs with magnetic targeting resulted in significantly increased LVEF, even as compared to CDC-receiving animals. As such, in some embodiments, administration of CDCs, whether native, labeled, or labeled and targeted, results in increased LVEF. In several embodiments, magnetically targeted cells yield an increase in the function of a damaged tissue. In some embodiments, magnetically targeted CDCs induce an increase in LVEF of at least 5%. In some embodiments, magnetically targeted CDCs induce an increase in LVEF of at least 10%. In some embodiments, greater increases in LVEF are detected. In some embodiments, an increase in LVEF is associated with significant overall improvement in cardiac function, cardiac output, and or quality of life. In some embodiments, increase LVEF is correlated with one or more of increased short or long term cell retention or increased engraftment.

### Example 5

### In Vitro Assays for Determination of CDC Properties Following Magnetic

### Particle Labeling

### Matrigel Angiogenesis Assay

Angiogenesis of CDCs will be assessed by Matrigel in vitro angiogenesis. Briefly, the gel solution is transferred to each well of a precooled tissue culture plate and incubated at 37° C. for at least one hour to allow the gel solution to solidify. CDCs will be harvested, resuspended in media and seeded onto the surface of the polymerized Matrigel. Next, CDCs will be incubated at 37° C. in the presence or absence of various concentrations of agents described above. Morphological change of the cells is observed at 4, 8 and 12 hours under an inverted light microscope. Patterns of CDCs will be recorded and compared with the initial CDC pattern throughout the experiment. The total capillary length and number of branching points are observed and quantified in several random view-fields (3-10) per well. Optionally, cells will be stained with commercially available cell stains such as Wright-Giemsa stain crystal violet, or Masson's trichrome to facilitate visualization of cellular networks.

### CDC Migration Assay

In vitro CDC migration will be performed using a modified Boyden chamber assay. Briefly, serum-starved CDCs will be loaded into the upper compartment of a 96-well microchemotaxis chamber where they will be allowed to migrate through the pores of a membrane (e.g., Matrigel coated PET membrane) into the lower compartment. Various concentrations of the agents described above will be added to the lower chamber. The membrane between the two compartments will be fixed and stained after 4, 8, 12, 18 and 24 hours. The number of cells that have migrated to the lower side of the membrane will be determined.

### CDC Survival Assay

In vitro CDC survival will be assessed by the WST-1 survival assay. The WST-1 assay is a colorimetric assay based on the cleavage of the tetrazolium salt WST-1 to formazan by cellular mitochondrial dehydrogenases. Cell proliferation results in an increase in the overall activity of the mitochondrial dehydrogenases in the sample, corresponding to an increase in formazan dye metabolism. Briefly, on day 1, WST-1 will be added to cells in the various groups described above. Cells will be incubated for 3-4 hours under normoxic or hypoxic conditions (1%, 2% or 4% O₂). The formazan dye produced by the viable cells will be measured at an absorbance of 440 nm using a standard multiwell spectrophotometer each day for up to one week. The extent of cell proliferation will be calculated relative to day 1, based on absorbance readings for each sample collected on each day.

### CDC Apoptosis Assay

The apoptosis of CDCs will be assessed using known methods, such as by terminal deoxy-nucleotidyl transferase mediated dUTP nick end-labeling (TUNEL) assay for labeling DNA breaks with fluorescent tagged deoxyuridine triphosphate nucleotides (F-dUTP) and total cellular DNA to detect apoptotic cells by flow cytometry or laser scanning cytometry. The enzyme terminal deoxynucleotidyl transferase (TdT) catalyzes a template independent addition of deoxyribonucleoside triphosphates to the 3′-hydroxyl ends of double- or single-stranded DNA. In brief, CDCs treated in the various groups described above will be washed with buffer, resuspended, and added to a microtiter plate. Fresh 4% paraformaldehyde in PBS will be added to the cells, which are then incubated 30 minutes at room temperature on a shaker. Subsequently, the plate will be centrifuged for 10 minutes and the supernatant is removed. Cells will be resuspended in permeabilization buffer and incubated with TUNEL reaction mixture for an hour at 37° C. until analysis.

### Example 6

### Contemplated Methods for Administration of Vascular Permeability Agents and CDCs in Mouse Infarction Model

Male C57B1/6 mice 22-28 g (Jackson Laboratory) will undergo anesthesia, analgesia, tracheal intubation, pulmonary ventilation (2 cm H₂O pressure, 120 min⁻¹, IITC Life Science, Woodland Hills, Calif.), intercostal thoracotomy and ligation of the left anterior descending (LAD) coronary artery (7-0 monofilament suture, Ethicon) to create experimental myocardial infarction. The mice will be separated into groups receiving one of the following treatment regimens injected into the coronary artery, or alternatively the myocardium, immediately after ligation:

Group 1—VEGF165 plus SPIO-labeled CDCs with magnetic attraction.

Group II—serotonin plus SPIO-labeled CDCs with magnetic attraction.

Group III—nitroglycerin plus SPIO-labeled CDCs with magnetic attraction.

Group IV—SPIO-labeled CDCs alone with magnetic attraction.

Group V—unlabeled CDCs alone with magnetic attraction.

Group VI—SPIO-labeled CDCs alone without magnetic attraction.

Group VII—unlabeled CDCs alone without magnetic attraction.

A sham surgery control group, will undergo all procedures described except ligation of the LAD. ECG and rectal temperature will be monitored intra-operatively. The animals will be recovered overnight in a 37° C. environment. The surgeries will be performed as part of an institutionally approved protocol. The animals will be euthanized at 2, 7 or 14 days. (n=5 for MI and sham groups, at each time point) for harvest of cardiac tissue. Alternatively, the animals will be monitored for a period of days following injection, for example, by echocardiography (e.g., to measure left ventricular end systolic dimension (LVESD), left ventricular end diastolic dimension (LVEDD), fractional shortening (FS=100xLVEDD-LVESD/LVEDD) and heart rate) or magnetic resonance imaging (MRI) (e.g., to track labeled cells in the cardiac tissue, to measure left ventricular volumes at end systole and end diastole (LVESV, LVEDV), left ventricular mass (LV mass), left ventricular ejection fraction (LVEF=LVED−LVESV/LVEDV×100), and left ventricular wall thickening.

The removed cardiac tissue will be subjected to routine histological or alternatively, immunocytochemical analysis. For example, in one embodiment, the cardiac tissue can be fixed and vibratome sectioned to 1 mm-5 mm thickness, and the resulting sections uniformly processed and paraffin embedded for histology. Some of the sections will be stained with hematoxylin-eosin and picrosirius red/fast green to determine, e.g., infarct size. Immunohistochemistry can be performed, e.g., with antibodies directed to various muscle antigens, cardiac antigens or other cell-type antigens.

The animals in Group I-III will have improved delivery and retention rate, cell engraftment and cardiac function as compared to Groups IV (labeled CDCs alone), which will have improved delivery and retention rate, cell engraftment and cardiac function as compared to one or more of Groups V (unlabeled CDCs alone), Group VI (SPIO-labeled CDCs alone without magnetic attraction) and Group VII (unlabeled CDCs alone without magnetic attraction).

### Example 7

### Contemplated Methods for Administration of Vascular Permeability Agents and Human CDCs in SCID Mouse Infarction Model

Myocardial infarction will be created by ligation of the LAD coronary artery in the SCID mice. Human CDCs will be prepared, cultured and labeled using protocols described above. Immediately after LAD ligation, one of the following treatment regimes will be administered to the mice according to their assigned groups:
Group I—intracardiac injection of 10⁵ human SPIO-labeled CDCs in 10 μL PBS with magnetic attraction.
Group II—intraperitoneal injection of 50 μg VEGF165, sildenafil, serotonin or nitroglycerin in 300 μL PBS (optionally repeated every 3 days for up to 2 weeks).
Group III—intracardiac injection of 10 μg VEGF165, sildenafil, serotonin or nitroglycerin in 100 μL PBS (optionally repeated every 3 days for up to 2 weeks).
Group IV—treatment regimes of Group I plus Group II.
Group V—treatment regimes of Group I plus Group III.
Group VI—intracardiac injection of 10 μl PBS and intraperitoneal injection of 300 μl of PBS at the time of surgery (optionally repeated every 3 days for up to 2 weeks).

### Functional Evaluation

The cardiac functional evaluation of experimental mice will be assessed by mouse echocardiography in awake or anesthetized mice with chest hair removed at day 1, weeks 3 and 6 post-MI. Limb leads will be attached for electrocardiogram gating, and the animals will be imaged in the left lateral decubitus position with a 13-MHz linear probe. Two-dimensional images will be recorded in parasternal long- and short-axis projections with guided M-mode recordings at the midventricular level. Left ventricular cavity size and wall thickness will be measured at least three beats from each projection and averaged. Left ventricular end systolic dimension, fractional area shortening, LV fractional shortening, relative wall thickness, LV mass, ejection fraction will be calculated.

### Human Cell Graft Size

Human CDC graft size will be measured by real-time PCR at weeks 3 and 6 following MI procedure using human specific Alu probe. The CDC graft size will be assessed by the abundance of Alu, which will be quantified using real-time PCR and a standard curve generated by control samples with known number of human CDCs (e.g., 10² to 10⁵) per 12.5 grams of mouse heart tissue.

### Histological Evaluation

The degree of fibrous tissue will be assessed at 3 and 6 weeks post MI procedure using Masson's trichrome stain. The degree of apoptosis will be assessed using a TUNEL assay at 24 hours post-MI procedure. Finally, the degree of inflammatory cell infiltration will be assessed using a myeloperoxidase assay at 24 hours post-MI procedure.

### Example 8

### Methods for Analysis of Myocardial Regeneration

Horizontal cryosections of 14 μm thickness spaced at 1 mm intervals will be analyzed. To determine infarct size, Masson's Trichrome-stained sections will be analyzed at 1× magnification. The infarct border zone is defined as myocardial tissue within 0.5 mm of the fibrous scar tissue. Fibrosis and cardiomyocyte cross-sectional area will be determined after staining with Masson's Trichrome at 10× and 40× magnification, respectively, and quantified using the Metamorph software package. BrdU-positive cardiac fibroblast nuclei will be determined at 5 cross-sections per heart at the level of the myocardial infarction. Cardiomyocyte nuclei will be counted using the optical dissector method (Howard, CV. & Reed, M. Unbiased Stereology: Three-Dimensional Measurement In Microscopy, (BIOS Scientific Publishers, Oxford, 2005)) on troponin T and DAPI-stained sections in 32-60 random sample volumes of 84,500 μm per heart. BrdU-positive cardiomyocyte nuclei will be quantified on 16-20 sections per heart. Cardiomyocyte apoptosis will be determined using the In situ Cell Death Detection Kit (Roche) in combination with staining for troponin I. Capillaries, arterioles, and stem cells are detected with antibodies against von Willebrand factor (vWF), smooth muscle actin (SMA), and c-kit, respectively, and quantified at the level of the myocardial infarction.

### Example 9

### Contemplated Methods for Administration of Vascular Permeability Agents and CDCs in Rat Model of Myocardial Infarction

Adult male Sprague-Dawley rats (300 gm, Charles River Laboratories) will undergo experimental myocardial infarction as described (del Monte. et al. (2004) Proc Natl Acad Sci USA 101, 5622-7). The survival rate is generally about 67%. GELFOAM® loaded with SPIO-labeled CDCs (10⁵-10⁹) and simultaneously with 100 μg of the following combinations of vascular permeability agents: (i) VEGF 165 and serotonin; (ii) serotonin and nitroglycerin; (iii) VEGF165 and nitroglycerin; or (iv) buffer alone, will be applied over the myocardial infarction at the time of surgery in the presence and absence of magnetic actuation. Rats will receive 3 intraperitoneal BrdU injections (70 μmol/kg body weight) with a half-life of 2 hr every 48 hr over a period of 7 days. Echocardiography and hemodynamic catheterization will be performed as described (Prunier et al. Am J Physiol Heart Circ Physiol (2006)).

### Example 10

### Contemplated Administration of Vascular Permeability Agents and CDCs in Porcine Myocardial Infarction Model

The porcine myocardial infarction will be created according to Zuo et al. (2009) Acta Pharmacologica Sinica 30:70-77. Briefly, pigs will be anesthetized with intramuscular diazepam (0.05 mg/kg), atropine (0.05 mg/kg), ketamine (20 mg/kg), intubated. A limited left thoracotomy will be performed in a sterile condition through the fifth intercostal space with a small incision in the pericardium. The porcine heart will be exposed and suspended in a pericardial sling. A silk suture will be set at ⅓ marginal branch of the left anterior descending (LAD) coronary artery and ligated 20 min later. Coronary occlusion will be confirmed by the presence of raised ST stages on the electrocardiogram and ventricular arrhythmias within the 1st 20-30 min after occlusion.

GELFOAM® loaded with SPIO-labeled CDCs (10⁵-10⁹) and simultaneously with 100 μg of the following combinations of vascular permeability agents: (i) VEGF165 and serotonin; (ii) serotonin and nitroglycerin; (iii) VEGF165 and nitroglycerin; or (iv) buffer alone, will be applied over the myocardial infarction at the time of surgery, under an external magnetic application. Pigs will receive 3 intraperitoneal BrdU injections (70 μmol/kg body weight) in the presence and absence of magnetic actuation with a half-life of 2 hr every 48 hr over a period of 7 days. Echocardiography and hemodynamic catheterization will be performed as described (Prunier et al. (2006) Am J Physiol Heart Circ Physiol 292(1):H522-9).

### Example 11

### Contemplated Administration of Adenosine, Fibrin Glue or BDM and CDCs in Rat Model of Myocardial Infarction

Female Wistar Kyoto (WKY) rats will undergo left thoracotomy under general anesthesia, and MI will be produced by permanent ligation of the left anterior descending coronary artery. Approximately 2 million of SPIO-labeled CDCs derived from male WKY rats will be injected directly into the myocardium, at 2 sites into the infarct according to the following groups:
Group I—CDCs will be injected intramyocardially after the induction of cardiac arrest.
Group II—CDCs will be lysed with sonication after labeling and before injection.
Group III—CDCs will be suspended in PBS and injected intramyocardially.
Group IV—CDCs will be suspended in PBS containing 100 mmole/L of 2,3-butanedione-2-monoxime (BDM) to locally suppress contractility at the injection site.
Group V—after intramyocardial cell injection, the epicardial side of the injection site will be sealed by fibrin glue (FG).
Group VI—intramyocardial delivery of cells will be performed during slowing of ventricular rate by intravenous injection of adenosine (1 mg).
Group VII—cell delivery will be performed during intravenous adenosine injection and subsequently the injection site is sealed epicardially by FG.

Quantitative PCR will be performed at 1 hour and 21 days after cell injection to compare medium term engraftment in these groups. Engrafted donor cell numbers will be quantified as a function of time, by real-time PCR, with the SRY gene located on the Y chromosome as target according to Terrovitis et al. (2009) Journal of the American College of Cardiology 54(17):1619-1626, herein incorporated by reference. Echocardiography will be performed with the Vevo 770 system (Visualsonics, Toronto, Canada) to assess global cardiac function.

### Example 12

### Contemplated Methods for Assessment of Magnetism to Promote Cardiac Retention of Iron-Loaded Cells in a Porcine Model

Animals will be divided into the following groups:
Group 1: Donor male pigs. Hearts from the donor male pigs will be harvested. CDCs derived from the cardiac tissues will be used in subsequent experiments.
Group 2: Recipient female pigs with a normal heart. CDCs will be delivered to the heart via coronary infusion with application of a magnet.
Group 3: Recipient female pigs with a normal heart. CDCs will be delivered to the heart via coronary infusion of cells without application of a magnet.
Group 4: Recipient female pigs with acute myocardial infarction induced by balloon occlusion of the LAD coronary artery, followed by coronary infusion of cells with application of a magnet.
Group 5: Recipient female pigs with acute myocardial infarction induced by balloon occlusion of the LAD coronary artery, followed by coronary infusion of the cells without application of a magnet.

### Group 1

DCs derived from pigs of Group 1 will be used in subsequent experiments performed in animals from Groups 2-5. Briefly, pigs in Group 1 will be fasted for 18 hours prior to surgery, and sedated/immobilized with intra-muscular drugs (acepromazine, ketamine and atropine). An IV cannula will be inserted into an ear vein. Once immobilized, the animals will be taken to the necropsy room and euthanized by intravenous infusion of a veterinary euthanasia solution and sacrificed. Subsequently, the chest cavity will be opened and the heart will be harvested for preparation of iron-loaded cells (SPIO-labeled cells) according to the methods disclosed herein.

### Groups 2 and 3

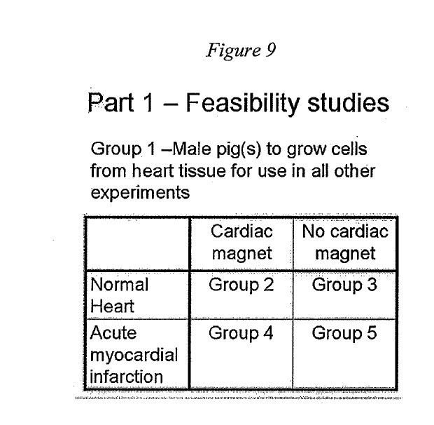

The recipient pigs in Groups 2 and 3 will receive intracoronary infusion of CDCs derived from pigs in Group 1. A summary of this contemplated study is provided in FIG. 9. Briefly, animals will be fasted for 18 hours prior to surgery and sedated/immobilized with intra-muscular drugs (acepromazine, ketamine and atropine). An IV cannula will be inserted into an ear vein, followed rapidly by the induction of anesthesia with intravenous thiopental. Once anesthetized, the trachea will be intubated to provide a secure and open route for ventilating the lungs. The hair on the neck will be clipped and the anesthesia will be maintained by 1-3% inhaled anesthetic gas (isoflurane). Surgical cut-down to the left carotid artery will then be performed. Coronary artery catheters will be inserted into the artery and anticoagulation via intravenous heparin will be given. Coronary angiography will be performed. An image of the artery will be acquired by injection of a dye that can be visualized by X-ray.

Subsequently, iron-loaded cells derived from Group 1 will be infused into the coronary arteries. For Group 2, cells will be suspended in 10 mL of high permeability solution (optimized for coronary cell infusion during previous experiments) for infusion over several minutes, and the heart will be subject to a magnetic field by placing a 5 inch external magnet over the heart region. The magnet will be left in place for approximately 20 minutes. Group 3 will be the control group for comparison. Pigs in Group 3 will receive coronary infusion of the iron-loaded cells in the same solution without application of a magnet.

The intracoronary cell infusion will be carried out according to the following procedure. Briefly, the cell suspension will be administered in 3 divided doses of 3.3 mL each. The cells will be infused through the central lumen of an angioplasty balloon placed in the coronary artery. During cell infusion, the angioplasty balloon will be inflated for 3 minutes to prevent the infused cells from being washed away by flowing blood. Between doses, the balloon will be deflated for 3 minutes to allow coronary flow. At the conclusion of coronary cell infusion, the coronary balloon and catheter will be removed.

A delay of 1 hour will be scheduled. The pigs will remain under anesthesia, but no active procedures will be performed. Euthanasia will subsequently be performed, by increasing the level of anesthesia by increasing the inhaled anesthetic drug (isoflurane) to 4%. An overdose of intravenous potassium chloride will be given to stop the heart beating. Sacrificed animals will be taken to the necropsy room where the hearts will be removed from the chest. The lungs, kidneys, livers, and spleens will also be removed for examination. The hearts will be scanned in the clinical MRI machine to detect iron loaded cells, and the hearts will also be subjected to pathological examination. These assessments will be used to measure the amount and location of retained iron within the heart muscle and evaluate the cell retention rate. Lungs, kidneys, liver and spleen will be examined in the laboratory for “off-target” deposition of iron-labeled cells. The examinations will include specific laboratory tests to determine the iron content, which may include Perls' stain, Prussian blue stain and/or the dry weight percentage iron test according to Barry and Sherlock (1971) Lancet 1:100-103.

### Groups 4 and 5

Recipient animals in Groups 4 and 5 will receive coronary cell infusions into the hearts following acute myocardial infarction. The animals will be fasted for 18 hours prior to surgery. Carprofen (oral pain-killer) will be administered to the animals on the morning of surgery. The animals will be sedated/immobilized with intra-muscular drugs (acepromazine, ketamine and atropine), with an insertion of IV cannula into an ear vein, followed rapidly by the induction of anesthesia with intravenous thiopental. Once anesthetized, the trachea will be intubated to provide a secure and open route for ventilating the lungs. The hair on the neck will be clipped, and the skin will be prepared with betadine. The anesthesia will be maintained by 1-3% inhaled anesthetic gas (isoflurane). An anti-arrhythmic drug, amiodarone, will be administered intravenously. Surgical cutdown to the left carotid artery will be performed. Coronary artery catheters will be inserted into the artery and intravenous heparin will be given for anticoagulation. The coronary angiography will be performed. An image of the artery will be acquired by injection of a dye that can be visualized by X-ray. A balloon catheter will be inserted into the left anterior descending (LAD) coronary artery and inflated once only to occlude blood flow for 150-180 minutes. This will produce infarction (death) of the cardiac muscle supplied by the LAD artery. The balloon will then be deflated and removed from the body.

The iron-loaded cells derived from Group 1 will be infused into the coronary arteries. For Group 4, cells will be suspended in 10 mL of high permeability solution (optimized for coronary cell infusion during previous experiments) for infusion over several minutes, and the heart will be subject to a magnetic field by placing a 5 inch external magnet over the heart region. The magnet will be left in place for 20 minutes. The animals in Group 5 will receive coronary infusion of the iron-loaded cells in the same solution without application of a magnet. Intracoronary cell infusion will be performed as in Groups 2 and 3. At the conclusion of coronary cell infusion the coronary balloon and catheter will be removed. A delay of 1 hour will be subsequently scheduled. The pigs remain under anesthesia, but no active procedures will be performed. Euthanasia will be performed, and the level of anesthesia will deepened by increasing the inhaled anesthetic drug (isoflurane) to 4%. An overdose of intravenous potassium chloride will be given to stop the heart beating. The animals will then be taken to the necropsy room where the hearts will be removed from the chest. The lungs, kidneys, livers, and spleens will also be removed for examination. The hearts will be scanned in the clinical MRI machine to detect iron loaded cells, and subject to pathological examination as in Groups 2 and 3 to measure the amount and location of retained iron, and to evaluate the cell retention rate.

### Example 13

### Contemplated Randomized Functional Study of Intracoronary Infusion of Iron-Loaded Cells In A Porcine Model

Female mini-pigs will be randomized to Group 6, 7 or 8 as follows:
Group 6 (Control) —intracoronary infusion of saline and application of a magnet to the heart.
Group 7—intracoronary infusion of iron-loaded cells, while a magnetic field is applied to the heart. Each animal will receive its own cells.
Group 8—intracoronary infusion of iron-loaded cells, Each animal will receive its own cells.

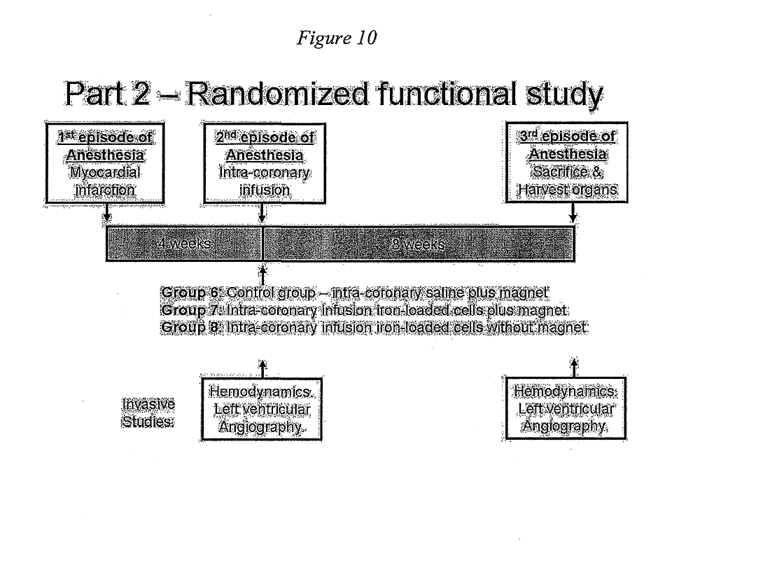

A summary of this contemplated study is provided in FIG. 10. Briefly, animals will be fasted for 18 hours prior to surgery. Carprofen (oral pain-killer) will be administered to pig on the morning of surgery. The animals will then be sedated/immobilized with intra-muscular drugs (acepromazine, ketamine and atropine) with insertion of IV cannula into an ear vein, followed rapidly by the induction of anesthesia with intravenous thiopental. Once anesthetized, the trachea will be intubated to provide a secure and open route for ventilating the lungs. The hair on the neck will be clipped, and the skin will be prepared with betadine. The anesthesia will be maintained by 1-3% inhaled anesthetic gas (isoflurane). An anti-arrhythmic drug, amiodarone, will be administered intravenously. Surgical cutdown to the left carotid artery will be performed. Coronary artery catheters will be inserted into the artery. Surgical cut-down to the left jugular vein will be also performed. The biopsy forceps will be introduced into the right ventricle of the heart via the vein and the anticoagulation with intravenous heparin will be given. Coronary angiography will be performed. An image of the artery will be acquired by injection of a dye that can be visualized by X-ray.

A balloon catheter will be inserted into the left anterior descending (LAD) coronary artery and inflated once only to occlude blood flow for 180 minutes. This will produce infarction (death) of the cardiac muscle supplied by the LAD artery. The balloon will then be deflated and removed from the body. Just after the infarction has been created, multiple cardiac biopsy specimens (up to 10) will be taken from the right ventricular septum using a standard clinical bioptome via the jugular vein to obtain a small amount of cardiac tissue; from these tissue specimens, cardiac stem cells will be grown in culture. The cells will be induced to take up tiny iron particles in the laboratory. The cells will be injected back into the coronary artery of the same pig 4-5 weeks later. All catheters will be removed, carotid artery will be repaired and jugular vein will be ligated, cutdowns are sutured and animals will be allowed to recover. Suture will be used to do interrupted sutures in the muscle and fascia and sub-cuticular stitch in the skin. The animals will be taken to post-op recovery room. Pain-killer (buprenorphine) will be given via intra-muscular injection after the surgery is completed, as the animals wake up from anesthesia.

Four to five weeks later, the animals in Groups 6, 7 and 8 will undergo a second episode of anesthesia and intubation as described above (fasting/oral carprofen pre-op/1M acepromazine, ketamine and atropine/IV thiopental/tracheal intubation/inhalational isoflurane). The hair on the neck and the groin will be clipped, and the skin will be prepared with betadine. Surgical cut-down to the left carotid artery and the jugular vein will be performed. Coronary artery catheters will be inserted into the artery and the anticoagulant heparin will be given intravenously. Left ventriculography will be performed to assess cardiac function. Pressure and volume measurements will be taken within the left ventricle to allow heart function to be accurately measured. Subsequently, the animals will receive an intracoronary infusion, which varies according to the experimental group. Group 6 will receive saline alone, with application of a magnet to the heart. Group 7 will receive infusion of cells grown from their own cardiac biopsy, along with application a magnet to the heart. Group 8 will receive infusion of cells grown from their own cardiac biopsy, but without application of a magnet to the heart.

Eight to nine weeks later (12-14 weeks after myocardial infarction), the animals will have a final measurement of cardiac function, followed by harvest of the heart and other organs for MRI and iron measurements.

### Example 14

### Contemplated Methods for Administration of Labeled CDCs in Human Subjects

Patients with chronic or acute heart failure will be given the following clinical procedures upon experiencing symptoms of myocardial infarction.

Catheterization will be performed by (A) intracoronary doppler (optional) followed by (B) coronary angiography and cell/agent administration. Doppler measurements and coronary angiography will be repeated in case that a premature coronary angiography has to be performed for clinical reasons (e.g., restenosis).

### Intracoronary Doppler

### Adenosine Administration

Adenosine (ADENOSCAN®) will be administered intravenously to the patient at a concentration of 140 μg/kg body weight/min at an infusion rate>100 ml/h according to the infusion scheme presented in Table 3 (or other similar approved protocol):

TABLE 3
Infusion Rate Conversions

| Body Wt. (kg) | ml/min | ml/h | Body Wt. (kg) | ml/min | ml/h |
|---|---|---|---|---|---|
| 45-49 | 2.1 | 126 | 85-89 | 4.0 | 240 |
| 50-54 | 2.3 | 138 | 90-94 | 4.2 | 252 |
| 55-59 | 2.5 | 150 | 95-99 | 4.4 | 265 |
| 60-64 | 2.8 | 168 | 100-104 | 4.7 | 282 |
| 65-69 | 3.0 | 180 | 105-109 | 4.9 | 294 |
| 70-74 | 3.3 | 198 | 110-114 | 5.1 | 306 |
| 75-79 | 3.5 | 210 | 115-119 | 5.4 | 324 |
| 80-84 | 3.8 | 228 | | | |

### Measurement of Flow Reserve in Infarct Artery

Flow reserve in the infarct artery will be measured according to the following procedure. First, the vessel will be pretreated with Nitroglycerin 0.2 mg i.c. FLOWIRE® will be positioned at the site of the stent (target lesion of index infarction), in which the position will be documented by coronary angiography. Adenosine infusion will begin following documentation of time, heart rate, blood pressure, and APV and will continue for further 45 seconds after maximal increase of flow (steady state). Bradycardia will be attended to during the time of infusion.

Measurement of Flow Reserve in Reference Vessel—Flow reserve in a reference vessel will be measured by the following procedure. First, the vessel will be treated with Nitroglycerin 0.2 mg, if not already performed in this vessel. FLOWIRE will be positioned at the site in a non-diseased portion of the vessel. In this procedure, an ideal reference vessel may be a vessel that has not been treated by PCI within the last 6 months, is not significantly diseased and has no previous myocardial infarction in the reference vessel. For this procedure, all three major vessels (RCA, LCX, LAD) or major branches may be suitable as a reference vessel. The coronary flow velocity will be attended to until it is back to baseline. This procedure will be repeated as described above for infarct artery. Angiographic projections will be documented for follow-up measurements.

### Preparation and Administration of CDCs

Percutaneous right ventricular endomyocardial biopsy specimens will be obtained from patients during previous hospital visits after informed consent using an institutional review board-approved protocol. SPIO-labeled CDCs will be prepared from the specimen and cultured according to protocols described above. Autologous CDCs will be administered to the patient following one of the treatment regimes, each performed with or without vascular permeability agent(s):
Group I—SPIO-labeled CDCs with magnetic attraction (magnetic field will be applied over the heart region for twenty minutes upon and/or following infusion of the labeled cells).
Group II—SPIO-labeled CDCs without magnetic attraction.
Group III—Unlabeled CDCs with magnetic attraction (see Group I).
Group IV—Unlabeled CDCs without magnetic attraction.

### Premedication

Prior to application of the above treatments, REOPRO® (Abciximab, bolus only) will be given to the patient according to the prescribed dosage of 0.25 mg/kg body weight over 1 min, with optional subsequent continuous infusion of abciximab at the discretion of the investigator.

Glycoprotein-receptor blocker therapy will be recommended at the time of treatment of the acute myocardial infarction by the protocol. However, indication and type of glycoprotein receptor blocker (tirofiban, eptifibatide or abciximab) will be left at the discretion of the physician in charge. Nevertheless, in line with current evidence, use of REOPRO® will be encouraged also at the index PCI. In this case, abciximab will be given as an re-administration during cell therapy. The platelet count will be controlled 6 and 24 hours after study therapy as well as prior to hospital discharge for any potential thrombocytopenia. In addition, approximately 50-70 units/kg of heparin will be given (target ACT 250-300s) prior to cell/placebo medium therapy.

### Balloon Placement

Balloon placement will be performed using a 6 F guiding catheter. For cell infusion, a conventional over-the-wire balloon catheter (e.g., OPENSAIL®, Guidant) will be used; cell- or placebo-solution will be infused through the central guide wire lumen. The balloon will be oversized by 0.5 mm compared to the size of the implanted stent to achieve an occlusion of the vessel during low pressure balloon insufflation. Long balloons with a length of 10 mm will be used in the procedure. However, if the balloon size is larger than 4 mm, only a 20 mm long balloon is available. Next, a conventional guide wire will be inserted in the OPENSAIL® balloon catheter (no long exchange wire will be necessary) to advance the balloon to the guide wire tip. The guide wire will be then introduced to the infarct vessel. Subsequently, the OPENSAIL® balloon catheter will be advanced to the previous infarct lesion; the balloon will be positioned within the stent.

### Set Up of Infusion

The infusion will be set up by retracting the guide wire and connecting a 3-way tap to the central lumen and the air will be removed from the system before injecting the cells. The central lumen will then be flushed with albumin, which lubricates the wall of the balloon catheter and avoids attachment of cells to the wall of the balloon catheter. The syringe containing CDCs according to the treatment regimes or placebo solution will be connected with the cell suspension to the 3-way tap.

### Balloon Insufflation and Cell Injection

Balloon insufflation will be performed according to the following procedure. Prior to and after this balloon inflation, the patient will be given 100 m adenosine i.v. in repeated boluses up to 1 mg. The vessel will be occluded with a low pressure balloon insufflation. It is important that a slightly oversized balloon is chosen to prevent the balloon pressure from exceeding 2-4 bars. A small amount (e.g., 1-ml) of contrast agent will be injected with care not to damage the occluded artery, in order to document that the vessel is actually occluded before giving the cells. A complete occlusion by coronary angiography will be recorded. If the vessel fails to be occluded by the above procedure, the balloon will be expanded and held at the 2-4 bar pressure. If the vessel still fails to become occluded despite adequate balloon expansion, a larger balloon will be used with care not to exert extensive pressure (>4 bar) on vessel wall. Injection of the progenitor cells will be allowed only if complete occlusion has been successfully documented by cine angiography. Balloon occlusion will be used to avoid wash out of the cells and to give the cells time to attach in the target area. The artery will be occluded for 3 minutes. Occlusion will be checked immediately by angiography; long delays between balloon occlusion and actual start of infusion of the study therapy will be avoided to maximize time for cells to home in the infarct area. Thereafter, one-third of the solution in the syringes (3.3 ml) will be injected within 10 seconds. The balloon will be deflated after 3 minutes. In case of severe angina pectoris, the balloon might be deflated earlier. However, patients after myocardial infarction can generally tolerate a three-minute occlusion without or with only minor chest pain. The actual time of sufficient balloon inflation after infusion of the cells will be documented. Three minutes after deflation of the balloon, this procedure will be repeated for two additional times. Finally, the balloon catheter will be removed; the integrity of the infarct artery by coronary angiography will be recorded. An overview angiography (RAO 30°; LAO 60°) will be performed additionally without zoom for documentation of the absence of microembolization. The schedule to be used for cell infusion is summarized in Table 4.

TABLE 4
Cell Infusion Time-table

| | |
|---|---|
| Balloon inflation | |
| Angiography to document occlusion | immediately after sufficient inflation |
| Infusion of cells | immediately after angiography |
| | (about 10 sec.) |
| Deflation of balloon | after 3 minutes |
| Pause | 3 minutes |
| Second balloon inflation | time schedule as above |
| Pause | 3 minutes |
| Third balloon inflation | time schedule as above |

The clinical procedures presented in this example will be given to the patients repeatedly over a course of one year at the discretion of the physician. Echocardiogram, cardiac MRI, a 24-hour Holter monitor and laboratories (including, e.g., complete blood count (CBC), blood urea nitrogen (BUN), creatinine, troponin, lactate dehydrogenase (LDH), c-reactive protein (CRP), and norepinephrine) will be performed periodically to assess adverse outcome.

### Example 15

### Contemplated Methods for Preparation of SPIO-Labeled CDC Unit Dosage Form

SPIO-labeled CDCs formulated in unit dosage will be prepared according to the following protocol. Briefly, CDCs will be obtained and cultured according to the methods described herein and labeled with SPIO microsphere particles according to the methods described herein. The labeled CDCs will be centrifuged at 1000 rpm for 6-8 minutes, forming a cellular pellet. The pellet will then be re-suspended in CEM medium supplemented with 10% to 20% fetal bovine serum or calcium-free PBS, and divided into sterile vials with 1×10⁵, 1×10⁶, 1×10⁷, 1×10⁸, 1×10⁹, 1×10⁷ of total counts of labeled CDCs per vial. Each vial of labeled cells will then be ready for single use, optionally with 100 μg VEGF 165, serotonin and/or nitroglycerin.

The cell pellet may also optionally be re-suspended in freezing media supplemented with 50% of DMSO with equal amount of DMSO slowing added into the CEM media. The labeled cells will subsequently be dispensed into sterile freezing vials in a total count of 1×10⁵, 1×10⁶, 1×10⁷, 1×10⁸, 1×10⁹, 1×10⁷ per vial, and frozen in liquid nitrogen.

### Example 16

### Magnetic Targeting Enhances Engraftment and Functional Benefit of Iron-Labeled CDCs After Myocardial Infarction

As discussed, above, stem cell transplantation is a promising therapeutic strategy for acute or chronic ischemic cardiomyopathy. However, low cell retention and limited engraftment present major obstacles to achieving a significant functional benefit. These limitations are troublesome to successful therapy irrespective of the cell type or delivery model used. Acute (e.g., ≦24 hour) retention of administered cells is typically less than 10%, regardless of the delivery route. See, for example, Table 1. Multiple mechanisms may be responsible for the low retention and limited functional improvements, such as apoptosis of the delivered cells, venous drainage, and the contraction of the beating heart. As short-term cell retention is a prerequisite for long-term cell engraftment and/or functional benefit, methods to attenuate cell loss are highly desirable, yet remain undiscovered to date. According to several embodiments, magnetic targeting represents a non-invasive approach to coax therapeutic agents (e.g., drugs, cells) into desired regions of the heart, in particular the myocardium.

The present study was performed to evaluate myocardial targeting of administered stem cells, long-term cardiac engraftment, and functional recovery of infarcted hearts treated with intramyocardially injected iron-labeled stem cells (e.g., cardiosphere-derived cells; CDCs) subjected to an external magnetic attractor. While the present study employed direct injection of iron-labeled CDCs to the heart and external magnetic attraction, it shall be appreciated that, as described herein, other cell types, target tissues, labeling molecules, and/or attractive forces may be used without departing from the scope of several embodiments described herein.

### Methods

### CDC Culture and SPM Labeling

CDCs were cultured from tissue samples of hearts explanted from 8-week-old male Wistar Kyoto (WKY) rats, as described above. Briefly, rat hearts were excised and biopsies of the left ventricle were cut into 1 to 3 mm³ pieces with a sterile scalpel. The minced tissues were digested with 0.25% trypsin (Invitrogen, Carlsbad, Calif., USA) for 5-10 min and then cultured on fibronectin-coated tissue culture dishes using media consisting of Iscove's Modified Dulbecco's Medium (IMDM, Invitrogen) supplemented with 20% fetal bovine serum (PBS), 100 U/mL penicillin G, 100 μg/mL streptomycin, and 0.1 mmol/L beta-mercaptoethanol. This media is referred to as cardiosphere media (CEM). After 10-20 days, a layer of fibroblast-like adherent cells and a smaller number of phase-bright cells migrated from the tissue explants. The fibroblast-like adherent cells were washed with PBS and detached with TRYPLE™ Select (trypsin, Invitrogen) at room temperature. The harvested cells were then seeded into poly-D-lysine coated 6-well plates (cell density of ˜1×10⁵ cells/well), in CEM containing 10% FBS. Under these suspension culture conditions, the cells self-aggregate into cardiospheres in 3-10 days. Cardiospheres were harvested, seeded in fibronectin-coated tissue culture flasks for expansion as monolayers in CEM containing 20% FBS to generate CDCs, which as described above, express several stem cell markers (e.g., c-kit, CD105, CD90 or CD31). In several embodiments, other culture flasks, treated or not, may be used. In some embodiments, alternative coatings may be used, for example, poly-lysine, extracellular matrix (natural or synthetic), polyethyleneimine polymer, and the like.

CDCs were labeled with fluorescent (dragon green or flash red) superparamagnetic microsphere (SPM) particles (0.9 μm diameter; Bangs Laboratories) as discussed above. Briefly, after 2 passages, rat CDCs were labeled with SPM particles by co-incubation of the cells with SPMs for 24 hr. Labeling efficiency was assessed by flow cytometry, also as discussed above.

### Effects of SPM Labeling on CDC Properties

The SPMs used are a class of superparamagnetic iron oxides (SPIOs). FDA-approved SPIOs are nontoxic, biocompatible and have been used as MRI contrast agents in human subjects. Thus, in several embodiments, the effect of labeling stem cells with SPMs (or other varieties of SPIO) does not substantially impact the properties of the recipient cells nor does it pose adverse health risks to recipients of labeled stem cells.

In vitro toxicity experiments were performed 24 hours after SPM labeling. Cell viability was assessed by Trypan Blue exclusion. Cell proliferation was assessed by seeding approximately 200,000 SPM-labeled and non-labeled cells into 25 cm² (T25) tissue culture flasks. After 2 and 6 days of culture, cells were harvested from the flasks and viable cells were manually counted by Trypan Blue exclusion to determine the proliferative activity of CDCs. For assessment of cell adhesion activity, both SPM-labeled and control cells were seeded at the same initial density onto fibronectin-coated dishes. At 30 min, 2 hours and 4 hours after cell seeding, the media was removed and the flask washed by PBS 3 times to remove floating cells. Attached cells were then harvested, counted and quantified as a percentage of the initial seeding number.

Reactive oxygen species (ROS) formation was detected by confocal imaging using the IMAGE-IT™ LIVE Green Reactive Oxygen Species Detection Kit (Invitrogen). Quantitative ROS measurement was performed by staining cells with 6-carboxy-2′,7′-dichlorodihydrofluorescein diacetate di(acetoxymethyl ester) (Invitrogen) and then measuring fluorescence intensity with a SpectraMax M5 plate reader (Molecular Devices, Sunnyvale, Calif.). Plain CDCs and H₂O₂-treated CDCs were included as negative and positive controls, respectively.

Cell apoptosis and necrosis were assessed by flow cytometry using a LSR11 equipment (BD Biosciences, San Jose, Calif.) and the Annexin V-PE Apoptosis Detection Kit I (BD Pharmingen 559763). Fluorescent compensation was included using single-labeled controls. The percentage of positive cells was defined as the percent of the population falling above the 99th percentile of an isotype control cell population.

Phenotypic characterization of the cells was performed by flow cytometry analysis of the percentage of cells that expressed the antigens c-kit, CD31, CD34 and CD90, The following monoclonal antibodies and conjugated fluorochromes were used with corresponding isotype controls: CD31 (BD Pharmingen 555445), CD34 (Chemicon CBL555F); CD90-FITC (Dianova DIAl20); c-Kit (BD Pharmingen 550412). Data, analysis was performed using flow cytometry software (Flow-Jo 7.2.2 Treestar Inc., Ashland, Oreg.).

### In Vitro Cell Capture Experiments

SPM-labeled CDCs (500:1 SPM:cell ratio) were re-suspended in PBS (approximately 1 million cells/mL) in a 15 mL conical tube. A 1.3 Tesla magnet was applied directly to the outside tube wall or approximately 1 cm away from the tube for 20 seconds. Cell condensation was assessed visually. To better simulate the contracting and turbulent environment of myocardium, the same magnet was mounted on the outside wall of a cell suspension tube which was rotated at 60 RPM. After 24 hours, cell condensation by magnetic capturing was visually examined.

### Cell Injection and Magnetic Targeting

Animal care was in accordance to Institutional Animal Care and Use Committee guidelines. Female WKY rats (Charles River Laboratories, Wilmington, Mass.) (n=88 total) underwent left thoracotomy in the 4th intercostal space under general anesthesia. The heart was exposed and myocardial infarction was produced by permanent ligation of the left anterior descending coronary artery, using a 9-0 silk suture, immediately before cell injection. CDCs (total of approximately 1 million; SPM-labeled or non-labeled; suspended in 100 μl of PBS) were injected directly into the myocardium, at 4 sites into the border zone of infarction (e.g., 250,000 cells in 25 μl PBS per site), using a 29G needle. For magnetic targeting, a 1.3 Tesla circular NdFeB magnet (Edmund Scientifics, Tonawanda, N.Y.) was placed above the heart on the retractor during and 10 min after the cell injection. The chest was closed and animal was allowed to recover after all procedures. In several embodiments, greater or lesser magnetic forces may be used to target the labeled cells (e.g., about 0.2 to 0.5 T, about 0.5 to 0.7 T. about 0.7 T to about 1.0 T, about 1.0 T to about 1.3 T, about 1.3 T to about 1.5 T, and overlapping ranges thereof). In some embodiments, greater or lesser cell numbers are used. For example, in some embodiments, about 250,000 to about 500,000 cells are delivered, from about 500,000 to about 750,000, about 750,000 to about 1 million, from about 1 million to about 2 million, from about 2 million to about 5 million, from about 5 to about 10 million, from about 10 to about 20 million, and overlapping ranges thereof. In several embodiments, the greater efficiency of cell delivery and retention generated by magnetic targeting allows for delivery of a smaller number of cells as compared to non-targeted cells.

Moreover, depending on the cell number (and cell density in the cell suspension to be delivered, less than 4 injection sites are used in some embodiments. However, in some embodiments, for example, those embodiments where a large infarct area would best be treated by a larger number of cells, a larger number of cells can be injected. In some embodiments, 4-5, 5-7, 7-9 or more injection sited are used.

The animals received intramyocardial injections with one of the following randomly-assigned conditions:
Fe-CDC+Magnet group: injection of approximately 1 million SPM-labeled cells in 100 μL PBS with a 1.3 Tesla magnet applied above the apex during the injection and for 10 min after injection;
Fe-CDC group: injection of approximately 1 million SPM-labeled cells in 100 μL PBS without magnet application
CDC group: injection of approximately 1 million non-labeled cells in 100 μL PBS with magnet applied above the apex during the injection and for another 10 min after injection
Control group: injection of 100 μL PBS without cells
SPM control group: injection of 5×10⁸ SPM beads (no cells) in 100 μL PBS with magnet applied.

A camcorder was attached to the surgical microscope to capture videos during cell injection. In some embodiments, other delivery routes are used. For example, in several embodiments, intravenous delivery in conjunction with magnetic targeting is used. In some embodiments, intracoronary delivery in conjunction with magnetic targeting is used.

In several embodiments, the route of administration plays a role in determining the methods by which a magnetic field is applied to (or around) a target tissue. For example, in some embodiments, magnetic fields are applied non-invasively, e.g., externally. For example, in some embodiments, an external magnetic field may be generated by applying a fixed magnet to the chest of a subject. In some embodiments, magnetic fields which are electronically focused and shaped are used.

### Quantification of Engraftment by Real Time PCR

CDCs isolated from male rates were injected into female rats, enabling detection of the SRY gene (located on the Y chromosome) as an index of engraftment. Quantitative PCR was performed 24 hr and 3 weeks after cell injection in 6 animals from each cell-injected group to quantify cell retention/engraftment. The whole heart was harvested, weighed, and homogenized. Genomic DNA was isolated from aliquots of the homogenate corresponding to 12.5 mg of myocardial tissue, using commercial kits (DNA Easy minikit, Qiagen). The TAQMAN® real time PCR assay (Applied Biosystems, CA) was used to quantify the number of transplanted cells with the rat SRY gene as template (forward primer: 5′-GGA GAG AGG CAC AAG TTG GC-3′, reverse primer: 5′-TCC CAG CTG CTT GCT GAT C-3′, TaqMan probe: 6FAM CAA CAG AAT CCC AGC ATG CAG AAT TCA G TAMRA, Applied Biosystems). A standard curve was generated with multiple dilutions of genomic DNA isolated from male hearts to quantify the absolute gene copy numbers. All samples were spiked with equal amounts of female genomic DNA as control. The copy number of the SRY gene at each point of the standard curve is calculated with the amount of DNA in each sample and the mass of the rat genome per cell. For each reaction, 50 ng of template DNA was used. Real time PCR was performed with an Applied Biosystems 7900 HT Fast real-time PCR System. All experiments were performed in triplicate. The number of engrafted cells per heart was quantified by calculating the copy number of SRY gene in the total amount of DNA corresponding to 12.5 mg of myocardium and then extrapolating to the total weight of each heart.

### Fluorescence Imaging (FLI)

CDCs were labeled with SPMs that were conjugated with flash-red fluorophores (excitation: 660 nm; emission: 690 nm), as described above. Due to its long wavelength, flash-red is preferred over dragon-green for imaging purposes in some embodiments (e.g., those embodiments wherein cells are labeled with a reporter gene such as GFP). Representative animals from each cell-injected group were euthanized at 24 hours and 3 weeks after cell injection for fluorescence imaging purposes. The heart, lung and spleen were harvested. Organs were placed in an IVIS 200 imaging system (previously Xenogen Corporation; now Caliper Life Sciences, Mountain View, Calif.) to detect flash-red fluorescence. Extensive PBS wash was performed to remove any cells adherent to the epicardium. Excitation was set at 640 nm and emission was set at 680 nm. Exposure time was set at 5 seconds and maintained the same during each imaging experiment. Fluorescence signals (photon/s) from a fixed region of interest (ROI) were measured and quantified with the Xenogen software. Organs from the CDC group (animals receiving non-labeled CDCs) were used as controls for background noise. Fluorescence signals (photon/s) from a fixed region of interest (ROI) were measured.

### Echocardiography

To assess global cardiac function in 53 rats (Fe-CDC+Magnet [n=12], Fe-CDC [n=12], CDC [n=11], PBS control [n=9] and SPM control [n=9]), echocardiography was performed with the Vevo 770 system (Visual Sonics, Toronto, Canada) on day 0 post-MI and 3 weeks post-MI. The left ventricular ejection fraction (LVEF) was measured from the parasternal long-axis view. LVEF was calculated with Visual Sonics V1.3.8 software from 2D long-axis views taken through the infarcted area. Both absolute values and changes from baseline (day 0 post-MI) were determined.

### Histologic and Morphometric Analysis

Subpopulations of CDCs from each group were virally-transduced to express green fluorescent protein (GFP). In these cases, flash-red-conjugated SPMs were used to avoid crossover with the fluorescence of GFP. Animals receiving GFP cells and flash-red SPMs were sacrificed at 24 hours or 3 weeks after injection.

Hearts were cryo-sectioned and representative slides from each depth range were selected for immunohistochemistry. Sections every 100 μm of the infarct and infarct border zone area (10 μm thickness) were prepared and immunocytochemistry for GFP and CD-68 (macrophages) was performed, using a rabbit anti-GFP (Abeam, Cambridge, Mass., USA) and a Mouse anti rat CD68 (Abeam, Cambridge, Mass., USA) primary antibody respectively. At the 3 week time point, immunocytochemistry for cardiomyocytes and endothelial cells were performed using a mouse anti-alpha-sarcomeric actin (Sigma) and rabbit anti-von Willebrand factor (Abeam) primary antibody respectively. Images were taken by a Leica TCS SP5 X confocal microscopy system.

Quantitative morphometry analysis was performed according to established methods. Briefly, 5-6 animals in each group were euthanized at 3 weeks and the hearts were harvested and frozen in OCT compound. Sections every 100 μm (10 thickness) were prepared. Masson's trichrome staining was performed according to established methods. Images were acquired with a PathScan Enabler IV slide scanner (Advanced Imaging Concepts, Princeton, N.J.). From the Masson's trichrome-stained images, morphometric parameters including L V cavity circumference, total LV circumference, risk region area, scar area, non-infarcted region wall thickness and infarct wall thickness were measured in each section with NIH ImageJ software. To quantify both the degree of LV dilation and the degree of infarct wall thinning, the LV expansion index was calculated as: LV Expansion index=(LV cavity circumference/total LV circumference)×(non-infarcted region wall thickness/risk region wall thickness). The percentage of viable myocardium as a fraction of the risk region was also quantified.

### Statistical Analysis

Results are presented as mean±SD unless specified otherwise. Statistical significance between baseline and 3 week LVEFs was determined using 2-tailed paired Student's t test. All the other comparisons between any 2 groups were performed using 2-tailed unpaired Student's t test. Comparison among more than 2 groups was analyzed by One-Way ANOVA followed by Bonferroni post hoc test. Differences were considered statistically significant when p<0.05.

### Results

### SPM Labeling Minimally Affects Cell Viability and Function

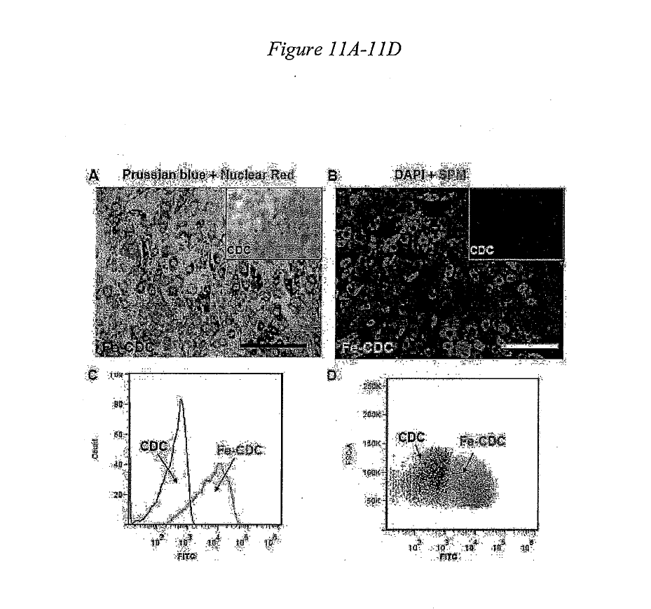

As discussed, CDCs were labeled with Dragon-green fluorescence-conjugated SPM particles by co-incubation in culture for 24 hours. Prussian Blue staining and fluorescence microscopy confirmed particle uptake by CDCs (FIGS. 11A and 11B). Non-labeled cells did not exhibit Prussian Blue or Dragon-green fluorescence (Insets, FIGS. 11A and 11B). These labeled cells are hereafter called SPM-labeled CDCs, or Fe-CDCs for short. Flow cytometry confirmed the labeling of cells with the SPM particles (FIGS. 11C and 11D).

Flow cytometry revealed an average labeling efficiency of 86.4±1.2% when a 500:1 SPM:cell ratio was used. In some embodiments, labeling efficiency is greater, depending on the cells being labeled and the label:cell ratio. In some embodiments, labeling of cells occurs at an efficiency of 75% or more, 80% or more, 85% or more, or 90% or more, including 92, 92, 93, 94, 95, 96, 97, 98, and 99% efficiency. As discussed herein, label:cell ratio may also be varied in some embodiments, including using a ratio of about 250:1, about 500:1, about 750:1, about 1000:1, about 2000:1, or about 4000:1. Cell size, labeling particle size, detection sensitivity and other factors will determine not only the optimal label:cell ratio, but also will impact the efficiency of labeling, in some embodiments.

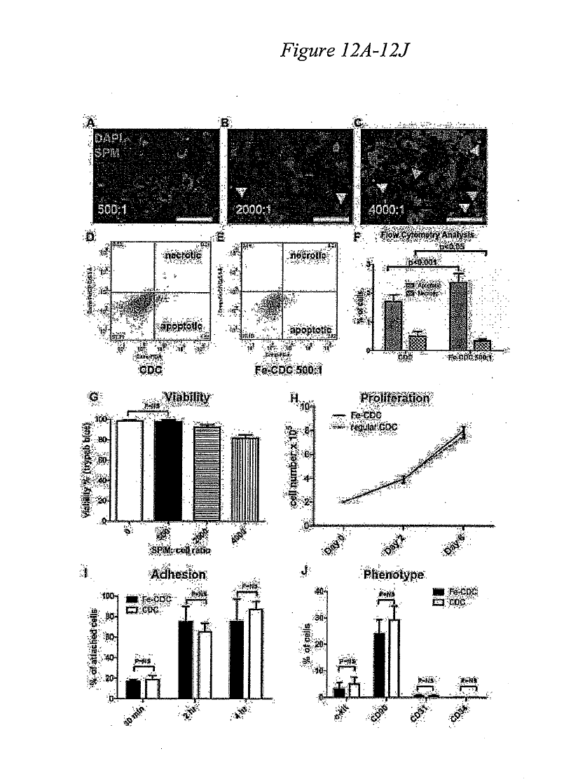

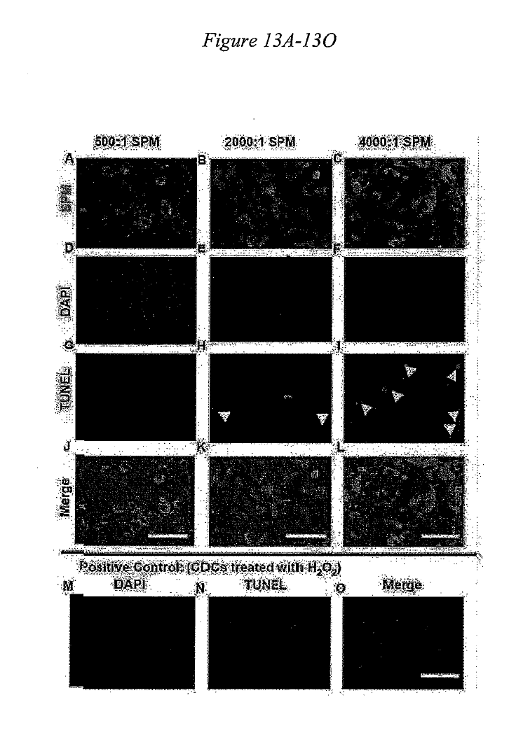

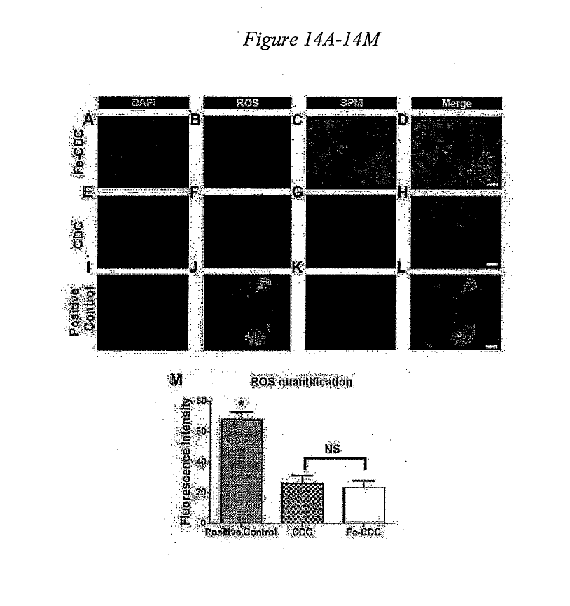

As shown in FIGS. 12 and 13, the number of TUNEL^{POS} apoptotic cells increased with escalating SPM:cell ratio (red cells with white arrowheads FIGS. 12A-C; FIG. 13A-13O). It may be that more SPMs were taken up by each cell at higher SPM dosages. FIGS. 12D and 12E show typical Annexin/7-AAD flow cytometry plots. Further quantification (FIG. 12F) indicated that SPM labeling induced <1% increase of apoptotic cells, but the SPM-labeled group had fewer necrotic cells. Given the fact that 500:1 labeling caused minimal cytotoxicity, this dosage was chosen for subsequent in vitro and in vivo experiments. However, in some embodiments, greater or lesser ratios of label:cells are used. FIGS. 12G-12J show that labeling with SPMs did not affect cell viability, proliferation, adhesion or antigenic phenotype of CDCs. In addition, SPM labeling did not lead to the generation of intracellular reactive oxygen species (ROS), as shown in FIGS. 14A-14M.

Thus in several embodiments, the process of labeling stem cells with a magnetic particle is not significantly adverse to the viability of the cell. In some embodiments, modest increases in apoptosis of cells occur during the labeling process, however, in some such embodiments, necrosis of the cells is lessened. As such, a healthy and viable labeled cell population is produced according to several of the labeling embodiments described herein. Moreover, such procedures, in some embodiments, do not affect the antigenic phenotype or proliferation of the labeled cells. In some cases, this is key, as the delivery of the cells (with their stem-like characteristics) and high degree of proliferation capacity enhances the direct repair of damaged cardiac tissue. Additionally, the ability of the cells, post-labeling, to proliferate within the target tissue, enhances the indirect repair of damaged tissue, in some embodiments.

### External Magnet Captures SPM-Labeled CDCs In Vitro

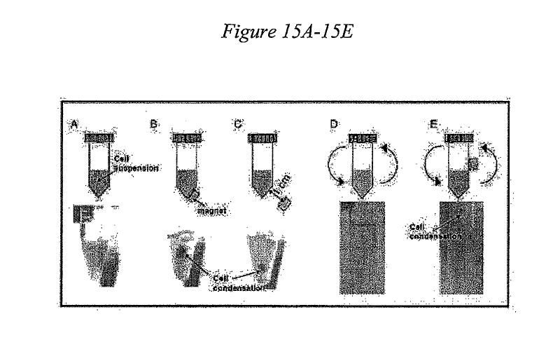

To investigate the ability of a magnet to capture SPM-labeled CDCs in vitro, CDCs were loaded with 500:1 SPMs and re-suspended in a conical tube (See FIG. 15A-15E). After applying the magnet directly on the outer wall of the tube, CDCs were rapidly attracted towards the magnet and accumulated focally on the adjacent inner wall (FIG. 15B). To gauge the effect of a more remote magnetic field, the magnet was moved 1 cm away from the tube and the capture experiment was repeated (FIG. 15C). SPM-labeled CDCs were still rapidly attracted towards the magnet and attached focally, albeit with smaller cell condensates. To better mimic the myocardial environment, where turbulent flow exists, the same magnet was mounted on the outside of a rotating tube containing Fe-CDC suspension. Without the magnet, the cell suspension was uniform, with no focal condensation (FIG. 15D). However, with the external magnet, Fe-CDCs formed a distinct condensate on the inner wall adjacent to the magnet (FIG. 15E, arrow). In some embodiments, magnetic fields are used in the cell preparation process, e.g., to enrich a labeled cell population.

### Magnetic Targeting Captures Fe-CDCs During Injection and Attenuates Washout Effect

Approximately one million CDCs derived from syngeneic male WKY rats were injected intramyocardially into the peri-infarct region of female hearts. White light imaging revealed that the majority of SPM-labeled CDCs (evident from their yellow-brown color) washed out within seconds, diffusing from the injection site towards the base and then quickly disappearing. Such data indicate that initial washout accounts for significant cell loss. In contrast, Fe-CDCs injected with a magnet placed ˜1 cm above the cardiac apex moved towards the apex and accumulated around the infarct (Video data not shown). More cells were visible after injection in a heart from the Fe-CDC+Magnet group than in the Fe-CDC group. Thus, the external magnetic force was capable of effectively opposing the hydraulic forces that ordinarily drive washout.

Thus, in some embodiments, magnetic fields used to target the labeled cells are generated in close proximity to the eventual target tissue. In some embodiments, an invasive technique is used to generate a magnetic field within or surrounding a target tissue. For example, in some embodiments, a cell delivery catheter with a magnetic tip is used to both deliver and target cells to specific areas of a damaged heart. In some embodiments, an electrically (e.g., computer) generated magnetic field is focused from an external position onto an internal target site. In some embodiments, an MRI magnetic field is used. In several embodiments, a simple external magnet may be placed on or near the chest of a subject receiving labeled cells.

### Magnetic Targeting Improves Short-Term Retention and Long-Term Engraftment

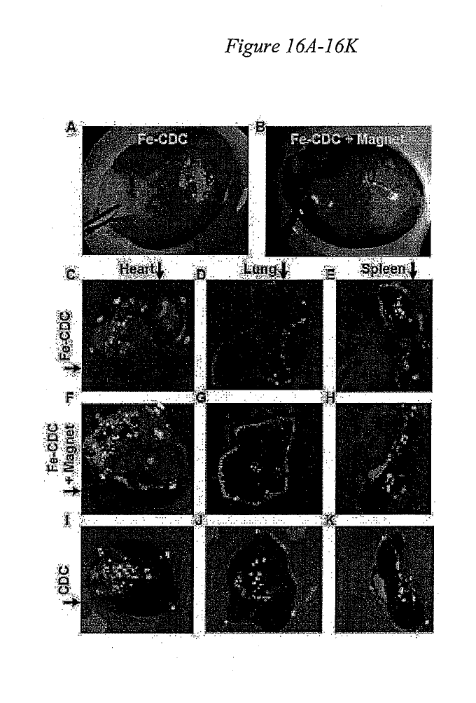

Six animals from each cell-injected group were sacrificed 24 hours after cell injection to assess short-term cell retention. Visual inspection of the excised hearts revealed that the Fe-CDC+Magnet group (FIG. 16B, red arrow right portion of heart) had significantly more cells around the injection area than did the Fe-CDC group (compared with FIG. 16A). Likewise, representative FLI images revealed more flash-red fluorescence in a heart from the Fe-CDC+Magnet group (FIG. 16F) than in the Fe-CDC group (FIG. 16C). To compare off-target migration, lungs and spleens from the same animals were also harvested and imaged. While red fluorescence signals were detectable in the lungs, signal was reduced in the lungs from the Fe-CDC+Magnet group (FIG. 16G) than in those from the Fe-CDC group (FIG. 16D). Thus, the magnet improves the retention of CDCs in the heart. The CDCs, absent the magnetic targeting, would otherwise end up in non-target organs due to venous dispersion. Small amounts of fluorescence were detected in the Fe-CDC spleen (FIG. 16E). This may reflect off-target CDCs, or alternatively, clearance of SPM particles by spleen macrophages. In either case, such fluorescence is markedly reduced in the Fe-CDC+Magnet spleen (FIG. 16H). As a negative control, excised organs from the CDC group (animals injected with non-labeled cells) were also imaged. No fluorescent signals were detectable in any organs (FIGS. 16I-K).

Thus, in several embodiments, the magnetic targeting of cells advantageously retains a higher degree of delivered cells within or around the desired target tissue. Not only does this increase the potential for retention and eventual engraftment of the cells (see below), but it increases the efficiency of the overall treatment regime. For example, the lessened off-target deposition of the labeled cells, in some embodiments allows for a smaller overall number of cells to be delivered (since fewer cells are lost). As a result, the initial tissue size from which cells are isolated may be smaller in some embodiments, which may favor a less invasive collection procedure. In some embodiments, a single harvested piece of donor tissue may give rise to a number of cells that can be used to in a greater number of treatments (e.g., more autologous treatments to the donor, or more cells to be used in a greater number of allogeneic treatments). Moreover, the reduction in off-target cell deposition, in some embodiments, reduces the risk to the recipient of unintended effects, and thereby provides for a safer treatment for the subject.

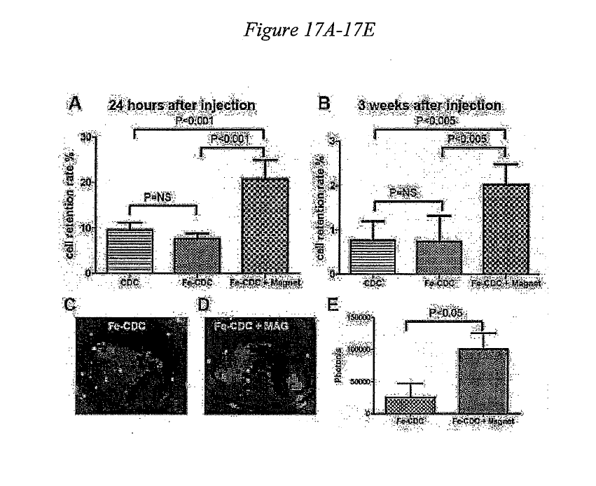

To further assess the numbers of surviving CDCs in the myocardium, quantitative PCR for the male-specific SRY gene was performed. qPCR results confirmed that magnetic targeting enhanced short-term (e.g., about 24 hours post-delivery) cell retention in the recipient hearts: the Fe-CDC+Magnet group exhibited ˜3-fold greater cell numbers than the Fe-CDC group (FIG. 17A). The Fe-CDC+Magnet retained over 20% of the injected cells, which is significantly more than the retention in the Fe-CDC group or the CDC group. Cell retention was indistinguishable in the Fe-CDC group and the CDC group, confirming the lack of an effect of labeling per se on retention. In some embodiments, short term retention is enhanced by at least about 5%. In some embodiments, short term retention is enhanced by about 5% to about 10%, about 10% to about 15%, about 15% to about 20%, about 20% to about 30%, or even about 30% or more, as well as overlapping ranges thereof. In some embodiments, short term retention is enhanced by about 2-fold, about 3-fold, about 4 fold, about 5-fold, about 10-fold, about 20-fold, or greater.

To examine the effect of magnetic targeting on long-term engraftment, subsets of animals in each group were followed for 3 weeks and then sacrificed for qPCR and FLI. PCR results indicated that all three groups experienced a large decrease from the 24 hour time point. (note Y-axis scale on FIG. 17 B as compared to FIG. 17A). However, the Fe-CDC+Magnet group still exhibited enhanced cell engraftment relative to the Fe-CDC group (about 2% retention versus about 0.8% retention, p<0.005; FIG. 17B). Again, SPM labeling itself did not affect engraftment, as the Fe-CDC group was not significantly different from the CDC group. Thus, the equivalence of the CDC and Fe-CDC groups at 24 hours (FIG. 17A) and 3 weeks (FIG. 17B) confirms the idea that SPM labeling does not affect cell proliferation in vivo, assuming the attrition rate of transplanted CDCs is identical in the two groups. FLI images showed more flash-red fluorescence in the hearts from the Fe-CDC+Magnet group (FIG. 17D) than in the Fe-CDC group (FIG. 17C). Quantification of fluorescence intensity revealed a ˜4-fold greater signal in the Fe-CDC+Magnet group (FIG. 16E). These results suggest that magnetic targeting increases both short-term (24 hours) and long-term Fe-CDC engraftment (3 weeks) in the injured myocardium.

In several embodiments, long-term engraftment is enhanced by magnetic targeting of stem cells. As used herein, the term “long term” shall be given it ordinary meaning and shall also refer to any time period greater than about 24 hours (the approximate time for “short term” as used herein). For example, long term may refer to time frames of 28-36 hours, 48 hours, 72 hours, 96 hours, 5-7 days, 2 weeks, one to two months, or several years. In some embodiments, long term engraftment is enhanced (as compared to non-targeted cells) by magnetic targeting by about 5% to about 10%, about 10% to about 15%, about 15% to about 20%, about 20% to about 25%, about 30% to about 35%, about 35% to about 40%, about 40% to about 45%, about 45% to about 50%, and overlapping ranges thereof. In some embodiments, long term engraftment is enhanced by about 50% or more. In some embodiments, long term engraftment is enhanced by about 2-fold, about 3-fold, about 4 fold, about 5-fold, about 10-fold, about 20-fold, or greater. In still additional embodiments, long term retention is approximately equivalent to short term retention (e.g., the enhanced short term retention is maintained). In some embodiments, the absolute value of long term engraftment is less than the absolute value of short term retention, however the enhanced retention as compared to non-targeted cells is maintained.

### Magnetically-Targeted Cell Delivery Attenuates Left Ventricular Remodeling and Enhances the Therapeutic Benefit of Cell Transplantation

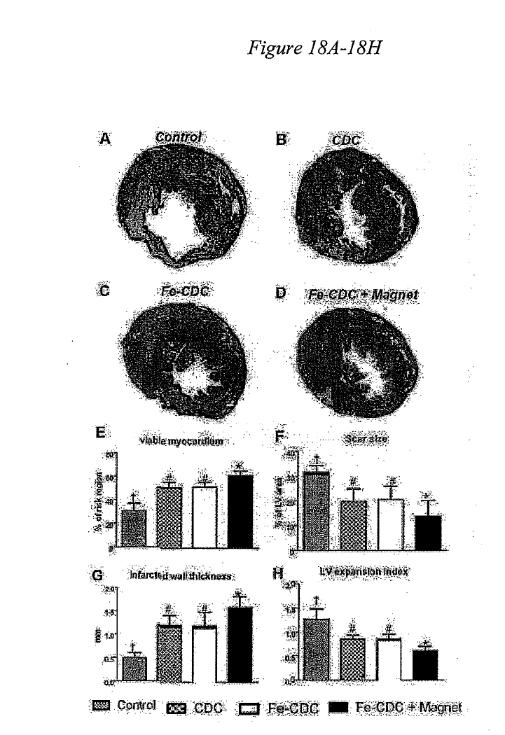

Morphometric analysis of explanted hearts (n=5-6 from each group) at 3 weeks showed severe LV chamber dilatation and infarct wall thinning in PBS-injected hearts (FIG. 18A). In contrast, the three cell treated groups (FIGS. 18B-D) each exhibited attenuated LV remodeling. The protective effect was greatest in the Fe-CDC+Magnet group, which had more viable myocardium (FIG. 18E) and thicker infarcted walls (FIG. 18G), but smaller scars (FIG. 18F) and less LV expansion (FIG. 18H). The Fe-CDC and CDC groups were indistinguishable in these measures, indicative of a similar treatment effect in those two groups. In several embodiments, magnetic targeting of stem cells leads to structural changes in damaged cardiac tissue that are indicative of regeneration and/or recovery of tissue.

In some embodiments, greater quantities of viable myocardium are present after magnetic stem cells are targeted to damaged tissue. In some embodiments, the increase in viable myocardium is due to the delivery of the cells themselves (e.g., the delivered cells have repopulated the damaged tissue). In other embodiments, the increase is due to an indirect effect (e.g. generation of a pro-viability paracrine factor milieu) on the damaged tissue (or the surrounding tissues). In several embodiments, viable myocardium is increased by magnetic targeted cell delivery by about 5% to about 10%, about 10% to about 15%, about 15% to about 20%, about 20% to about 25%, about 30% to about 35%, about 35% to about 40%, about 40% to about 45%, about 45% to about 50%, and overlapping ranges thereof. In some embodiments, viable myocardium is increased by about 2-fold, about 3-fold, about 4 fold, about 5-fold, about 10-fold, about 20-fold, or greater.

In some embodiments, cardiac wall thickness is improved, as compared to an untreated heart and/or to a heart treated with stem cells, but without magnetic targeting. In some embodiments, wall thickness maintenance (or regeneration) is a direct effect of the targeted cells, or in some embodiments, it is due to an indirect effect. It shall also be appreciated that in some embodiments, a combination of direct and indirect effects lead to the improvements in morphometric (or any other) functional measure. In several embodiments, wall thickness is improved (as compared to non-targeted treated hearts or compared to untreated hearts) by 5% to about 10%, about 10% to about 15%, about 15% to about 20%, about 20% to about 25%, about 30% to about 35%, about 35% to about 40%, about 40% to about 45%, about 45% to about 50%, and overlapping ranges thereof. In some embodiments, improvements in wall thickness can also range from about 50% to about 75%, about 75% to about 100%, about 100% to about 150%, and overlapping ranges thereof. Depending on the severity of damage to a heart, magnetic cell targeting can, in some embodiments, improve wall thickness by greater than 150%. In some embodiments, wall thickness is increased by about 2-fold, about 3-fold, about 4 fold, about 5-fold, about 10-fold, about 20-fold, or greater.

In conjunction with improved wall thickness and viable myocardium, in some embodiments scar tissue is reduced. Scar tissue results from the death of cardiac tissue due to an injury such as an infarct, and its rigidity and minimized contractility can compromise cardiac function. Thus, in some embodiments, targeting of magnetic cells reduces the amount of scar tissue, which in turn improves post-infarct cardiac function, by about 5% to about 10%, about 10% to about 15%, about 15% to about 20%, about 20% to about 25%, about 30% to about 35%, about 35% to about 40%, about 40% to about 45%, about 45% to about 50%, and overlapping ranges thereof. In some embodiments, scar tissue is reduced by about 2-fold, about 3-fold, about 4 fold, about 5-fold, about 10-fold, about 20-fold, or greater.

In several embodiments, the expansion of the left ventricle is reduced by the magnetic targeting of stem cells to an injured heart. As a result, such treated tissue reveals a left ventricular expansion index that is greater than non-targeted cell therapies or control treatments. In some embodiments, the expansion index is improved by 5% to about 10%, about 10% to about 20%, about 20% to about 40%, about 40% to about 80%, about 80% to about 100%, and overlapping ranges thereof. In some embodiments, improvements in the expansion index exceed 100%. In some embodiments, the left ventricular expansion index is improved by about 2-fold, about 3-fold, about 4 fold, about 5-fold, about 10-fold, about 20-fold, or greater.

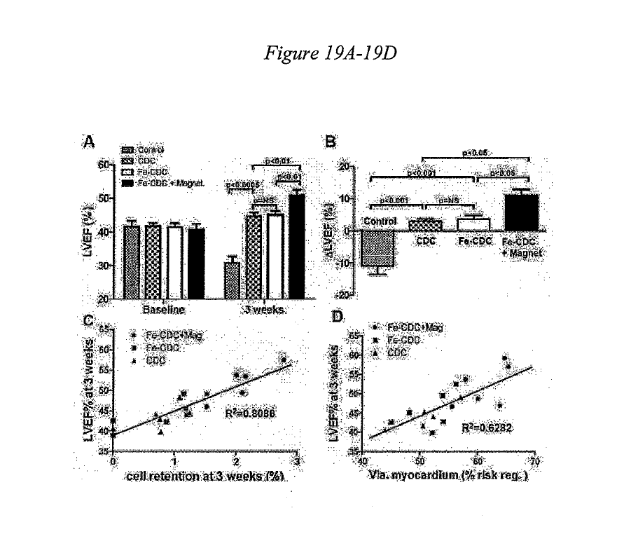

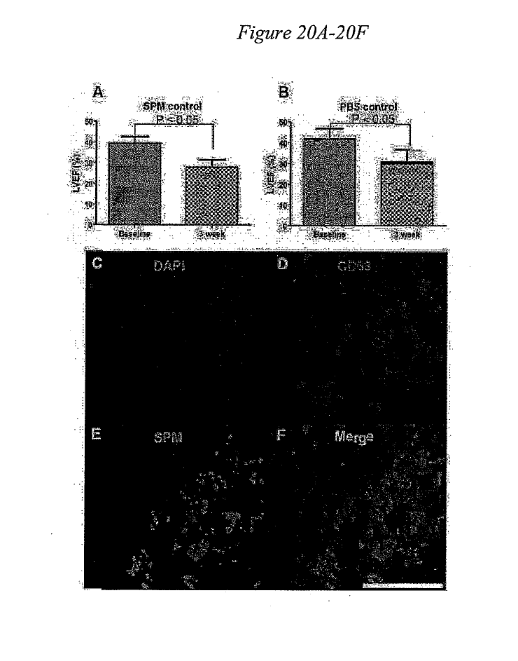

To investigate whether improved cell retention/engraftment translates to enhanced functional benefit, global LVEF was assessed by echocardiography at baseline (Day 0 after MI and cell injection) and 3 weeks later. LVEF at baseline did not differ between treatment groups, indicating a comparable degree of initial injury (FIG. 19A). Over the three weeks after infarction, LVEF declined progressively in the control group (PBS-injected animals) (FIG. 19A), while LVEF improved in all three groups receiving CDCs. These results confirm that cardiac function can be significantly improved by transplantation of CDCs. Notably, however, the Fe-CDC+Magnet group exhibited significantly better cardiac function compared to either the Fe-CDC group or the CDC group (FIG. 19A, p<0.01). The LVEFs in the Fe-CDC and CDC groups were indistinguishable, again demonstrating that SPM loading did not undermine the salutary effects of CDCs. To facilitate comparisons among groups, the treatment effect, the change in LVEF at 3 weeks relative to baseline, was determined in each group (FIG. 19B). PBS injection had a negative treatment effect, as the LVEF decreased over time. In contrast, the Fe-CDC+Magnet group exhibited a sizable positive treatment effect (−12% increase), which is significantly greater than that in either the Fe-CDC or CDC groups (approximately 3 and 4%, respectively). The treatment effect in the Fe-CDC group was no different than that in the CDC group. In addition, injection of SPMs alone or PBS (no cells) had no beneficial effects (FIG. 20A-20B).

In some embodiments, magnetic targeting of stem cells improves cardiac function by a clinically significant margin. In some embodiments, this is represented by increases in LVEF of about 2.5% to about 5%, from about 5% to about 7.5%, from about 7.5% to about 10%, from about 10% to about 15%, from about 15% to about 20%, and overlapping ranges thereof. In some embodiments, improvements of greater than 20% may be realized. In some embodiments, LVEF is increased by about 2-fold, about 3-fold, about 4 fold, about 5-fold, about 10-fold, about 20-fold, or greater.

To further investigate the relationships between long-term cell retention or myocardial viability on one hand, and cardiac function on the other, 3-week LVEFs were plotted individually against percentages of engraftment (FIG. 19C) or viable myocardium in the risk region (FIG. 19D) at 3 weeks. Improved heart function was clearly associated with higher cell retention rate (R2=0.8086; FIG. 19C) and increased myocardial viability (R2=6282; FIG. 19D) by linear regression analysis. These composite functional results indicate that the improved cell retention and engraftment in the Fe-CDC+Magnet group translated into superior functional benefit and attenuation of LV remodeling. Thus, in several embodiments, improved cell retention is correlated with improved cardiac function and/or structural remodeling of the heart that leads to improved function. In some embodiments, mathematical correlation is not discovered, however functional improvement results despite a smaller than expected degree of cell retention or engraftment. For example, targeting of magnetic CDCs may, in some embodiments, result in a lower than expected degree of engraftment, however, in such embodiments, it is possible that indirect (e.g., paracrine effect) mechanisms still produce an improvement in function or structure.

Magnetic Targeting Enhances Cell Engraftment and does not Adversely Impact Inflammation

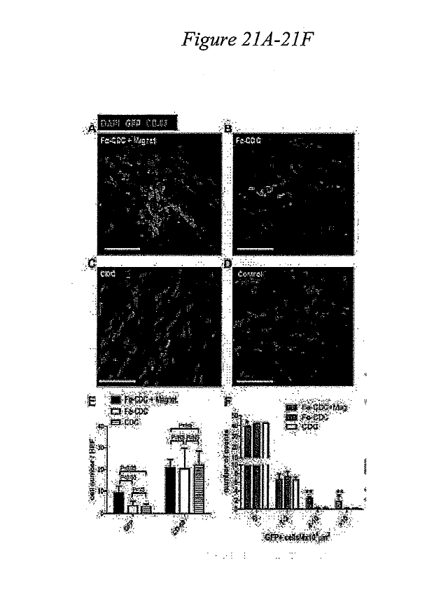

To further characterize engraftment, hearts from representative animals in each group were harvested 3 weeks after injection and cryo-sectioned for immunohistochemistry. Confocal imaging enabled the detection of transplanted cells (GFP; green); macrophages (CD68; red); and all cell nuclei (DAPI; blue). FIG. 21 shows representative confocal images (21A: Fe-CDC+magnet; 21B: Fe-CDC; 21C: CDC; 21D: Control). Cell numbers were quantified as positive cells per high power field (HPF; FIG. 21E) and reveal significantly more GFP-positive cells in the Fe-CDC+Magnet group compared to the Fe-CDC or CDC groups. These data are consistent with the PCR results discussed above showing greater long-term cell engraftment with magnetic targeting. In some experiments, GFP-positive multi-cellular clusters were frequently observed in the Fe-CDC+magnet group (FIG. 21A).

To quantify the effects of magnetic targeting on the spatial distribution of transplanted cells, GFP-positive cells from 50 randomly selected fields (4×10⁴ μm²) were counted and the number of events was plotted against varying cell numbers (FIG. 21F). Most of the fields examined were devoid of transplanted cells in the control, CDC, and Fe-CDC groups. However, the Fe-CDC+Magnet group had more engraftment area (less “empty” area) compared to the Fe-CDC or CDC group (p<0.05). The number of fields with 1-3 engrafted cells was indistinguishable among all the three groups. Interestingly, the Fe-CDC+Magnet group had many more fields with 4-10 or >10 engrafted cells than the Fe-CDC or CDC group (p<0.001). Thus, it appears that magnetic targeting increases engraftment in focally-condensed patches rather than homogeneously. However, in some embodiments, a more homogeneous distribution of stem cells may be detected. In several embodiments, however, the focally-condensed patches still result in a more global recovery effect, as demonstrated by the functional and morphometric recovery data discussed above. Thus, some embodiments of the present invention do not require an even distribution of cells across the damaged tissue area in order to effect repair of damaged tissue. In some embodiments, pockets of magnetically targeted cells are sufficient because the indirect repair mechanisms (e.g., paracrine effects) are active in a penumbra surrounding each pocket. Thus while the delivered cells exist in focally-condensed patches, the beneficial effects are more far-reaching than the pocket, even overlapping in some embodiments, thereby effecting a more widespread repair or regeneration of damaged tissue.

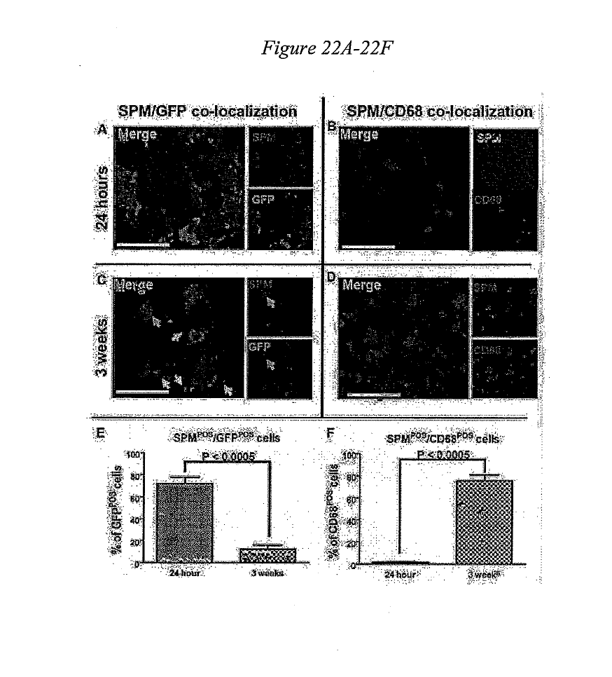

One potential concern regarding SPMs and magnetic targeting is the possibility of an inflammatory response. However, to assess inflammation, CD-68^{POS} macrophages were quantified. The tissue density of CD-68^{POS} macrophages was comparable in all three groups. These observations indicate that the presence of SPMs in the host tissue did not cause or worsen inflammation. Notably, at the 3 week time point the majority of GFP-positive cells are SPM-negative; only ˜10% of transplanted cells still contained SPMs (FIG. 22E). A shift of SPMs from the transplanted CDCs to resident macrophages was clearly evident when sections were compared at 24 hours versus 3 weeks (FIG. 22F). These observations suggest that Fe-CDCs expel SPMs via exocytosis in vivo, followed by endocytosis by macrophages and eventual incorporation into body's iron stores. Thus, in some embodiments, despite the presence of a foreign or non-self particle being introduced into the heart, inflammatory responses are minimal as compared to unlabeled CDCs. In several embodiments, the limited immune response is advantageous, as the infiltration of macrophages into infarcted tissue is coordinately reduced. Thus, in some embodiments, macrophages, or other immune responsive cells, are not preferentially targeting labeled CDCs. In some embodiments, the magnetic particles function solely to aid in the delivery of the stem cells to the target tissue. For example, the evaluation of engraftment and macrophage infiltration in the short term reveals, in some embodiments, a high degree of labeled cells in the target myocardium. In some embodiments, the number of delivered cells that have retained their labeled particles approaches 80%. In some embodiments, greater or lesser labeling may be detected at short term time points. In some embodiments, the passage of time results in a significant reduction of the number of cells that retain the label. In some embodiments, the passage of several weeks time reduces the percentage of labeled cells by 20%, 30%, 40%, 50%, or more. However, as demonstrated by the functional and morphometric data discussed herein, repair and/or recovery of damaged tissue is not contingent on the long term presence of the label on the cells, or necessarily the long term retention of the cells themselves.

In some embodiments, the iron particles used to label the delivered cells are simply reabsorbed over time, resulting in modest (if any) increases in iron levels. In some embodiments, no morbidity or mortality results from the iron particles, as the potential for chemical toxicity of parenteral iron is known and appreciated in the art. In several embodiments, iron from injected SPMs will eventually be incorporated into the body's own iron stores. The typical total amount of iron oxide used for diagnostic imaging purposed (40-200 mg Fe) is small compared to the total human iron stores (around 3500 mg). In some embodiments, the amount of iron used for magnetically-targeted cell therapy is roughly equivalent to the exposure from diagnostic imaging techniques. In some embodiments, the iron exposure is significantly less. For example, in the present CADUCEUS phase I clinical trial (see http://www.clinicaltrials.org), an estimated maximum of 25 million CDCs are administered to each study subject. As a non-limiting example calculation, based on the fact that every SPM particle contains approximately, 0.5 pg of iron oxides, use of a 500:1 SPM to cell ratio with 25 million cells would yield only 6.25 mg of iron administered to each patient, which represents less than 0.2% of normal iron stores.

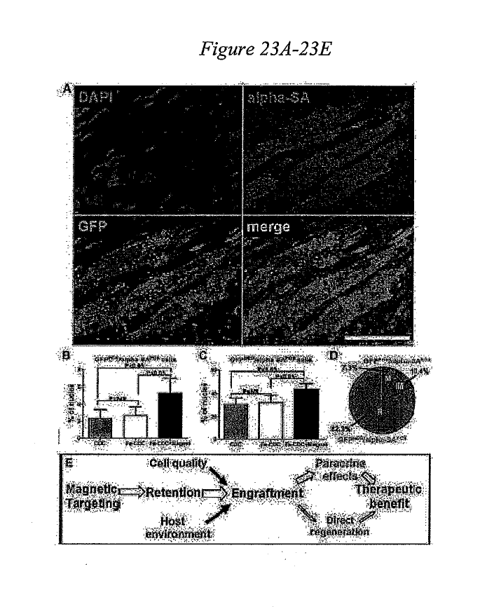

In several embodiments, stem cell delivery to a damaged or diseased heart improves cardiac function by direct regeneration (e.g., differentiation of delivered cells into cardiac tissue), while in some embodiments, indirect mechanisms (e.g., paracrine stimulation of regenerative cascades) are responsible. In still additional embodiments both direct and indirect mechanisms of cardiac regeneration/repair are simultaneously (or sequentially) involved. To assess whether transplanted Fe-CDCs differentiate into cardiac cells (a direct repair mechanism), tissue sections from each treatment group were stained for cardiac α-sarcomeric actin; alpha-SA) and endothelial (von Willebrand factor; vWF) markers. GFP^{POS}/alpha-SA^{POS} cells were consistently detected (FIG. 23A, merged panel). Co-expression of these markers indicates that transplanted cells have differentiated into cardiomyocytes, as the CDCs do not express alpha-SA prior to delivery. The Fe-CDC+Magnet group had significantly more GFP^{POS}/alpha-SA^{POS} and GFP^{NEG}/alpha-SA^{POS} cells than the CDC or Fe-CDC group (FIG. 23B). The greater degree of GFP^{POS}/alpha-SA^{POS} cells attests to the creation of new myocardium by direct differentiation. As depicted in FIG. 23C, the significant increase in GFP^{NEG}/alpha-SA^{POS} cells reflects an increase in indirect mechanisms (recruitment of endogenous regeneration mechanism or cells and/or tissue preservation) of cardiac repair. Indirect mechanisms (GFP^{NEG}/alpha-SA^{POS}) contributed to ˜83% of the regeneration (see FIG. 23D). Thus in several embodiments, delivered stem cells function to directly replace the damaged, diseased, or dead cells in the target tissue. In some embodiments, the differentiation of the delivered stem cells into cardiac phenotypes allows for the direct replacement of damaged tissue by the delivered cells. In some embodiments, one or more cardiac-specific markers that were not present on the CDCs at the time of delivery are present on the cells within the tissue post-delivery. For example, alpha SA, cardiac troponin and the like are increased in some embodiments.

In some embodiments, however, the presence of new cells in the damaged cardiac tissue is not directly due to the presence of delivered cells, as demonstrated by the presence of GFPNEG cells that are alpha-SAPOS (representing cells that were not exogenously delivered). Thus in several embodiments, indirect recruitment of cells to the damaged area (from remote areas of the heart) is involved in the repair of the damaged tissue. In some embodiments, local and/or paracrine effects result in recruitment of such cells, and or increase the viability of the cells within or closely surrounding the damaged area.

To further dissect the mechanism of benefit of magnetic targeting, the magnet-related increment in various cell populations: recipient-derived myocytes, mature donor-derived myocytes and immature donor-derived myocytes was calculated (FIG. 23D). Binucleation was used to distinguish between mature and immature myocytes; as binucleated myocytes were distinctly longer than mononucleated myocytes, with a typical length:width ratio >3:1. Direct regeneration (GFPPOS/alpha-SAPOS cells) contributed 17.7% of the total benefit; of that percentage, an absolute 7.3% was comprised of mature donor-derived myocytes (e.g., the transplanted cells). In relative terms, 41.2% of the total donor-derived myocytes were binucleated.

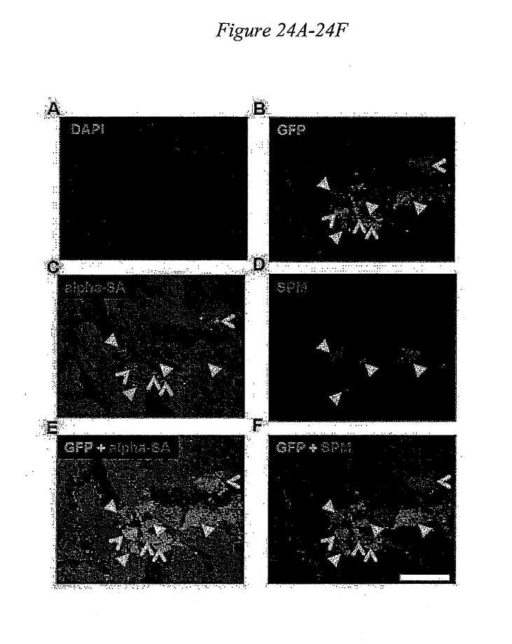

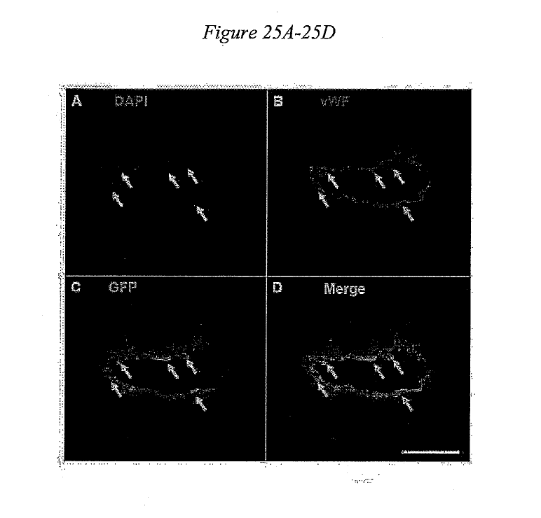

These quantitative data also indicate that SPM labeling has a negligible impact on in vivo cardiac differentiation, as the CDC and Fe-CDC groups had similar densities of GFP^{POS}/alpha-SA^{POS} cells (FIG. 23B). In addition, remnant SPMs in the cytoplasm did not prevent Fe-CDCs from differentiating into a cardiomyocyte phenotype, as SPM/GFP/alpha-SA triple positive cells were detected (FIG. 24 panels B-F; highlighted with white solid arrowheads). Endothelial differentiation was also confirmed by the presence of GFP^{POS}/vWF^{POS} cells (FIG. 25A-25D, white arrows). Thus, in several embodiments, delivered cells have the capacity to differentiate into one or more of the types of tissue that are important for generating new cardiac tissue (e.g., cardiomyocytes, smooth muscle cells, and endothelial cells).

Taken together, these results suggest that, in several embodiments, transient magnetic targeting (e.g., exposure to an external magnetic field for about 10 minutes) has a “butterfly effect” on subsequent cell therapy outcomes in that both functional benefits (e.g., FIG. 19) and long-term cell engraftment (e.g., FIG. 21) are realized.

In several embodiments, magnetic targeting improves short-term cell retention which subsequently boosts long-term engraftment. In some embodiments, enhanced long-term engraftment translates into greater therapeutic benefit by both indirect (paracrine) and direct regeneration mechanisms. A schematic representing a possible cascade of events taking place in several embodiments, is shown in FIG. 23E. In some embodiments, increased short-term retention due to magnetic targeting occurs in the absence of increased long-term cell retention, yet therapeutic benefits are still realized. In such embodiments, the short-term retention initiates the cascade of events that leads to indirect regeneration mechanisms. Thus, in some embodiments, short-term engraftment is sufficient to yield positive therapeutic effects. As demonstrated and discussed above, in some embodiments, indirect mechanisms play the primary role in regeneration of cardiac tissue. However, in some embodiments, direct and indirect mechanisms play equivalent roles. In still additional embodiments, direct regeneration plays a larger role than indirect regeneration. In some embodiments, the increased role of direct regeneration is associated with even more pronounced increases in short- and/or long-term retention of delivered and targeted cells.

As shown in FIGS. 23A and 23F, a portion of the CDCs surviving at 3 weeks appear in multicellular clusters in the Fe-CDC+Magnet group. In some embodiments, such cellular clusters result from a condensation effect of magnetic targeting. Moreover, in some embodiments, such clusters result in increased regeneration of cardiac tissue, as three-dimensional multi-cellular clusters are generally more resistant to hostile cellular environments, such as the infarcted myocardium. In some embodiments, cell clusters provide mechanical and paracrine support to transplanted neighbors.

### Example 17

### Magnetic Targeting Enhances Cell Retention, Engraftment and Functional Benefit After Intracoronary Delivery of CDCs

As discussed above, the success of cell therapy relies on effective delivery of cells into the desired region of a target tissue. This presents particular challenges in the heart, where venous washout potentiated by cardiac contraction results in substantial cell loss during and immediately after delivery. Successful delivery is particularly challenging when cells are delivered via the intracoronary (i.c.) route, where retention of non-targeted cells is generally quite low due to venous flow. However, the safety, reproducibility and ready translation to clinical application make the i.c. route attractive. The present study was directed to assessing the improvement in i.c. delivery of CDCs due to magnetic targeting.

In many clinical scenarios, acute myocardial infarction (AMI) patients usually first undergo reperfusion, thus magnetic enhancement for i.c. CDC delivery was investigated in a rat model of acute ischemia/reperfusion.

### Methods

### Study Design

A dose-ranging study in a group of non-infarcted animals was conducted to determine the optimal cell dose for i.c. delivery of CDCs. Optimal doses were judged to be those that maximized cell retention without causing micro-embolic injury. After establishing the optimal i.c. dose, that dose was further used to evaluate the efficacy of magnetic enhancement. Three treatment groups were included:
Group 1—I.C. infusion of vehicle (PBS) only (control)
Group 2—I.C. infusion of iron-labeled CDCs without a magnet (Fe-CDC)
Group 3—I.C. infusion of iron-labeled CDCs with a magnet placed above the left ventricle (LV) during and after infusion. (Fe-CDC+magnet group)

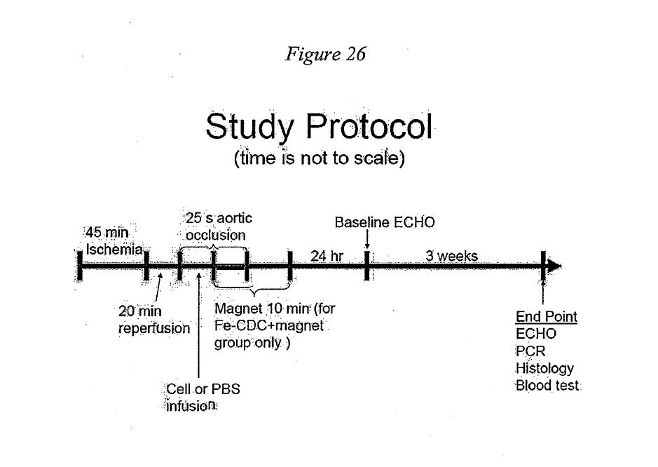

A non-labeled CDC group was not included because no effects of iron loading per se have been found in other studies (see e.g., Example 16). To increase relevance to clinical situations (e.g., reperfused AMI), a coronary occlusion/reperfusion model was used. The general study design is depicted in FIG. 26.

### Rat CDC Culture and SPM Labeling

Rat CDCs were generated and maintained as described above.

### In Vitro Toxicity Analysis

Proliferation of SPM-loaded and control CDCs was assessed with a commercial Cell Counting Kit-8 (CCK-8; Dojindo Molecular Technologies INC, Rockville, Md.). The original manufacturer's instructions were followed. Briefly, cells were seeded at an initial seeding density of 2000 cells per well of a 96-well plate. At pre-determined time points, the CCK-8 reagent was added into representative wells and the plate was incubated at 37° C. for 1 hour. Then, absorbance was measured by a SpectraMax M5 plate reader (Molecular Devices, Sunnyvale, Calif.). For western blot analysis of apoptosis marker Caspase-3, the equivalent total protein from plain and iron-labeled CDCs was loaded onto SDS-PAGE gels, and then transferred to PVDF membranes. After overnight blocking in 3% milk TBS-T, membranes were incubated with 1:1000 mouse anti-Caspase-3 antibody, 1:1000 and 1:3000 dilution of rabbit anti-beta-actin monoclonal antibody (Lifespan Bioscience, Seattle, Wash.), respectively. The appropriate horseradish peroxidase-conjugated secondary antibodies were used, and then the blots were visualized by using SuperSignal West Femto maximum sensitivity substrate (Thermo Scientific) and exposed to Gel Doc™ XR System (Bio-Rad Lab INC). Cell apoptosis was also assessed by TUNEL staining (In Situ Cell Death Detection Kit, TMR red, Roche, Germany). Cell nuclei were counter-stained with DAPI. It shall be appreciated that other embodiments may employ other laboratory techniques to evaluate the toxicity of iron-labeled CDCs.

### Animal Model

Animal care was in accordance to Institutional Animal Care and Use Committee guidelines. Female WKY rats (Charles River Laboratories, Wilmington, Mass.) (n=82 total) underwent left thoracotomy in the 4th intercostal space under general anesthesia. The heart was exposed and myocardial infarction was produced by 45 minute ligation of the left anterior descending coronary artery, using a 7-0 silk suture. Thereafter, the suture was released in order to allow coronary reperfusion. Twenty minutes later, cells from one of the groups above were injected into the left ventricle cavity during a 25 second temporary aorta occlusion with a looped suture. For magnetic targeting, a 1.3 Tesla circular NdFeB magnet (Edmund Scientifics, Tonawanda, N.Y.) was placed above the heart during and after the cell injection. The chest was closed and animal was allowed to recover after all procedures. For dose-ranging study (above), no myocardial infarction was created and the cells were injected with the same protocol.

### Fluorescence Imaging

Fluorescence imaging was performed as described above (see, e.g., Example 16).

### Quantification of Engraftment by Real Time PCR

Quantitative PCR was performed 24 hr and 3 weeks after cell injection in 5 animals from each cell-injected group to quantify cell retention/engraftment. PCR analysis was performed as described above (see, e.g., Example 16).

### Morphometric Heart Analysis

For morphometric analysis, 7 animals in each group were euthanized at 3 weeks (after cardiac function assessment). Morphometric analysis was performed as described above (see, e.g., Example 16).

### Histology

Histological analysis was also performed as described above (see, e.g., Example 16).

### ELISA for Cardiac Troponin I, Transferring and Ferritin

All assays were run according to manufacturer's protocol (Rat Cardiac Troponin-I ELISA, Life Diagnostics, cat no 2010-2-HS; Rat Transferrin and Rat Ferritin, Immunology Consultants Laboratory Inc, cat no E-25TX and E-25F). Serum samples were assayed undiluted for the cTnI ELISA. Serum was diluted 1:40,000 for Transferrin and 1:40 for Ferritin analysis using the provided sample buffer. The absorbance was measured at 450 nm at the assay endpoint and the values of all analytes were initially calculated in nanograms per milliliter.

### Results

Labeling of Rat CDCs with SPMs

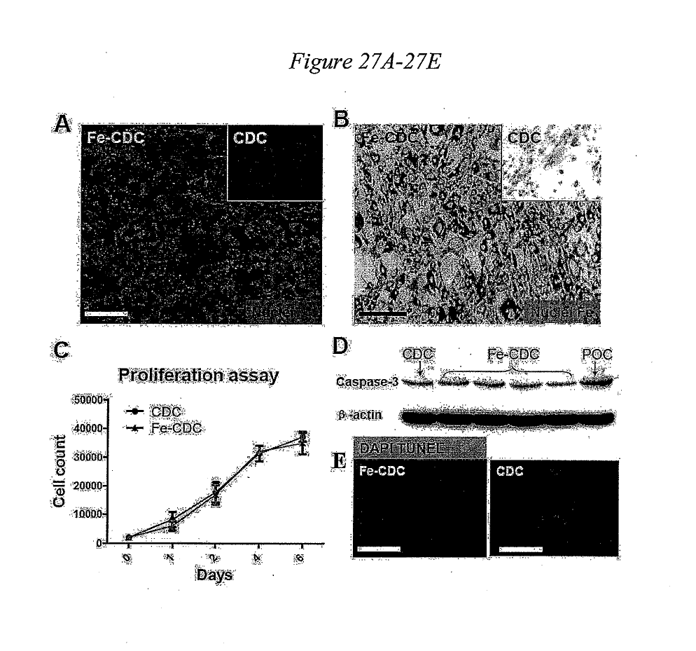

As described above, rat CDCs were labeled with flash red fluorescence-conjugated SPM particles at a ratio of 500:1 (SPMs:cells) by spontaneous endocytosis. Fluorescent microscopy and Prussian Blue staining confirmed particle uptake (FIGS. 27A and 27B). Non-labeled cells did not exhibit flash red fluorescence or Prussian Blue staining (insets, FIGS. 27A and 27B). Labeled cells are hereafter called Fe-CDCs for brevity. The proliferation rates of Fe-CDCs and CDCs were indistinguishable (FIG. 27C). Western blot analysis of Caspase-3 expression and TUNEL staining confirmed that iron labeling did not induce apoptosis in Fe-CDCs (FIGS. 27 D and E, respectively). These findings reconfirm those discussed in Example 16 above, that SPM particles, loaded as described, have minimal toxicity on cells.

### Dose-Ranging Study for Magnetically-Targeted Intracoronary Cell Delivery

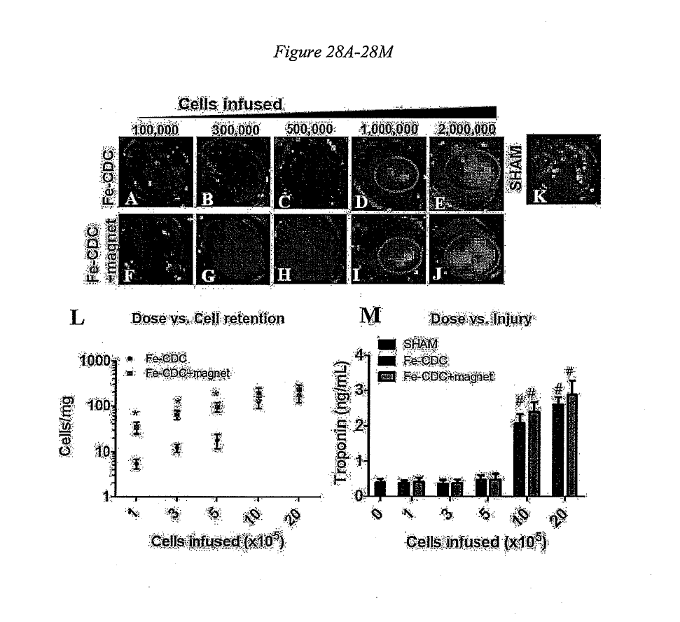

In the dose-ranging study, animals that received magnetically-targeted and non-targeted i.c. Fe-CDCs were sacrificed at 24 hours for assessment of cell retention and myocardial injury. In excised hearts, fluorescence imaging revealed that flash red intensity increased with escalating cell doses (FIGS. 28A-28J) in both cell-infused groups (Panels A-E are Fe-CDCs without targeting, Panels F-J are Fe-CDCs with magnetic targeting). Hearts from the control group (PBS infusion; Panel K) showed no detectable epifluorescence. More cells were evident in the Fe-CDC+magnet group than the Fe-CDC group over a range of low infused doses (1, 3, and 5×10⁵ cells) (see Panel L). At higher doses (1 and 2×10⁶ cells), however, epifluorescence was comparable in the Fe-CDC and Fe-CDC+magnet groups. High-intensity regions were detected in both groups (circled with pink), indicating robust cell retention in those zones.

Microvascular plugging is one potential side effect of cell dosages that are higher than optimal. To maximize cell retention without inducing myocardial damage, cell retention was (measured by real-time PCR) was correlated with serum troponin-I (measured by sTnI ELISA to yield dose/retention and dose/injury relationships (FIG. 28M). Consistent with the fluorescent imaging results, the numbers of cells retained increased with escalating infused cell doses (FIG. 28B). At the three lowest doses, magnetic targeting enhanced cell retention by 5.2-6.4 fold (p<0.05). At the two highest doses, cell retention was equivalent in the Fe-CDC and Fe-CDC+magnet groups (p=0.14 and p=0.15, respectively). sTnI levels from both cell treatment groups at the three low doses were comparable to those from Control infusions, but sTnI was increased in both groups when 1 or 2×10⁶ cells were infused (p<0.001 vs. Control).

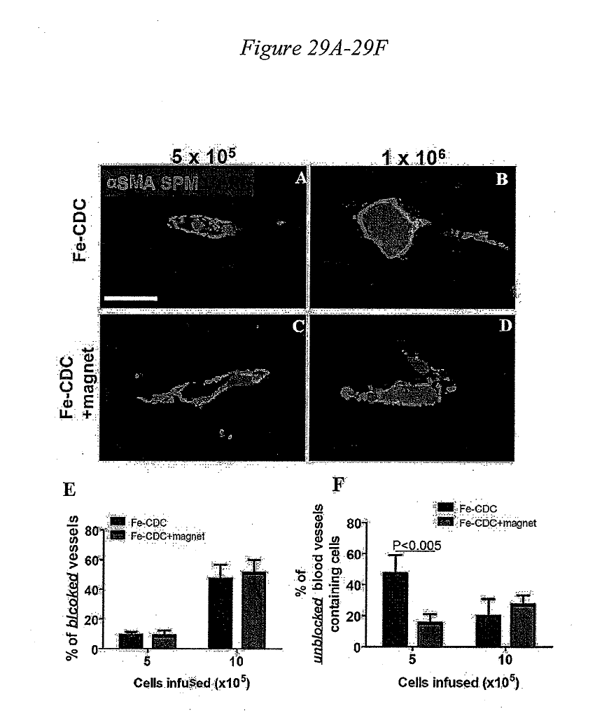

To verify that the elevation of sTnI is due to microembolization, representative hearts were cryo-sectioned and analyzed for co-visualization of alpha smooth muscle actin (αSMA)-positive blood vessels and Fe-CDCs (flash red fluorescence). No microemboli were detected at the dose of 5×10⁵ cells in either group (FIGS. 29A and 29C). Fe-CDCs (magenta) were readily detected within the blood vessels (green), but the vessels were still patent. At the dose of 1×10⁶ cells, clear evidence of embolism was seen as many blood vessels were completely occluded by cell clumps (FIGS. 29B and 29D). The percentage of blocked vessels (FIG. 29E) increased dramatically when the dose was increased from 5×10⁵ to 1×10⁶, in correlation with the increases in sTnI as cell number increased. Again, there was no difference between the Fe-CDC and Fe-CDC+magnet groups, indicating that magnetic targeting itself does not induce or worsen embolic injury. Interestingly, the Fe-CDC+magnet group has more unblocked cell-containing blood vessels (FIG. 29F; P<0.005), consistent with the observed increase in cell retention without sTnI elevation. Based on these results, the largest cell infusion dose without micro-embolic injury (5×10⁵ cells) was utilized in subsequent efficacy studies. However, it shall be appreciated that in some embodiments, other doses, including those doses that showed signs of adverse effects in the present study may be used. As discussed above, other factors come into play in other embodiments, including but not limited to, patient age, general condition, and immunological status. In some embodiments, labeled CDCs are administered in a dose between about 1×10⁴ to about 1×10¹°, between about 1×10⁵ and 1×10⁹, 1×10⁶ and 1×10⁸, such as between 1×10⁷ and 5×10⁷, or overlapping ranges thereof. Depending on the size of the damaged region of the heart, more or less cells can be used. A larger region of damage may require a larger dose of cells, and a small region of damage may require a smaller does of cells. On the basis of body weight of the recipient, an effective dose may be between 1×10⁵ and 1×10⁷ per kg of body weight, such as between 1×10⁶ and 5×10⁶ cells per kg of body weight. In some embodiments, microvascular plugging is circumvented (partially or completely) by delivery of the same total dose of cells, but dividing the delivery into multiple administrations. Also, in some embodiments, wherein larger animals (e.g., humans) are receiving cells, the microvasculature is less likely to become plugged with labeled CDCs.

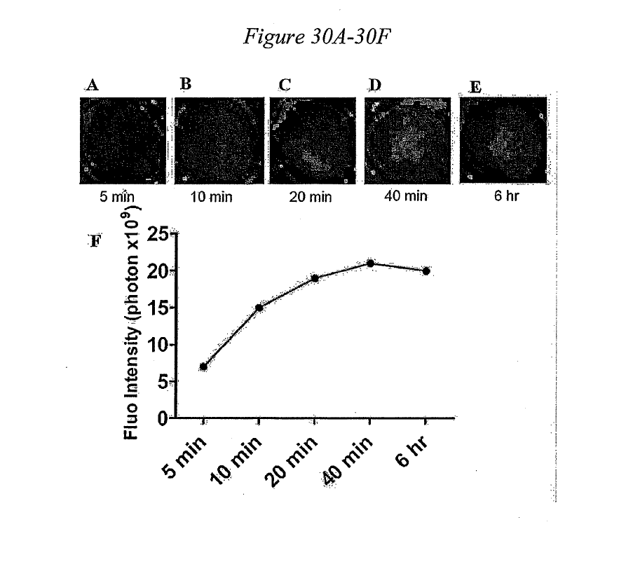

Time of magnet application is another parameter that can be optimized. As in Example 16, 10 min of magnet application induced increases in cell retention and improvements in downstream therapeutic outcomes. In animals receiving 5×10⁵ Fe-CDCs, magnets were applied for either 5, 10, 20, 40 minutes or 6 hours (n=1 for each time point). For the “6 hour” animal, the magnet was mounted outside the chest for 5 hour 20 min after the open-chest 40 minutes magnet application. Twenty-four hours after cell infusion, animals were sacrificed and the hearts were excised for fluorescent imaging (FIGS. 30A-30E). Cell retention increased with duration of magnet application, but only modest increases were detected beyond 10 minutes exposure (see e.g., FIG. 30F). Given that animals become more vulnerable the longer the open-chest interval, 10 minutes was chosen as the magnet application duration for the following efficacy study. As with cell dose, in some embodiments, a longer magnet exposure time is used, while in some embodiments, a shorted magnet exposure time is used. In some embodiments, the magnetic field is applied for about 1 minute up to about 5 hours. In some embodiments, the magnetic field is applied for about 1 minute to 5 minutes, about 5 minutes to about 10 minutes, about 10 minutes to about 20 minutes, about 20 minutes to about 30 minutes, and overlapping ranges thereof. In several embodiments, the magnetic field is applied for about 5-15 minutes, including about 6, 7, 8, 9, 10, 11, 12, 13, or 14 minutes. The strength of the magnetic field is also a factor in determining exposure time. For example, a higher magnetic field strength may applied for less time with equivalent overall cell retention. Likewise, the depth of the damaged tissue is a factor to consider. Stronger magnetic fields and/or longer exposure times are used in some embodiments wherein the damaged cardiac tissue is deep relative to the site of administration. In some embodiments, an entirely external magnet is used, and therefore no concerns arise regarding the vulnerability of the patient to open-chest exposures. In some embodiments, the incremental increase in cell retention seen with longer magnet application times is clinically significant.

### Magnetically-Enhanced Intracoronary Delivery Short-Term Retention and Long-Term Engraftment

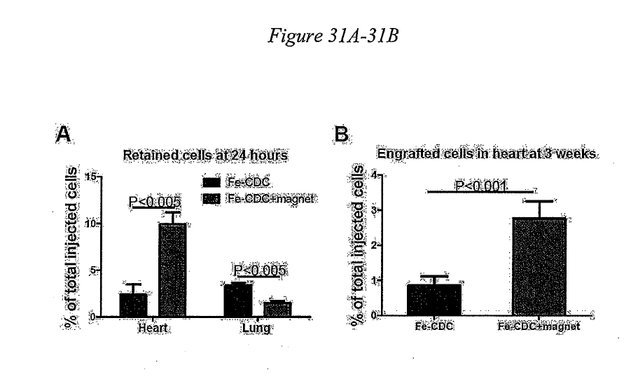

To assess the numbers of surviving CDCs in the myocardium and off-target migration into other organs, quantitative PCR for the male-specific SRY gene was performed 24 hours after cell infusion. Cell retention/engraftment was calculated as the total number of cells detected in the heart divided by the number of infused cells (5×10⁵). Magnetic targeting enhanced short-term cell retention in the recipient hearts as exemplified by the increased cell retention rates in the Fe-CDC-magnet group as compared to the Fe-CDC group (increased cell retention of >4-fold greater; see FIG. 31A; p<0.005). Moreover, fewer cells were detected in lungs from the Fe-CDC+magnet group (FIG. 31A; p<0.005), indicating decreased cell washout into the pulmonary bed. No cells were detected in livers or spleens in either group. The total cell retention at 24 hours (heart+lung) was <15% in both groups, with cell death likely being the major culprit for the reduction in cell number.

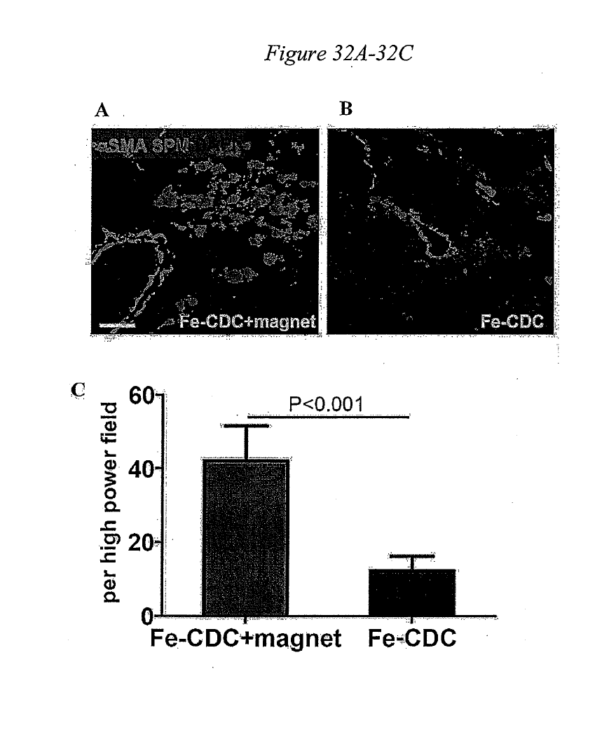

Because vascularly-delivered cells are believed to translocate into the parenchyma at 48-72 hours, a subpopulation of animals was studied histologically at 72 hours. Cells were found residing in the myocardium adjacent to blood vessels in both groups (see e.g., FIGS. 32A and 32B). Greater numbers of myocardium-resident cells were detected in the Fe-CDC+magnet group as compared to the Fe-CDC group (FIG. 32C, p<0.001). Thus, the boost of cell retention at 24 hours resulted in a larger number of cells that eventually migrated across blood vessel walls.

To examine the effects of magnetic targeting on long-term engraftment, subsets of animals from both groups were studied at 3 weeks. Quantitative PCR revealed that both groups experienced a substantial drop (from 24 hours) in surviving cells. However, the Fe-CDC-magnet group exhibited enhanced cell engraftment relative to the Fe-CDC group (FIG. 31B). Thus, in several embodiments, magnetic targeting increases both short-term (24 hours) and long-term Fe-CDC engraftment (3 weeks) in the ischemia/reperfusion-injured myocardium. In some embodiments, short-term retention alone is sufficient to effect long-term physiological benefits. In some embodiments, the short term retention alone induces clinically (e.g., therapeutically significant results). In some embodiments, long-term physiologic benefits are detected and/or realized even in the absence of increased short-term retention.

### Magnetically-Enhanced Intracoronary Delivery of CDCs Attenuates Left Ventricular Remodeling and Enhances the Functional Benefit of Cell Therapy

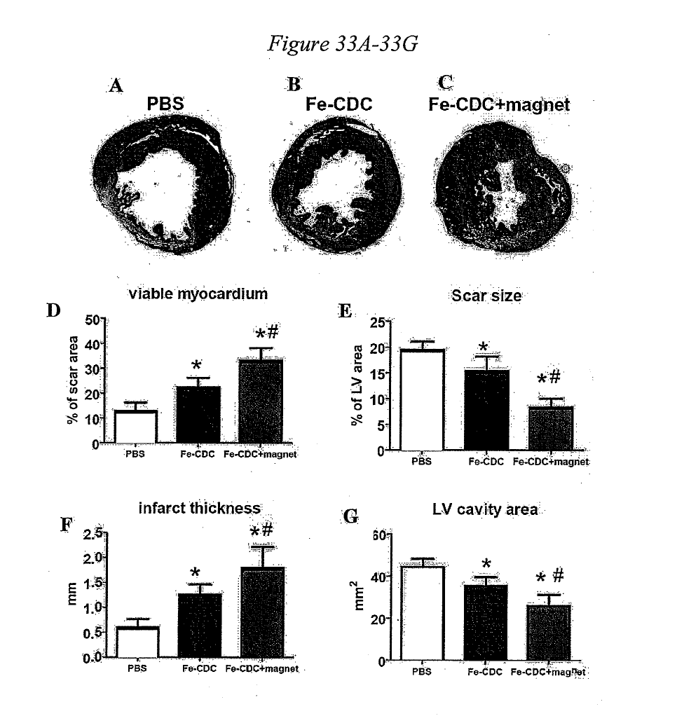

Morphometry at 3 weeks showed severe left ventricular chamber dilatation and infarct wall thinning in the control (PBS-infused) hearts (FIG. 33A). In contrast, the two cell-treated groups exhibited attenuated LV remodeling and improved heart morphology. The protective remodeling effect was greatest in the Fe-CDC+magnet group (FIG. 33C), which had more viable myocardium in the risk region (FIG. 33D) and thicker infarcted walls (FIG. 33F). Likewise, the Fe-CDC+magnet group had smaller scar sizes (FIG. 33E) and smaller LV cavity areas (FIG. 33G) than the Fe-CDC group. In several embodiments, magnetic targeting of stem cells leads to structural changes in damaged cardiac tissue that are indicative of regeneration and/or recovery of tissue. In several embodiments, one or more of viable myocardium, reduced scar size, increased wall thickness, and smaller left ventricular cavity areas are realized after magnetically targeted CDC delivery. In some embodiments, improvement one of the above morphometric parameters is sufficient to yield clinically relevant improvements in cardiac function. However, in several embodiments more than one parameter is improved based on the magnetic targeting of intracoronary CDCs.

In some embodiments, the improvements in structural changes in the myocardium are due to the delivery of the cells themselves (e.g., the delivered cells have repopulated the damaged tissue). In other embodiments, the increase is due to an indirect effect (e.g. generation of a pro-viability paracrine factor milieu) on the damaged tissue (or the surrounding tissues). In several embodiments, structural improvements comprise increases in viable myocardium.

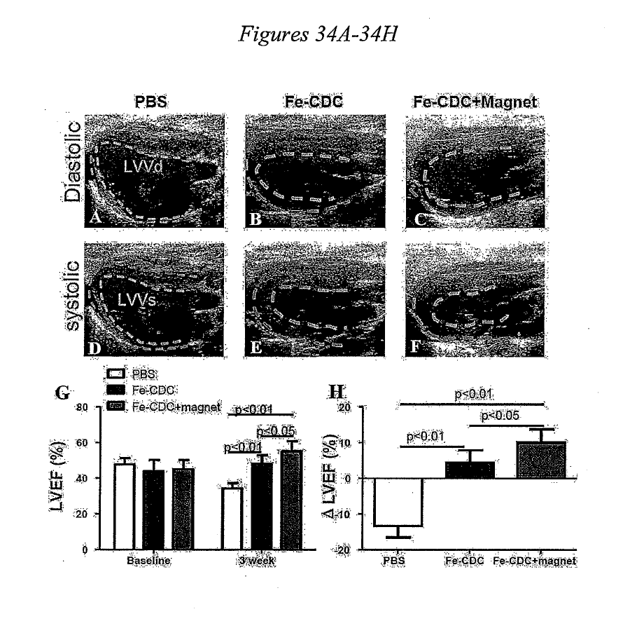

To investigate whether improved cell retention translated to better functional outcomes, left ventricle ejection fraction (LVEF) was assessed by echocardiography at baseline (24 hours after I/R and treatment) and 3 weeks later. FIGS. 34A-34F show representative long-axis diastolic and systolic images at 3 weeks. LVEFs at baseline did not differ between treatment groups, indicating a comparable degree of initial injury (FIG. 34G). Over the next 3 weeks, LVEF declined progressively in the control group, but not in the Fe-CDC treated animals. Notably, the Fe-CDC+magnet group exhibited better therapeutic outcome, with LVEF superior to the Fe-CDC group (p<0.05) at 3 weeks. To facilitate comparisons, the treatment effect, i.e., the change in LVEF at 3 weeks relative to baseline, in each group was calculated (FIG. 34H). Controls had a negative treatment effect, as LVEF decreased over time. In contrast, the Fe-CDC+magnet group exhibited a sizable positive treatment effect, which was even greater than that in the Fe-CDC group (p<0.05). Taken together, these data indicate that an increase in cell retention/engraftment does translate into better heart morphology and greater functional benefit.

Thus, in several embodiments, improved functional characteristics of the damaged myocardium are associated with increases in one or more of cell retention or cell engraftment. In some embodiments, improved function comprises increased cardiac output. In some embodiments, the increase in cardiac output is typified by an increased LVEF. In some embodiments, LVEF is increased by a statistically significant amount. In some embodiments, LVEF is increased by about 5% to about 10%, about 10% to about 15%, about 15% to about 20%, about 20% to about 25%, about 30% to about 35%, about 35% to about 40%, about 40% to about 45%, about 45% to about 50%, and overlapping ranges thereof. In some embodiments, viable myocardium is increased by about 2-fold, about 3-fold, about 4 fold, about 5-fold, about 10-fold, about 20-fold, or greater.

### The Benefit of Magnetic Targeting is Due to Both Direct and Indirect Mechanisms

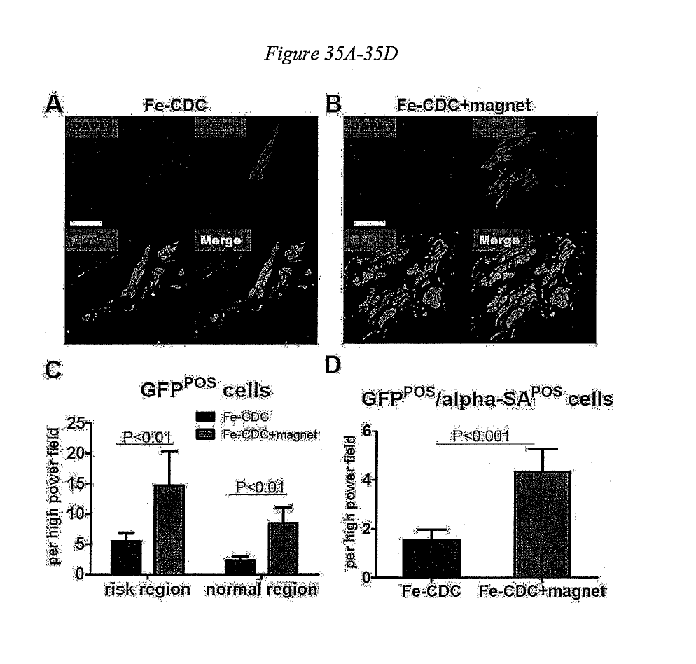

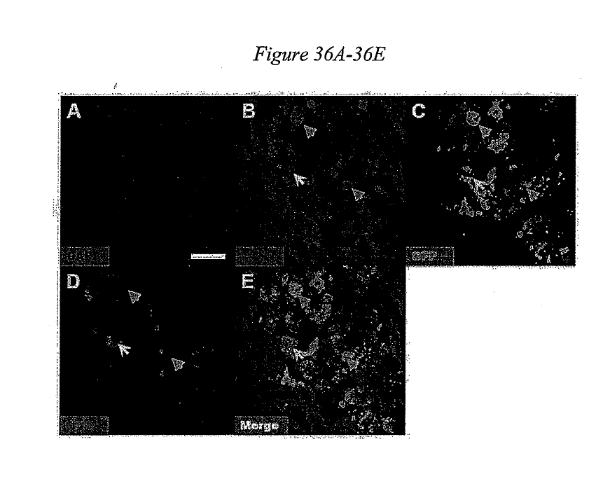

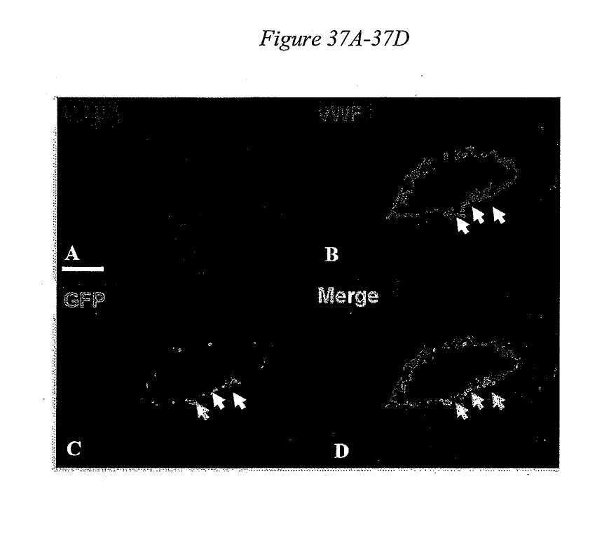

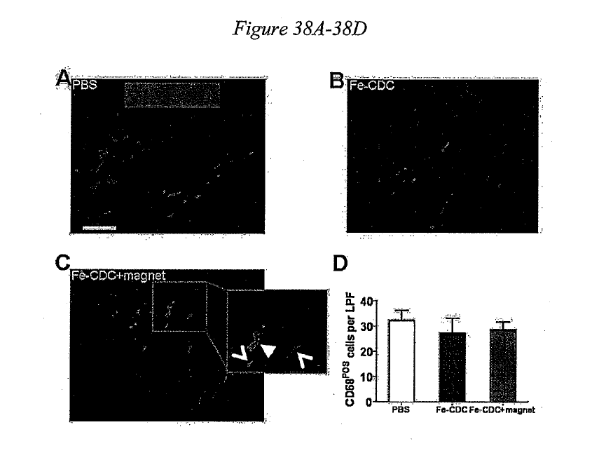

CDCs have been shown to improve cardiac function both by direct regeneration and by indirect mechanisms. To further dissect the mechanism of the extra functional benefit brought about by magnetic targeting, histology was performed at 3 weeks. In this study, Fe-CDCs expressing green fluorescent protein (GFP) by lentiviral transduction were used (rather than iron) to track stem cell fate, as SPM particles left over from cell death or exocytosis can create false-positive signals for “engraftment”. To assess the engraftment and phenotypic fate of transplanted Fe-CDCs, sections of cardiac tissue were stained for GFP (transplanted CDCs or their progeny) and α-sarcomeric actin (cardiomyocytes). Consistent with the PCR results at 3 weeks, more GFP-positive cells were evident in the Fe-CDC+magnet group than the Fe-CDC group, in both risk and normal regions (FIG. 35C; p<0.01). GFP^{POS}/α-sarcomeric actin^{POS} cells, taken to be cardiomyocytes that differentiated from delivered CDCs, were consistently detected (FIGS. 35A and 35B). The Fe-CDC+magnet group had more GFP^{POS}/α-sarcomeric actin^{POS} cells than the Fe-CDC group (FIG. 35D; p<0.001), indicating more cardiomyocytes resulting from direct differentiation. Fluorescent images confirmed that remnant SPMs in the cytoplasm did not prevent Fe-CDCs from differentiating into a cardiomyocyte phenotype, as SPM^{POS}/GFP^{POS}/α-sarcomeric actin^{POS} cells were detected (FIG. 36B/D; white arrows). Endothelial differentiation was also confirmed by the presence of GFP^{POS}/von Willebrand^{POS} cells (FIG. 37; white arrows). Notably, at the 3 week time point the majority of GFP-positive cells were SPM-negative, consistent with the concept that Fe-CDCs expel SPMs via exocytosis, followed by clearance of SPMs by macrophages. CD68^{POS}/SPM^{POS} and CD68^{POS}/SPM^{NEG} macrophages were consistently detected in both the Fe-CDC and Fe-CDC+magnet groups (FIGS. 38B and C). However, the overall tissue density of CD68^{POS} macrophages was similar in all three groups including controls (FIG. 38D), further verifying that iron labeling and/or magnetic targeting did not induce or worsen inflammation in the injured heart. Thus, advantageously, in several embodiments the administration of CDCs containing or carrying a foreign particle used to target the CDCs does not induce a significant immune response. In some embodiments, this is particularly relevant as the infiltration of immune cells may interfere with the direct or indirect mechanisms of cardiac repair. As such, the magnetic targeting of CDCs unexpectedly circumvents significant immune responses.

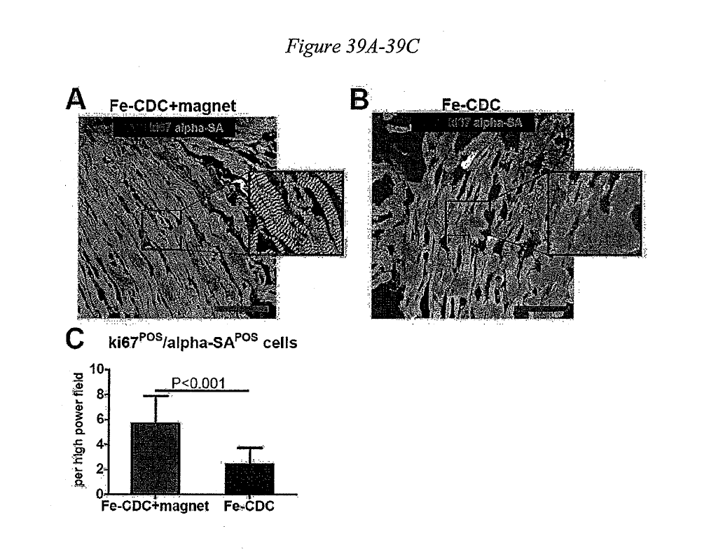

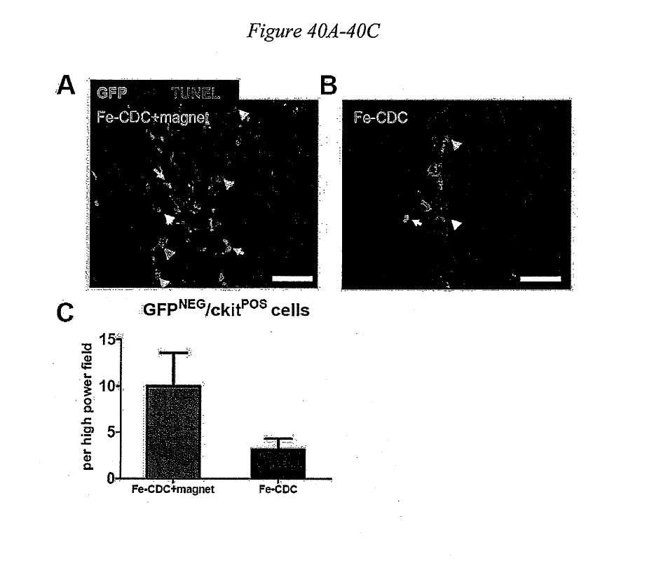

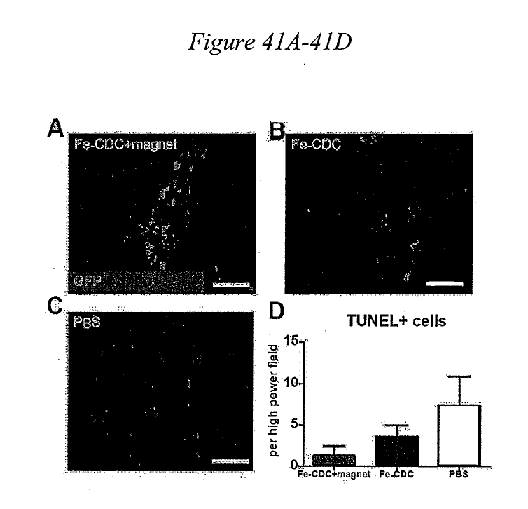

While direct regeneration was consistently detected, the absolute number of GFP+ cardiomyocytes was relatively low given the degree of functional improvement detected. As discussed above, transplanted CDCs, in some embodiments, exert their regenerative potential largely by indirect mechanisms (or paracrine effects). In several embodiments, CDCs produce relatively high levels of various types of pro-angiogenic and anti-apoptotic factors including, but not limited to, VEGF, IGF, SDF-1, HGF, PDGF, bFGF. Those factors support endogenous repair by various mechanisms such as promoting cell cycle re-entry of mature cardiomyocytes, recruiting endogenous stem cells from inside and outside of the heart, and preserving myocardium after ischemic injury. To assess indirect contributions (e.g., paracrine effects) to cardiac repair, heart sections were stained 3 weeks after treatment and quantified ki67^{POS}/alpha-SA^{POS} (proliferating or newly-formed cardiomyocytes), c-kit^{POS}/GFP^{NEG} (endogenous c-kit+ cells), and TUNEL^{POS} (apoptotic) cells were quantified. FIG. 39 shows that more ki67^{POS}/alpha-SA^{POS} cells (green arrows in Panels A and B) were detected in the Fe-CDC+magnet group (p<0.001). Panel C shows the overall quantification of Ki67^{POS}/αSA^{POS} in the two groups. In addition, greater numbers of endogenous c-kit^{POS} cells (FIG. 40; arrowheads) and fewer TUNEL^{POS} cells (FIG. 41) were found in hearts from the Fe-CDC+magnet group. These findings reveal that the benefit of the magnetically-targeted enhancement of CDC engraftment was due to a combination of endogenous recruitment, tissue preservation, as well as direct differentiation of transplanted cells.

Thus, in several embodiments, both direct and indirect mechanisms are responsible for the repair and/or regeneration of damaged cardiac tissue when cell therapy is administered. In some embodiments, when cells are administered via an intracoronary route, direct differentiation of delivered cells and regeneration of new cardiac tissue results. In some embodiments, the administered cells function to recruit cells from other regions of the heart to the site of damage, thereby effecting repair of the damaged tissue. In some embodiments, the administration of cells yields a cascade of signals that preserve either the endogenous tissue, the delivered cells, or combinations thereof. It shall be appreciated, as discussed above, that other routes of administration of cell therapy may also yield such effects.

### Iron Labeling and Magnetic Targeting Induces No Marginal Inflammation or Iron Toxicity

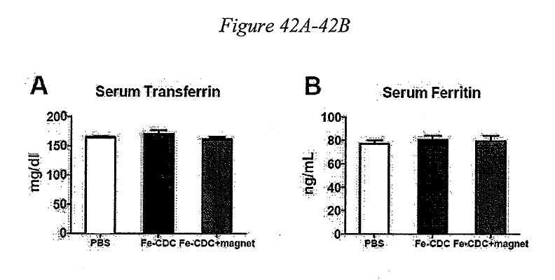

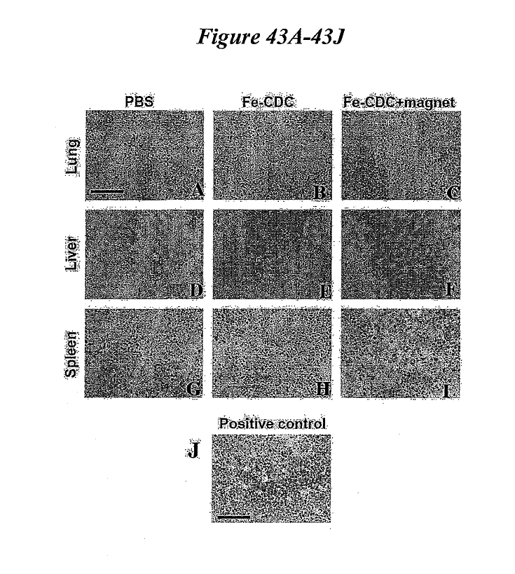

Of concern with any interventional therapy is morbidity and/or mortality attributable to the therapy. For all three groups examined in the study, mortality rates post-AMI were zero. Three weeks after treatment, major organs were examined at necropsy and tumor formation was undetectable. To assess possible iron overload caused by Fe-CDCs, serum ferritin and transferrin levels were measured at 3 weeks. Serum levels were not significantly different as compared to controls for both Fe-CDC-treated groups (FIGS. 42A-42B). Also, Prussian Blue staining did not detect any iron clusters in lungs, livers, or spleens (FIGS. 43C, 43F, and 43I, respectively). Thus, in several embodiments, delivery and magnetic targeting of SPM-labeled cells via an intracoronary delivery route minimizes off target deposition of cells. As a result, in some embodiments, cell deposition outside of the target site is minimized. In several embodiments, intracoronary cell delivery, in conjunction with magnetic targeting, provides a safe and effective means of administering cellular therapy for repair of damaged myocardium.

While there exists a great need for treatments for ischemic heart disease, and while cell therapy is theoretically a viable choice, cyclical cardiac contraction in conjunction with venous washout of delivered cells has, to date, undermined the efficient delivery of therapeutic cells. Intracoronary infusion is a potentially popular route of cell delivery in the clinical setting, especially after AMI, but its effectiveness may be restricted by extremely low cell retention after delivery. Intracoronary delivery typically yields lower cell retention in the heart than intramyocardial (i.m.) injection. Additionally, a greater loss of cells into the pulmonary circulation can limit the effectiveness of therapy as well as increase chances of adverse off-target effects. In some cases, intracoronary delivery standard intracoronary delivery proves less efficacious than direct i.m. administration of therapy.

However, in several embodiments magnetic targeting increases both the safety and viability of intracoronary delivery routes. In some embodiments, I.C. delivery increases short-term cell retention and long-term engraftment, in conjunction with limited off-target deposition, which yields improved therapeutic benefit. In several embodiments, increased short-term cell retention translates into a higher degree of engraftment. In several embodiments, increased short-term cell retention translates into improved heart morphology. In several embodiments, increased short-term cell retention translates into increased functional benefit at 3 weeks. In some embodiments, combinations of all of the above result from intracoronary delivery and magnetic targeting of CDCs.

In several embodiments, the therapeutic benefit of magnetically targeted cells is a result of one or more of direct regeneration (e.g., differentiation of delivered cells) and other indirect mechanisms (e.g., paracrine recruitment of other endogenous cardiac cells and/or paracrine preservation/rescue of cells). In some embodiments, magnetic targeting prevents cells from being washed away during the transient infusion period. In some embodiments, magnetic targeting enhances cell adhesion to increase the chance of transvascular relocation of the delivered cells. In several embodiments, CDC administration via an intracoronary delivery route results in little or no incremental inflammation or iron toxicity. In several other embodiments, alternative delivery routes yield similarly safe and efficacious results.

The embodiments provided herein described above are intended to be merely exemplary, and those skilled in the art will recognize, or be able to ascertain using no more than routine experimentation, numerous equivalents to the specific procedures described herein. All such equivalents are considered to be within the scope of the present invention and are covered by the following claims. Furthermore, as used in this specification and claims, the singular forms “a,” “an” and “the” include plural forms unless the content clearly dictates otherwise. Thus, for example, reference to “a vascular permeability agent” includes a mixture of two or more such agents. Additionally, ordinarily skilled artisans will recognize that operational sequence must be set forth in some specific order for the purpose of explanation and claiming, but the present invention contemplates various changes beyond such specific order.

The contents of all references described herein are hereby incorporated by reference.

1.-98. (canceled)

99. A method for repairing damaged cardiac tissue in a patient comprising:
applying a magnetic field to a region of damaged cardiac tissue in a patient having damaged cardiac tissue,
wherein the application of the magnetic field targets to the region of damaged cardiac tissue a population of stem cells in the patient that have been magnetically labeled to form magnetically labeled stem cells,
wherein the magnetically labeled stem cells comprise stem cells labeled with magnetic particles linked to a first antibody portion and a second antibody portion,
wherein the first antibody portion is bound to a cell surface molecule on the stem cell and the second antibody portion is capable of binding to a cardiac tissue marker expressed in damaged cardiac tissue,
wherein the magnetic field and the second antibody portion enhance the retention and engraftment of the magnetically labeled stem cells to the region of damaged cardiac tissue; and
wherein the enhanced retention and engraftment of the magnetically labeled stem cells in the region of damaged cardiac tissue results in improved cardiac function, thereby repairing said damaged cardiac tissue.

100. The method of claim 99, wherein the stem cells are bound to the magnetic label ex vivo.

101. The method of claim 99, wherein the stem cells are bound to the magnetic label in vivo.

102. The method of claim 99, wherein the first antibody portion and the second antibody portion are contained in a single bi-functional antibody.

103. The method of claim 99, wherein the first antibody portion is contained on a first antibody and the second antibody portion is contained on a second antibody.

104. The method of claim 99, wherein the magnetic field is applied while the patient's heart is actively contracting,
wherein the active contraction induces an efflux of the magnetically labeled stem cells away from the site of damaged cardiac tissue in the absence of the magnetic field; and
wherein the magnetic field counteracts the efflux.

105. The method of claim 99, wherein the population of stem cells are cardiac stem cells.

106. The method of claim 105, wherein the population of cardiac stem cells are cardiosphere-derived cells.

107. The method of claim 99, wherein the first antibody portion is bound to the cell surface molecule selected from the group consisting of c-kit, CD-105, CD-90, and CD-31.

108. The method of claim 107, wherein the first antibody portion is bound to the cell surface molecule CD-105.

109. A composition comprising a population of magnetically labeled stem cells suitable for treating a patient with damaged cardiac tissue comprising:
a population of stem cells linked to magnetic particles,
wherein the magnetic particles are linked to a first antibody portion and a second antibody portion,
wherein the first antibody portion is bound to a cell surface molecule on the stem cell and the second antibody portion is capable of binding to a cardiac tissue marker expressed in damaged cardiac tissue;
wherein the population of magnetically labeled stem cells are capable of retention and engraftment in a region of damaged cardiac tissue which results in improved cardiac function, thereby repairing said damaged cardiac tissue.

110. The composition of claim 109, wherein the first antibody portion and the second antibody portion are contained in a single bi-functional antibody.

111. The composition of claim 109, wherein the first antibody portion is contained on a first antibody and the second antibody portion is contained on a second antibody.

112. The composition of claim 109, wherein the population of stem cells are cardiac stem cells.

113. The composition of claim 112, wherein the cardiac stem cells are cardiosphere-derived cells.

114. The composition of claim 109, wherein the first antibody is bound to the cell surface molecule selected from the group consisting of c-kit, CD-105, CD-90, and CD-31.

115. The composition of claim 114, wherein the first antibody is bound to the cell surface molecule CD-105.


SEQUENCE LISTING (5 sequences)

SEQ ID NO: 1 - DNA, 21 bp, Rattus norvegicus
  misc_feature (1)..(21): Forward primer SRY gene
agaggcacaagttggctcaac

SEQ ID NO: 2 - DNA, 22 bp, Rattus norvegicus
  misc_feature (1)..(22): Reverse primer SRY gene
tcccactgatatcccagctgct

SEQ ID NO: 3 - DNA, 28 bp, Rattus norvegicus
  misc_feature (1)..(28): FAM-TAMRA SRY gene Probe
caacagaatcccagcatgcagaattcag

SEQ ID NO: 4 - DNA, 20 bp, Rattus norvegicus
  misc_feature (1)..(20): Forward Primer 2 SRY Gene
ggagagaggcacaagttggc

SEQ ID NO: 5 - DNA, 19 bp, Rattus norvegicus
  misc_feature (1)..(19): Reverse Primer 2 SRY Gene
tcccagctgcttgctgatc